(19) United States
(12) Patent Application Publication
(10) Pub. No.: US 2025/0078966 A1
(43) Pub. Date: Mar. 6, 2025

(54) SPECIMEN MANAGEMENT FOR CLINICAL RESEARCH STUDY SYSTEMS

(71) Applicant: Nurocor, Inc., Austin, TX (US)

(72) Inventors: Brent Allen Carlson, Minneapolis, MN (US); Kiersten Leigh Clingersmith, Boulder, CO (US); Timothy James Graser, Spring Valley, MN (US); Jolene Marie Hill, Whitefish, MT (US)

(21) Appl. No.: 18/819,925

(22) Filed: Aug. 29, 2024

Related U.S. Application Data
(60) Provisional application No. 63/579,541, filed on Aug. 30, 2023.

(51) Int. Cl. G16H 10/20

(52) U.S. Cl. CPC G16H 10/20

(57) ABSTRACT

A computing device comprising a memory and processing circuitry may be configured to implement various aspects of the specimen management techniques. The memory may store a schedule of activities that defines one or more activities, where at least one of the one or more activities includes an assay requiring collection of a specimen from at least one subject group of subjects. The processing circuitry may present a specimen management user interface with which a study designer interacts to manage the collection of the specimen for the assay, where managing the collection of the specimen includes defining default operational details for the collection of the specimen. The processing circuitry may obtain a proto-plan defining a sequence of events and a corresponding timepoints during which the collection of the specimen occurs. The processing circuitry may present, via the specimen management user interface, the proto-plan for modification.

This application claims the benefit of U.S. Provisional Application No. 63/579,541, entitled “SPECIMEN MANAGEMENT FOR CLINICAL RESEARCH STUDY SYSTEMS,” and filed Aug. 30, 2023, the entire content of which is hereby incorporated by reference.

### TECHNICAL FIELD

This disclosure generally relates to systems for managing clinical studies.

### BACKGROUND

Data standards are often developed and used as a uniform communicative structure for the transfer of data from one entity to another entity. These data structures may be used by a receiving entity so that data can be processed in a known format for efficiency and validation during processing. Regulatory agencies are becoming advocates of data standards for submission, analysis, and approval of consumable products. However, implementation of data standards is often burdensome and expensive for industry. Moreover, as a data standard is changed or updated, the implementation of the standard may need to be changed or updated, which may require significant programming efforts and further expense.

One aspect of data standards involves informal requirements for specimen management used for validating clinical research studies (which may be referred to as “clinical studies”). Specimen management is usually performed ad hoc by subject matter experts (SMEs) that specialize in tailoring assays (which is another way to refer to “clinical tests”) used to validate hypotheses undergirding the clinical study and to ensure eligibility and safety requirements are met for subjects participating in the study. These assays may include collecting specimens from cohorts of subjects participating in the clinical study, where such specimens may involve physical specimens (e.g., bodily fluid samples—such as blood, plasma, serum, urine, etc, tissue samples, and the like) and digital specimens (such as images—such as X-rays, computerized tomography—CT—scans, magnetic resonance imaging—MRI—scans, data collection for various bodily metrics—such as weight, blood pressure, pulse rate, height, etc., and the like).

Often development of specimen management specifications for a clinical study cannot occur until the clinical study has been at least partially defined, potentially resulting in delay in beginning the clinical study until various trail arms have been fully defined and agreed upon by various entities (e.g., vendors of the clinical study supporting specimen processing, regulators, clinical study managers, etc.). Further, the informal nature of specimen management standards and specification development for coordinating specimen collection may result in excessive subject testing given the ad hoc nature of specimen management in which operational details of specimen management are not fully understood given that the operational details may be stored in various files, spreadsheets, ad hoc computer programs, etc. This potential inability to fully understand the operational details of collecting specimens in support of assays may result in excessive specimen collection that may potentially be burdensome for the subjects of the clinical study. In addition, the wide diversity of operational details formats may lead to errors in specimen management specification, introducing further delay before clinical study initiation and increased expenses in clinical study preparation (e.g., specimen collection kit preparation) and execution.

### SUMMARY

In general, the disclosure describes techniques for specimen management in a digitized framework for clinical research study systems. In some examples, a system as described herein defines a data model that specifies a data standard representative of requirements for regulatory-compliant exchange of data between entities and stores metadata for the data model in a metadata repository (MDR). As described herein, the data model includes one or more client business objects representing aspects of a well-structured pharmaceutical clinical research study. As described herein, a clinical research study (also referred to herein as a “clinical study” and/or a “study”) is, for example, a clinical research project whose objectives are to test or confirm hypotheses concerning the utility, impact, pharmacological, physiological, and/or psychological effects of a particular treatment, procedure, drug, device, biologic, food product, cosmetic, care plan, or subject characteristic. Each study as a whole specifies one or more study events, (referred to herein as “study events” or “events”) and each study event of the one or more study events specifying one or more study activities (also referred to herein as “study activities” or “activities”) to be planned for the study event.

In addition to the events and activities mentioned above, the data model further includes additional study data elements (referred to herein as “study elements”, “protocol elements” or “elements”) each of which is represented as a dynamically configured property within the MDR. These properties are collected into one or more dynamically configured property groups, also specified within the MDR, with each property group representing a section of the study (referred to herein as “study section” or “section’) whose elements are cohesively related. Section examples include Planned Intervention, Inclusion Criteria and Study Model Elements among others. These properties may be aggregated into standardized data sets for purposes of regulatory submission, or used to inform other aspects of study design (e.g., the authoring of a study protocol document that specifies the study in narrative form, submission of which is also a regulatory requirement).

The system uses the metadata stored in the MDR to generate a study model for planning a clinical research study. A user interface obtains input from a user to parameterize the study template and store the parameterized template in a database. The system further generates, based on the template, a sequence of one or more study events specifying one or more study activities to be executed for the study event. In some examples, the system may further generate, based on the template, one or more study sections each of which is composed of one or more study elements. In some examples, the system may further generate, based on the template, at least one study objective and at least one study estimand or endpoint (referred to herein as “endpoint”) that may be used to facilitate generation of key activities and events within a schedule of study activities.

In some instances, objectives may specify precise endpoint descriptions, defined by ways to estimate (which may be referred to as an estimator) and a numerical result (which may be referred to as an estimate), where, for example, in a diabetes study an endpoint may be described by an estimator HbA1c (glycated hemoglobin) as a measure for blood glucose levels, and an estimate that compares the change from study baseline of HbA1c at a prespecified study timepoint. An estimand may refer to a precise description of the treatment effect reflecting a clinical question posed by a trial objective and may have different components depending on a phase and purpose of a given trial. Estimands may be mandatory for confirmatory studies.

An estimand description may not be required for exploratory objectives or for core phase 1 studies. Estimand components include a study population, a statistical variable to be measured (which again may be referred to as an endpoint), details of how to account for intercurrent events (e.g. study discontinuation), and a population-level summary of a variable.

Moreover, within the context of a clinical trial and clinical operations processes, activities can be accelerated through what is described herein as the “lean protocol process,” which is based on protocol development gates from Clinical Development Plan (CDP) through to final protocol. This “gated” process begins with the CDP/Protocol workflow as the first gate when critical data is first available and enables parallel development of the protocol, SAP and study build to begin. The early availability of critical study data elements further triggers early and parallel development of functional plans and process activities. Using feasibility as an example, study population, planned procedures and reference drugs may be identified at gate 1 and trigger development of the feasibility and strategy plan. The protocol is further refined through subsequent gates, enabling early execution of protocol feasibility to begin after gate 2 and site feasibility activities at gate 3 or protocol level 2. The same methodology applies to other functional plan development such as data management (DM) and monitoring. Most importantly, this lean process provides the shared alignment and transparency to generate and address recommendations for changes to the study design before final protocol.

The Lean Protocol process digitizes and standardizes data that may be important to the reliability of the study findings, specifically data that supports primary and key secondary estimands/endpoints. By prioritizing the critical domains and activities based on what is unique to the study such as new/unique tools/procedures or safety considerations as a result of comparator drugs or the indication, the techniques herein may drive informed decisions and risk assessment. Leveraging the early availability of the digitized study-critical elements also facilitates study build processes such as Electronic Data Capture (EDC) configuration and Randomization and Trial Supply Management (RTSM) specification.

Critical data for reporting and analysis are also identified early on in the planning phase, driving a potential early start on development of dashboards and reporting specifications. Therefore, the lean protocol process driven by data standards and the digital protocol may accelerate the lifecycle through to submission data sets that are consistent with the protocol. This “intelligent automation” through the lean protocol process may drive quality, consistency, and behaviors that focus on critical data versus the completeness and accuracy of every data point at the expense of critical aspects. The end result provided by the lean protocol process may enable a significant reduction of the clinical life cycle and submission timeline driven by a systematic quality by design process.

In addition, various aspects of the techniques may enable a graphical user interface (GUI) clinical study protocol designer (also referred to herein as “Graphical Study Designer”) that allows a study design practitioner (or, in other words, a user) to graphically model the conceptual structure of a Study Protocol in an intuitive manner and as a result of that modeling, automatically generate the Arms necessary to execute that Study Protocol. The extended functionality provided by the graphical study designer improves user efficiency and accuracy in the early stages of Study Design. Each Arm represents a path through which a Study Subject can proceed within the context of the Study.

The Graphical Study Designer application and supporting microservice may eliminate the potential for an inaccurate representation of these Arms and the resulting delays in Study Protocol finalization that would ensue from such an error. In this manner, the Graphical Study Designer provides a more capable alternative means of modeling Study Protocol Arms than a tabular capability otherwise disclosed as part of the Study Protocol Elements application provided by the system.

In addition, rather than delay specimen management, various aspects of the techniques described in this disclosure may enable subject matter experts (SME) to begin specimen management specification while activities are being specified for the study's Schedule of Activities (SOA). These activities may include assays associated with study events (which may be referred to as “events”) at different timepoints (e.g., baseline, week four (4), and week eight (8) of the clinical study) for the collection of specimens. The SME may interface with a user interface of a specimen management microservice executed by the clinical research study system to define default operational details of specimen collection and assay processing concurrent to the design of the clinical study (and prior to finalizing the design of the clinical study). Once the design of the clinical study (e.g., via the Graphical Study Designer and Schedule of Activities applications) is finalized (or in other words “locked”), the SME may again interface with the user interface of the specimen management microservice to generate default collection and aliquoting plans in support of the assays to be executed for the study's events. In addition, the SME may tailor each assay across events via override operational details that override, for the particular plan, the default operational details.

Furthermore, the specimen management microservice may attempt to create compact specimen collection plans, merging collection of the same specimens (e.g., blood) for the same cohort (which may refer to a categorized subset of the subject participating in the clinical study) to possibly reduce specimen collection and thereby reduce the burden on the subjects of the cohorts (e.g., in terms of reducing the number of collections and thereby reducing the number of distinct collection events, or the amount of the specimen collected, etc.). The specimen management microservice may allow the SME, via the specimen management user interface to override the merger of specimens and thereby split specimens when the SME disagrees with the merger (e.g., when the collections for a specific event require specialized handling in comparison to other events with similar collections).

In this way, the techniques of the disclosure may organize specimen management within the digital framework provided by the clinical research study system in a manner that allows concurrent specimen management specification during study design, while still potentially providing flexibility to allow the SME to tailor (via the above noted override, split, and merge actions) specimen collection for particular events. Through organized specimen management, the specimen management microservice may promote compact specimen collection to allay subject burden (e.g., in terms of time spent undergoing specimen collection, amounts of specimen collection) while still enabling SMEs to accommodate incomplete specimen management standards.

In one example, various aspects of the techniques are directed to a method comprising: obtaining, by processing circuitry, a schedule of activities that defines one or more activities for completing a trial arm of a clinical study, wherein at least one of the one or more activities includes an assay requiring collection of a specimen from at least one subject group of subjects in the clinical study; presenting, by the processing circuitry, a specimen management user interface with which a study designer for the clinical study interacts to manage the collection of the specimen for the assay, wherein managing the collection of the specimen includes defining default operational details for the collection of the specimen; obtaining, by the processing circuitry, a proto-plan defining a sequence of one or more events and a corresponding one or more timepoints during which the collection of the specimen occurs for the at least one subject group in the clinical study; and presenting, by the processing circuitry and via the specimen management user interface, the proto-plan for modification, including defining override operational details that override the default operation details for the collection of the specimen.

In another example, various aspects of the techniques are directed to a computing device comprising: a memory configured to store a schedule of activities that defines one or more activities for completing a trial arm of a clinical study, wherein at least one of the one or more activities includes an assay requiring collection of a specimen from at least one subject group of subjects in the clinical study; and processing circuitry configured to: present a specimen management user interface with which a study designer for the clinical study interacts to manage the collection of the specimen for the assay, wherein managing the collection of the specimen includes defining default operational details for the collection of the specimen; obtain a proto-plan defining a sequence of one or more events and a corresponding one or more timepoints during which the collection of the specimen occurs for the subject group of the subjects in the clinical study; and present, by the processing circuitry and via the specimen management user interface, the proto-plan for modification, including defining override operational details that override the default operation details for the collection of the specimen.

In another example, various aspects of the techniques are directed to a non-transitory computer-readable storage medium having instructions stored thereon that, when executed, cause one or more processors to: obtain a schedule of activities that defines one or more activities for completing a trial arm of a clinical study, wherein at least one of the one or more activities includes an assay requiring collection of a specimen from at least one subject group of subjects in the clinical study; present a specimen management user interface with which a study designer for the clinical study interacts to manage the collection of the specimen for the assay, wherein managing the collection of the specimen includes defining default operational details for the collection of the specimen; obtain a proto-plan defining a sequence of one or more events and a corresponding one or more timepoints during which the collection of the specimen occurs for the subject group of the subjects in the clinical study; and present, by the processing circuitry and via the specimen management user interface, the proto-plan for modification, including defining override operational details that override the default operation details for the collection of the specimen.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a block diagram illustrating an example system for specimen management in a digitized framework for clinical development in accordance with the techniques of the disclosure.

FIGS. 2-23 illustrate example user interfaces presented by the specimen management user interface shown in the example of FIG. 1 in performing specimen management in accordance with various aspects of the techniques described in this disclosure.

FIGS. 24-57 illustrate another example user interfaces presented by the clinical research study system shown in the example of FIG. 1 in performing specimen management in accordance with various aspects of the techniques described in this disclosure.

FIG. 58 is a flowchart illustrating example operation of the system of FIG. 1 in performing specimen management in accordance with various aspects of the techniques described in this disclosure.

Like reference characters refer to like elements throughout the figures and description.

### DETAILED DESCRIPTION

In some examples, a system as described herein defines a data model that specifies a data standard representative of requirements for regulatory-compliant exchange of data between entities and stores metadata for the data model in a metadata repository (MDR). As described herein, the data model includes one or more client business objects representing aspects of a well-structured pharmaceutical clinical research study. As described herein, a clinical research study (also referred to herein as a “study”) is, for example, a clinical research project whose objectives are to test or confirm hypotheses concerning the utility, impact, pharmacological, physiological, and/or psychological effects of a particular treatment, procedure, drug, device, biologic, food product, cosmetic, care plan, or subject characteristic. Each study as a whole specifies one or more study events, (referred to herein as “study events” or “events”) and each study event of the one or more study events specifying one or more study activities (also referred to herein as “study activities” or “activities”) to be planned for the study event.

In addition to the events and activities mentioned above, the data model further includes additional study data elements (referred to herein as “study elements”, “protocol elements” or “elements”) each of which is represented as a dynamically configured property within the MDR. These properties are collected into one or more dynamically configured property groups, also specified within the MDR, with each property group representing a section of the study (referred to herein as “study section” or “section’) whose elements are cohesively related. Section examples include Planned Intervention, Inclusion Criteria and Study Model Elements among others. These properties may be aggregated into standardized data sets for purposes of regulatory submission, or used to inform other aspects of study design (e.g., the authoring of a study protocol document that specifies the study in narrative form, submission of which is also a regulatory requirement).

The system uses the metadata stored in the MDR to generate a study template for planning a clinical research study. A user interface obtains input from a user to parameterize the study template and store the parameterized template in a database. The system further generates, based on the template, a sequence of one or more study events specifying one or more study activities to be executed for the study event. In some examples, the system may further generate, based on the template, one or more study sections each of which is composed of one or more study elements. In some examples, the system may further generate, based on the template, at least one study objective and at least one study estimand or endpoint (referred to herein as “endpoint”) that may be used to facilitate generation of key activities and events within a schedule of study activities.

In some instances, objectives may specify precise endpoint descriptions, defined by ways to estimate (which may be referred to as an estimator) and a numerical result (which may be referred to as an estimate), where, for example, in a diabetes study an endpoint may be described by an estimator HbA1c (glycated hemoglobin) as a measure for blood glucose levels, and an estimate that compares the change from study baseline of HbA1c at a prespecified study timepoint. An estimand may refer to a precise description of the treatment effect reflecting a clinical question posed by a trial objective and may have different components depending on a phase and purpose of a given trial. Estimands may be mandatory for confirmatory studies.

An estimand description may not be required for exploratory objectives or for core phase 1 studies. Estimand components include a study population, a statistical variable to be measured (which again may be referred to as an endpoint), details of how to account for intercurrent events (e.g. study discontinuation), and a population-level summary of a variable.

In addition, various aspects of the techniques may enable a graphical user interface (GUI) clinical study protocol designer (also referred to herein as “Graphical Study Designer”) that allows a study design practitioner (or, in other words, a user) to graphically model the conceptual structure of a Study Protocol in an intuitive manner and as a result of that modeling, automatically generate the Arms necessary to execute that Study Protocol. The extended functionality provided by the graphical study designer improves user efficiency and accuracy in the early stages of Study Design. Each Arm represents a path through which a Study Subject can proceed within the context of the Study.

The Graphical Study Designer application and supporting microservice may eliminate the potential for an inaccurate representation of these Arms and the resulting delays in Study Protocol finalization that would ensue from such an error. In this manner, the Graphical Study Designer provides a more capable alternative means of modeling Study Protocol Arms than a tabular capability otherwise disclosed as part of the Study Protocol Elements application provided by the system.

In addition, rather than delay specimen management specification for the study, various aspects of the techniques described in this disclosure may enable subject matter experts (SME) to begin specimen management specification while activities are being defined. These activities may include assays associated with study events (which may be referred to as “events”) at different timepoints (e.g., baseline, week four (4), and week eight (8) of the clinical study) for the collection of specimens. The SME may interface with a user interface of a specimen management microservice executed by the clinical research study system to define default operational details of specimen collection concurrent to the design of the clinical study (and prior to finalizing the design of the clinical study). Once the design of the clinical study (e.g., via the Graphical Study Designer and Schedule of Activities applications) is finalized (or in other words “locked”), the SME may again interface with the user interface of the specimen management microservice to generate default collection and aliquoting plans in support of the assays to be executed for the study's events. In addition, the SME may tailor each assay across events via override operational details that override, for the particular event, the default operational details.

Furthermore, the specimen management microservice may attempt to create compact specimen collection plans, merging collection of the same specimens (e.g., blood) for the same cohort (which may refer to a categorized subset of the subject participating in the clinical study) to possibly reduce specimen collection and thereby reduce the burden on the subjects of the cohorts (e.g., in terms of reducing the number of collections and thereby reducing the number of distinct collection events, or the amount of the specimen collected, etc.). The specimen management microservice may allow the SME, via the specimen management user interface to override the merger of events and thereby split events when the SME disagrees with the merger (e.g., when the collections for a specific event require specialized handling in comparison to other events with similar collections).

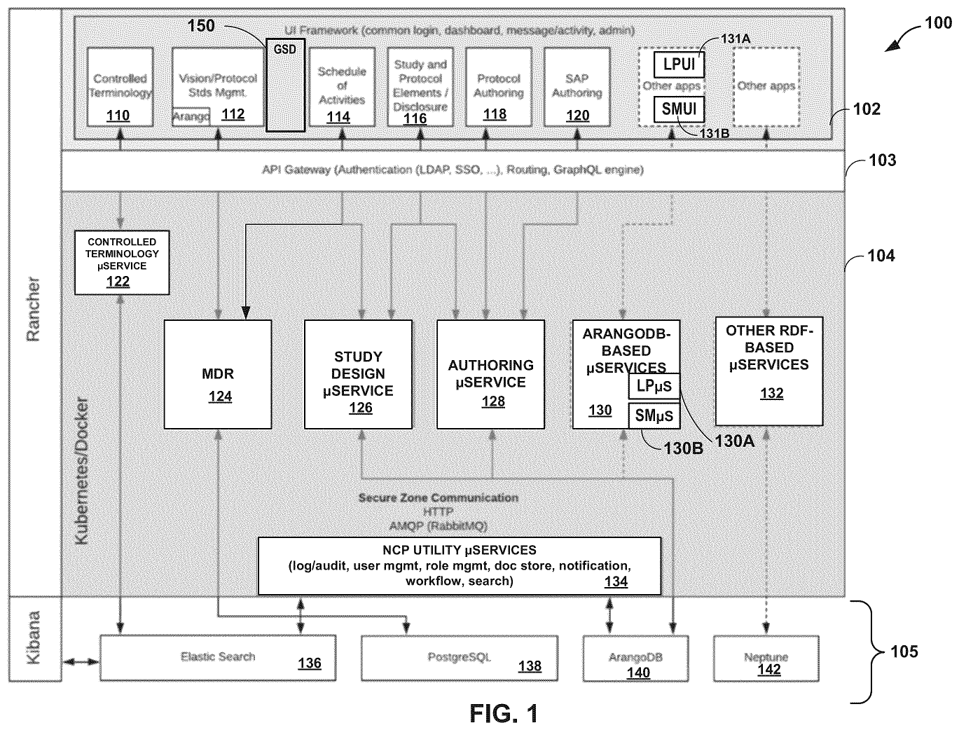

In this way, the techniques of the disclosure may organize specimen management within the digital framework provided by the clinical research study system in a manner that allows concurrent specimen management specification during study design, while still potentially providing flexibility to allow the SME to tailor (via the above noted override, split, and merge) specimen collection for particular events. Through organized specimen management, the specimen management microservice may promote compact specimen collection to allay subject burden (e.g., in terms of time spent undergoing specimen collection, amounts of specimen collection, visits/event frequency, etc.) while still enabling SMEs to accommodate incomplete specimen management standards. FIG. 1 is a block diagram illustrating example system 100 for providing specimen management in a digitized framework for clinical development in accordance with the techniques of the disclosure. System 100 includes UI framework 102, microservice platform 104, and database 105. System 100 further includes API gateway 103 which interfaces between UI framework 102 and microservice platform 104.

System 100 provides a clinical lifecycle management application suite and provides a technology platform that supports multiple means of integration and collaboration. System 100 may be architected with three core principles in mind: modularity and flexibility, scalability resiliency and security, and openness and pluggability.

System 100 may use a microservice-based architecture, which potentially provides a flexible way of building out incremental functionality, with each microservice deployed within the platform controlling its respective data and collaborating with other microservices and UI based applications via one or more APIs, with API gateway 103 serving as both a security layer and an internal routing mechanism for such APIs. Microservices also enable multiple programming languages to be used within the same deployment platform. In some examples, system 100 may use a mix of Java and node.js languages to develop fit-for-purpose components.

UI framework 102 provides a framework for various applications to provide information to a user. Typically, an application of UI framework 102 operates in conjunction with a corresponding microservice of microservice platform 104. In the example of FIG. 1, UI framework 102 includes controlled terminology application 110, which operates with controlled terminology microservice 122 of microservice platform 104 to support the ingestion of quarterly updates of controlled terminology from CDISC and NC. UI framework 102 further includes protocol standards management application (also known as Vision) 112, which operates with MDR 124 to provide clinical standards management. Schedule of study activities application 114, Study and Protocol Elements/disclosure application 116, protocol authoring application 118, and statistical analysis plan (SAP) authoring application 120 operate in conjunction with study design microservice 126 and authoring microservice 128 to facilitate the digitization and automation of clinical research studies.

Each microservice 122, 124, 126, 128, 130, and 132 communicates within the platform and to UI framework 102 through APIs. System 100 is an API-first platform, and uses a combination of GraphQL based APIs as well as REST-based APIs to support intercommunication both within the server side of the platform as well as communication to the outside world through UI-based applications and via direct API calls.

With respect to UI-based applications 110, 112, 114, 116, 118, 120, system 100 implements a common UI framework 102 that all applications consume. UI framework 102 provides a seamless cross-application navigation interface such that a user of system 100 is not aware that the user is using multiple applications. Instead, the user simply navigates from capability to capability while performing daily work. System 100 may also support multiple data persistence platforms and enables the easy addition of support for additional data platforms in the future.

In some examples, from a scalability, resiliency and security standpoint, system 100 uses a combination of Docker and Kubernetes as a deployment backbone. Docker enables the packaging up of stateless applications or components, such as microservices and UI-based applications, in a reusable fashion. Kubernetes then combines its capabilities with Docker to manage Docker instances within a runtime environment, automatically scaling those capabilities, ensuring that the instances stay up and running as well as managing restarts when needed, and otherwise providing effective administrative capability over the run-time of the platform.

An administrator may manage system 100 through Rancher, which is a popular administrative user interface plugged into the Kubernetes environment. System 100 also provides access to elastic search 136 from a log and audit standpoint through a Kibana UI front end, which is part of the ELK (Elasticsearch, LogStash, Kibana) suite. Although described with respect to a particular front end user interface (UI), e.g., Kibana UI, for accessing ELK databases, and a particular database, system 100 may utilize different frontend UIs and databases.

System 100 provides a secure and flexible perimeter around back-end microservices that are managing data via a purpose-built API Gateway 103. System 100 uses industry-hardened components within this implementation to provide core endpoint security, to give the flexibility to plug in multiple authentication approaches such as (Lightweight Directory Access Protocol (LDAP) and various types of Single Sign-On (SSO) and to support federation of GraphQL APIs. API Gateway 103 also provides other baseline capabilities, such as health check support, for the various components in the platform and a way for components to register as the components come online.

System 100 is architected to be open and pluggable via the use of APIs that facilitate loosely coupled integration. These APIs support both technical integrations around the utility capabilities of the platform as well as functional integrations that support the clinical lifecycle. Above and beyond this loosely coupled API support, system 100 supports deep integration through a well-defined set of internal microservice APIs 122, 124, 126, 130, 132 and a reusable UI Framework 102. These interfaces enable partners and customers to develop natively-deployed microservices and UI apps (which may be referred to collectively as “extensions”) that sit seamlessly alongside core capabilities that system 100 delivers.

In some examples, the runtime of system 100 is based on Docker. Docker is a container virtualization environment that enables components based on Java, node.js and other languages (e.g., programming languages, descriptor languages, etc.) to efficiently share a set of pooled runtime instances. These images are then configured to be deployed within cloud platforms or into properly-configured on-premises (so-called “on-prem”) environments in a reusable and scalable manner. Kubernetes supports platform scalability and resiliency by providing a management backbone for these Docker instances. When configuring Kubernetes, various clusters of runtime containers are established and affinity for various components being deploying into those containers are established. Kubernetes then manages those capabilities to ensure that the platform remains operational (or, in other words, stays up) and performs well. Kubernetes also provides a communication backbone in the form of a private IP network.

The architecture of system 100 builds upon common data stores by provisioning data stores of various types. In some examples, system 100 leverages relational data stores in the form of PostgreSQL 138, document type NoSQL data stores in the form of Elasticsearch 126, and combination document/graph type data stores in the form of ArangoDB 140. System 100 may also integrate with other data stores, such as RDF triple stores. Rancher provides a UI for managing Kubernetes instances, and Kibana provides effective log and audit retrieval and analysis capabilities on top of Elasticsearch 126.

System 100 includes a security layer between the server side of the platform and the outside world, e.g., the Internet or other public networks. System 100 creates a secure “green zone” within Kubernetes taking advantage of the private IP network provided by Kubernetes. System 100 also supports internal message queue-based communication through Advanced Message Queueing Protocol (AMQP) provided by a RabbitMQ instance. All of this is secured behind a purpose-built application programming interface (API) Gateway 103. API Gateway 103 provides a secure perimeter for API exposure to the outside world, both to applications of system 100 as well as for direct integration from other partner platforms or customer direct usage. API Gateway 103 also enables pluggable authentication through various authentication strategies, including, for example, lightweight directory access protocol (LDAP), header based single sign-on (SSO), security assertion markup language (SAML) based SSO, or open identification (OpenID). Other authentication strategies may also be enabled via this pluggable authentication framework. API Gateway 103 integrates a GraphQL engine that allows microservices 122, 124, 126, 128, 130, 132 to provision their portion of the data graph into a federated GraphQL API. This GraphQL API aggregates data content across all the various microservices supporting system capabilities. This provides increased client-side ease of use and efficient access to content provided by system 100.

System 100 uses both a common UI framework 102 and a back-end set of utility microservices 134. UI framework 102 provides all applications with common login, common user session management and seamless cross-application navigation, with applications registering into the framework such that they are able to communicate and navigate with each other. UI framework 102 also provides various shared end user and administrative UI elements including a library of common widgets and other components that are available for application development. In the back-end, system 100 provides a suite of utility microservices that in essence provide the shared technical capabilities needed in order to build out an enterprise-class application platform suite. Capabilities such as dynamic properties, comments, change requests, logging, audit, user management, role and permissions management (i.e., access control), document store, reporting, notifications through multiple channels, integrated workflow and integrated cross-application search are part of utility microservice suite 134.

System 100 enables data standards management by an underlying metadata repository (MDR) 124 that is embedded within system 100. MDR 124 is an ISO 11179-based MDR that supports the provisioning of configurable content. In some examples, MDR 124 is a regulatory database based on a governance process that defines a protocol model for storing metadata. This metadata is managed to ensure that version control is maintained as the platform and its capabilities mature over time. Robust workflow and governance capabilities of the system 100 apply both as new clinical data standards (CDISC and others) are specified and provisioned and as new program, protocol, and study standards are defined and refined.

In some examples, MDR 124 is prepopulated with clinical data acquisition standards harmonization (CDASH) to study data tabulation model (SD™) transformation rules. As organizations continue to build out more efficient clinical lifecycle execution, MDR 124 may incorporate additional rule-based transformations (including internal standards transformations and transformations from SD™ to analysis data model—ADaM). MDR 124 may express rules in narrative or executable form (for example statistical analysis system—SAS—program fragments). Additional models and transformation rules between models may be incorporated as required to support clinical trial modeling, definition, and execution.

MDR 124 is an ISO 11179-based MDR that support the provisioning of configurable content. This metadata is managed to ensure that version control is maintained as the platform and its capabilities mature over time. Robust change request, workflow, and governance capabilities of the system 100 apply both as new clinical data standards (CDISC and others) are specified and populated, and as new program, protocol, and study standards are defined and refined.

MDR 124 is exposed to data standards curators and other standards management personnel through Protocol Standards Management application 112 (also known as Vision). Vision provides a very flexible yet end user centric view over MDR content. MDR 124, by its very nature, includes technical components and Vision allows the presentation of MDR content in a context that is meaningful to managers of that content. Controlled terminology microservice 122 and application 110 suite augment MDR 124 by supporting the ingestion of quarterly updates of controlled terminology from CDISC and NCI. Thus, MDR 124 may provide a flexible application that supports effective identification of the intersection between new terminology and existing internal terminology defined by clinical organizations and helps customer organizations manage the impact of adopting newly defined terminology on a case-by-case basis. API Gateway 103 in turn exposes data standards APIs that allow system 100 to support downstream data platform provisioning with other customer or vendor platforms.

In addition to supporting direct content access externally via GraphQL API calls, the platform also provides reporting microservice 134 which exposes platform content via configured REST APIs. Each configured API is exposed via API Gateway 103 and is associated with an internal GraphQL query that retrieves content of interest for the report. Query results are then provided to a configurable formatting engine exposed by microservice 134. The formatting engine collects, organizes, and transforms the contents of query results into the desired output in the form of java script object notation (JSON), extensible markup language (XML), Microsoft Excel spreadsheet (XLSX), comma separated values (CSV) and/or portable document format (PDF) as required by the expected user of the configured API. In one example, reporting microservice 134 maps an inbound REST API request to a specific service configuration that defines how to fetch the data needed for the response, how to traverse that data, and how to prepare the response. The incoming request mapping is routed based on the parameterized resource path of the incoming resource request.

Once mapped, the service configuration informs which GraphQL query to run against the system's federated GraphQL interface. This query may be tuned to specify the exact data attributes needed to satisfy the request. The GraphQL response is a JSON object that is further processed by reporting microservice 134. The JSON object is traversed based on the service configuration and is defined in terms of data views, each of which consists of data selectors and (further recursive) data views. During traversal, the recursion algorithm sends events to a formatter when a view starts, a data selector delivers data, or when a view ends. Formatters are agnostic to the actual request, data source or traversal. They prepare a data export based on the events such formatters receive. Each formatter is specialized for a specific export format based on the media type of the original request. Reporting microservice 134 may support response formats for Excel, CSV, JSON, XML, and PDF media types. In another example, the reporting microservice configuration supports the application of XSLT transformations for XML based media types.

Study Design microservice 126 sits at the core of the clinical lifecycle management suite provided by system 100. Study Design microservice 126 provides a fully digital clinical research study representation ranging from study initialization parameters such as Therapeutic Area and Indication, study-specific objectives and endpoints, all the way through to a full fidelity schedule of study activities with study events (which may also be referred to as “events”) and study activity (which may also be referred to as an “activity”) items that are to be executed for those study events. Both industry-wide and corporate clinical standards may be automatically sourced from MDR 124 in support of Study Design microservice 126.

Study and Protocol Elements application 116 manages the wide range of elements organized into pages and sections and that are necessary to populate and define a fully specified study. One aspect of this application is the Objective and Endpoint editor. This editor exposes predefined templates managed within the MDR 124, providing a study designer with an intuitive and easy to use UI when defining primary, secondary, or exploratory Objectives and Endpoints for the study under development. These Objectives and Endpoints establish a basis for the scientific hypotheses to be experimentally assessed by the Study. Study and Protocol elements populated via this application are persisted in study design microservice 126, where the elements become available for downstream systems use including automated disclosure capabilities to relevant authorities.

Schedule of study activities application 114 provides a fully digitized clinical research study schedule development environment that leverages objectives and endpoints that have been previously specified to provide automated suggestions for schedule study events as well as study activities to be executed during those study events. Schedule of study activities application 114 directly consumes industry and corporate data standards automatically from MDR 124 such that once the user has completed schedule definition, a full fidelity schedule is then stored in study design microservice 126, synchronized back to MDR 124 for impact analysis purposes and ultimately enabled for export (via file extract, API invocation, etc.) to downstream customer or vendor platforms. In other examples, the full fidelity schedule may be stored in one or more special purpose microservices for impact analysis and export purposes.

System 100 provides an additional protocol standards management capability in the area of data standards via MDR 124. A clinical data standards management protocol standards management application facilitates defining reusable objective and endpoint templates along with other data elements in support of clinical research study lifecycle applications.

Authoring microservice 128 and application suite 120 provide protocol authoring capabilities. In one example, this suite supports a fully digitized implementation of a protocol model of a clinical research study along with other templatized content. Authoring microservice 128 and application suite are natively integrated with study design microservice 126 and MDR 124. Authoring microservice 128 is fully templatized so that one may apply additional templates to support customer-specific protocol formats, and support alternative documents such as statistical analysis plans.

Microservice developers may pick from a wide range of programming languages and data persistence platforms. Specifically, system 100 may incorporate both portfolio and program level applications such as Portfolio Management, Indication Analysis, Target Product Profile (TPP), Clinical Development Plan (CDP) and Protocol Synopsis as well as study execution centric applications and support of topics such as feasibility and recruitment, risk management and supply management. These additional applications are examples only and not intended to limit the scope of system 100.

System 100 uses database 105 to store, manage, and retrieve content for clinical development. Database 105 may take various forms, such as a relational database, a document-based database, or a graph-based database. In the example of FIG. 1, database 105 is implemented with Elasticsearch 136, PostgreSQL 138, ArangoDB 140 and/or Neptune (an example of an RDF Triple Store database) 142. In some examples, database 105 implements GraphQL queries and mutations to manage data. For example, study design microservice 126 implements GraphQL APIs that invoke database 105 as an efficient and flexible way to retrieve clinical development content.

Microservices 122, 124, 126, 128, 130, 132, and 134 are “GraphQL first” in nature. Microservices 122, 124, 126, 128, 130, 132, and 134 expose core Create/Read/Update/Delete (CRUD) actions on their business objects via GraphQL queries and mutations. GraphQL, originally developed by Facebook, Inc. as part of its microservice-based platform architecture, provides two possible features that make GraphOL well-suited for such development: federation and field (attribute) selectivity.

GraphQL supports the concept of API federation, where each microservice contributes its business object declarations to an all-encompassing graph spanning the entire set of business objects deployed into the platform. This federated view is exposed by a single GraphQL API endpoint. As each new microservice 122, 124, 126, 128, 130, 132, and 134 is deployed into the platform, the microservice augments this object graph with the new objects for which it has responsibility. A GraphQL API consumer can in turn initiate a single API query that spans as much of the graph as is needed, with server-side infrastructure efficiently merging the content to support the API request.

Field selectivity in GraphQL is a natural complement to business object federation, as GraphQL allows API consumers to precisely identify which object fields (attributes) are to be returned in query results. This eliminates the potential concern of very large datasets being returned to the client in response to a broad query invocation, while at the same time eliminating the need to implement multiple purpose-specific APIs supporting various combinations of object content to suit the needs of the client. Because GraphQL supports field selectivity, it is easy to modify a full query to database 105 by removing various elements to produce a result that aligns with desired view of the template graph aligned for ease of use to the customer. The result may in turn be transformed from JSON into other representations (e.g., HTML) via reporting microservice 134, UI framework 102, or other aspects of system 100.

System 100 may operate to generate dynamically configured applications composed of pages and sections within pages. Contextual properties such as ordinality, read-only, multivalued, and required are used by the UI to present the property correctly within the page section in question. Different property subtypes require specialized context properties; for instance, ordered literal types such as integer, decimal, date, and timestamp include optional properties such as lower and upper bounds and Boolean flags that indicate inclusive or exclusive bounds settings. Dynamically retrieved types may include user specified attributes, such permission name (derived from system configuration used to specify access control microservice 134 behavior) and associated resource type, and include optional properties such as whether the current user is to be included in the dynamically retrieved results for the property.

Enumerated properties may be specified by value lists within MDR 124, or those value lists may be dynamically retrieved from sources external to MDR 124 (e.g., a list of platform users with a specified role may be retrieved from the system's User microservice in concert with the Access Control microservice, a list of valid Protocol Numbers may be retrieved from an external Clinical Trial Management System (CTMS)). Compound property types (which compose two or more elemental property types) may also be specified within the system. One example of a compound property type is a representation of a Study Event composed of a period type (enumerated value list composed of values such as Day, Week, Month) and integer. The Study Event property type may support automatic generation of Study Events via Endpoint Template parameterization.

Conditional property specifications, where default contextual properties are modified given a specific set of conditions, are also supported. For example, a property A may become editable only when a property B contains a specific value, or when property B is populated with any value. Conditions may span multiple specified values for a designated property (e.g., property C must be set to one of values x, y, or z for the condition to be in effect) and may span multiple designated properties (e.g., the conditional rules for properties D, E and F must all be met for the condition to be in effect).

In general, system 100, operating in accordance with the techniques of the disclosure, enables the digitization of a framework for clinical development. For example, study design microservice 126 uses metadata stored in MDR 124 to generate a study template for performing a clinical research study. UI framework 102 obtains input from a user to parameterize the study template and store the parameterized template in database 105. User input includes an Effective Date parameter which is defaulted to the current date and may be modified by the user. The Effective Date value is used to automatically retrieve the correct version of study and protocol standards from which the study template will be generated.

As such, different versions of the study template may be associated with a corresponding Effective Date parameter and thereby facilitate retrieval of a correct version of the study template representative of the governance process identified by the Effective Date parameter. The correct version is selected by identifying the version with a specified date most recently prior to the Effective date specified for the study. For example, if three versions of the study template exist, with version 1 specified date set to Jan. 31, 2018; version 2 set to Oct. 15, 2019; and version 3 set to Dec. 31, 2020, then Study A with an effective date of Jul. 15, 2019 would be assigned version 1, Study B with an effective date of Jan. 15, 2020 would be assigned version 2, and Study C with an effective date of Apr. 30, 2021 would be assigned version 3. The ability to specify an Effective Date may be useful for long-running Clinical Development Programs (e.g., for an Oncology Therapeutic Domain) where Studies within a Program span years and for which a consistent set of standards must be applied.

Study design microservice 126 generates, based on the template and potentially combined with user input, a sequence of one or more study events specifying one or more study activities to be executed for the study event and study elements associated with the clinical research study. As described herein, a “study event” refers to a checkpoint or milestone during a clinical research study at which specific study activities are performed. The study event specifies one or more study activities to be executed for the corresponding study event. Each study activity may be, e.g., a clinical assessment or procedure to be performed on a subject (such as specimen collection), a data element to be measured from the subject, an assemblage of already existing data about the subject, or collection of non-treatment related data (e.g., healthcare utilization) from the subject.

The detailed specification of a study activity is based on data collection standards (e.g., CDASH) as governed within MDR 124, thus linking these collection standards (and by extension tabulation, analysis and other data standards via transformation rules also governed in MDR 124) to the study template and protocol standards also specified within that template. For example, a study event for a study objective may occur on a periodic basis (e.g., daily, weekly, or monthly) and specify that a study activity to be performed, for example, that a subject's blood pressure and heart rate be monitored. A clinical research study may be quantized into a sequence of such study events and study activities, and thereby objectively defined and measured.

Study elements are data elements that a regulatory authority may require the collection of during the clinical research study. Further, the regulatory authority may require the submission of such study elements to the regulatory authority as part of reporting requirements of the clinical research study. System 100 enables the collection of study element data via configuration of properties and property groups specified as part of the study template and managed within MDR 124. System 100 manages these configured elements as configured sections within study design microservice 126 and directly persisted via dynamic property microservice, one of the utility microservices 134. These configured sections also support the Lean Protocol process as disclosed herein. Dynamic property microservice 134 is configured to accept study elements by study design microservice 126 through advanced message queuing protocol (AMQP) messages informing dynamic property microservice 134 of the study element sections specified in the template used for the study. Use of asynchronous messaging in this manner may preserve loose coupling between microservices.

In some examples, the specific types of study elements are mandated by the regulatory authority (e.g., as CDISC submission data sets). Examples of study elements include data related to pharmaceutical administration, sponsor information, study planning, or study extensions. Study elements related to pharmaceutical administration may include a dose formulation of the pharmaceutical studied in the clinical research study, a route of administration, a dose per administration, a dose regimen, dose units, or dosing frequency. Study elements related to sponsor information may include a responsible party, a sponsor status, a sponsor name, sponsor contact information, a sponsor legal address, a sponsor telephone number, or a sponsor email address. Study elements related to study planning may include a planned number of subjects, a planned treatment duration, a stable disease minimum duration, a confirmed response minimum duration, a planned trial duration, or study stop rules. Study elements related to study extension may include an extension trial indicator, a substudy planned indicator, or substudy details.

In some examples, study design microservice 126 may further generate, based on the template, at least one study objective and at least one study endpoint that may be used to facilitate generation of key activities and events within a schedule of study activities. A study objective specifies a hypothesis for the clinical research study. A study endpoint specifies a means by which the hypothesis is evaluated (e.g., a lab test—which may represent one example of an assay, physiological measurement—which may represent another example of an assay, or other data point). Each study event may be linked to a study objective and an associated study endpoint of the study objective. For example, a study endpoint is defined within the scope of a study objective, and the study endpoint is the means by which one establishes connectivity to a corresponding study activity for a study event. In other words, the study endpoint allows for the definition of one or more relevant study activities to be executed in associated study events. In some examples, the study endpoint is connected to one or more study activities via introduction of a Biomedical Concept resource type within MDR 124.

By defining a hypothesis through a study objective and a means for testing the hypothesis with a corresponding study endpoint, system 100 may generate a sequence of study events and study activities to be performed for each study event such that system 100 may objectively define the clinical research study as well as a method for implementing and carrying out the clinical research study. Various applications executing within microservice platform 104 may generate a schedule of study activities and study events to implement the study, generate reporting information and documentation, and/or determine a cost estimate for each study activity of the study.

As a further example, study design microservice 126 enables the specification of repeated activities during a single study event. Such a specification is called a timepoint schedule. Consider the scenario where a study event X includes drug administration (Exposure) followed by repeated measurements of Vital Signs, e.g., 10 min, 20 min, and 30 min after drug exposure. In this scenario, the Exposure study activity is marked once on the main schedule for study event X and a timepoint schedule with three timepoints is created for study event X. The Vital Signs study activity is then scheduled for observation at each of the timepoints.

To support the digitized framework for clinical development described herein, MDR 124 extends the data model set forth in ISO 11179. For example, MDR 124 implements a data model that specifies a data standard representative of requirements for regulatory-compliant exchange of data between entities and stores metadata for the data model. As described herein, the data model includes one or more client business objects representing aspects of a well-structured pharmaceutical clinical research study. A clinical research study is, for example, a research project whose objectives are to test or confirm hypotheses concerning the utility, impact, pharmacological, physiological, and/or psychological effects of a particular treatment, procedure, drug, device, biologic, food product, cosmetic, care plan, or subject characteristic.

MDR 124 extends each client business object to represent a clinical research study or associated study element, whereas each clinical research study in turn specifies one or more study events. Each event specifies one or more study activities to be executed for the corresponding study event. MDR 124 further extends the data model of ISO 11179 to include one or more properties for each of the client business objects that organize the client business objects into one or more property groups. These properties and property groups are used to represent both cohesive collections of study elements (i.e., sections) and configurable elements of objective and endpoint templates.

The extended data model implemented by MDR 124 enables the digitization of clinical research studies as described herein by templatizing various data for clinical research study such that clinical research studies may be standardized and preformatted, and specific instances of clinical research studies may be rapidly designed. Such techniques as described herein enable the digitization of clinical research studies, clinical research study management, and clinical research study reporting requirements so as to possibly obviate aspects of the performance of clinical research study management by hand. Therefore, the techniques of the disclosure may improve efficiency and data quality, reduce submission cycle times, reduce the cumbersome administrative overhead of clinical research study management by hand, as well as reduce the potential for error or omission in critical recordkeeping.

As described herein, data for clinical research studies generated in accordance with the techniques of the disclosure are stored in database 105. The techniques disclosed herein further enable the use of a “lazy” copy algorithm to copy elements stored in database 105. Furthermore, techniques are disclosed herein for updating or deleting elements of the database where a lazy copy has been performed. In this manner, the techniques of the disclosure may allow for the use of lazy copy to increase performance of copying elements in a graph, even where the elements are members of a complex database such as a graph database, without substantially impacting the user experience or computing performance.

In addition, Schedule of Activities application 114, Study and Protocol Elements application 116, and Protocol Authoring application 118 (executed against Study Design microservice 126) may accommodate graphical user interface (GUI) study designer (“GSD”) 150 (which exists as a separate graphical user interface application that complements one or more of associated applications 114-118 as well as possibly additional apps that are not shown in the example of FIG. 1) that enables graphical design of a study protocol. When GUI study designer 150 is present, certain operations of associated applications may be rendered as read-only; in other words, these operations are supplanted by the more advanced graphical design capabilities of GUI study designer 150 thereby improving the overall ease of use of system 100 for the end user.

Further, system 100 includes Lean Protocol application 131A (which may be referred to as Lean Protocol user interface 131A—LPUI 131A) and Lean Protocol microservice 130A (“LPptS 130A”) that implement the functions of the lean protocol system for presenting a clinical study protocol as a sequence of a plurality of stages described herein. A stage specifies a logical grouping of study development tasks and may comprise multiple concurrently-executable processes (also referred to herein as “swimlanes”) with the intent that these study development tasks are enabled earlier in the study development process than would be otherwise possible via the appropriate grouping and locking of the necessary prerequisite study protocol content sufficient to enable those actions. In other words, Lean Protocol application 131A is a formalization or instantiation of the principle that Clinical Study design can be made more efficient by decomposing what is currently treated as a monolithic set of work into multiple concurrently executed “gates,” each of which frees up specialized processes/tasks that are necessary for study execution to begin.

Gates may optionally be grouped into phases, which may be logical in nature and serve to represent relative timing from inception of the clinical study design work. Gates may address specific topics (e.g., feasibility and recruitment) that in turn may be present in multiple phases with each phase representing a potential refinement of design content informed by the results of gate-enabled prior phase activity.

In one example, for a simple study design (e.g., Phase I safety trial) these gates may be fully independent of each other and grouped under a single stage. In another example, for a more complex study design (e.g., Phase III efficacy trial) gates may be assembled into multiple stage groupings, where each stage progressively refines the study protocol content from earlier stages and as a result enables the study development tasks associated with gates of those stages to also be further refined.

Lean Protocol conceptually sits above all other Study development application functionality of system 100 of FIG. 1 (including Graphical Study Designer application 150, Schedule of Activities application 114, and other apps not expressly described herein). Each such application may produce one or more “conditions” that feed into “gates;” these gates in turn free up downstream processes/tasks once approved. In one example, these conditions are based on the locking of Study The GUI clinical study protocol designer (also referred to herein as “Graphical Study Designer”) 150 allows a study design practitioner to graphically model the conceptual structure of a Study Protocol in an intuitive manner and as a result of that modeling, automatically generate the Arms necessary to execute that Study Protocol. The extended functionality provided by graphical study designer 150 improves user efficiency and accuracy in the early stages of Study Design. Each Arm represents a path through which a Study Subject (which is another way to refer to a subject of the clinical study) can proceed within the context of the Study.

For example, in a double-blinded Study where a drug candidate is evaluated against a placebo with neither the medical practitioner nor the subject being aware of which substance the subject is being given, two Arms are necessary, one for the placebo path and one for the active drug path, with a randomization rule distributing the subjects within the study between the two Arms. Study Protocols may become much more complex than this, and one of the potential challenges for a Study Designer is to accurately decompose a complex Protocol's design into the correct set of Arms that represent the full set of valid Study Protocol paths through which a subject can proceed. Graphical Study Designer application 150 and supporting microservice eliminates the potential for an inaccurate representation of these Arms and the resulting delays in Study Protocol finalization that would ensue from such an error. In this manner, Graphical Study Designer 150 provides a more capable alternative way of modeling Study Protocol Arms than the tabular capability previously disclosed as part of Study and Protocol Elements/Disclosure application 116.

In addition, Graphical Study Designer 150 produces standardized CDISC-compliant datasets representing Trial Elements (TE) and Trial Arms (TA). In combination with Study and Protocol Elements application 116 production of the CDISC-compliant datasets Trial Inclusion/Exclusion Criteria (TI) and Trial Summary Elements (TS) and the Schedule of Activities application 114 production of Trial Visits (TV), system 100 as a whole is capable of automatically producing the full complement of CDISC-compliant Study Design datasets that are required as part of a regulatory submission and are typically assembled in a manual manner from disparate information. In this way, the ability to automatically assemble these datasets may provide significant value to the end user in terms of accuracy and efficiency. System 100 of FIG. 1 provides a third means of Arm definition directly embedded within the Schedule of Activities application.

The capabilities of graphical study designer 150 may naturally flow into the previously disclosed Schedule of Activities (SOA) application 114. As part of a Study Protocol's specification, each Arm is associated with a Schedule. Within a Study Protocol, multiple Arms may share the same Schedule (as in the case for the double-blinded efficacy study described above, where every subject experiences the Study in an identical manner with the exception of the substance the subject is given), each Arm may be assigned a unique Schedule, or some combination of shared and unique Schedules may be assigned.

During the period when a study protocol is under development, Arms and Schedules may be decoupled. This allows a user of the Schedule of Activities application 114 to detach an Arm from a Schedule in case the Arm design needs to be updated, but the configuration of the Schedule needs to be preserved. In this example, after updating the Arm design the user can attach the temporarily decoupled Schedule again to one or more Arms of the updated Arm design. When a Schedule is not attached to any Arms it is considered an orphaned Schedule.

As noted above, orphaned Schedules are allowed only during protocol development. Upon a user's attempt to lock the Schedule of Activities section (where such “lock” may indicate the Schedule of Activities is finalized and no validation errors were encountered), Study Design microservice 126 will validate that no orphaned schedules are present in the study protocol and prevent the section lock from occurring if this is not the case. In other words, as a result of this Arm to Schedule linkage which occurs within the Schedule of Activities application 114, a Schedule may (temporarily) exist without being attached to any Arm (where such Schedule may be referred to as an orphaned Schedule). Support for decoupling of Arms from Schedules accommodates a potential need for designers to be able to delete one or more Arms under development without losing already created Schedules, possibly in the context of using graphical study designer 150. As a result, Schedules are assigned a human-readable and meaningful label so they can be identified by the user independent of the existence of Arms.

As a peer application to graphical study designer application 150, schedule of activities application 114 and underlying study design microservice 126 may support the following Arm and Schedule Management requirements:
1. When an Arm is newly created (regardless of source), it has no related Schedule.
2. A newly created Arm A without a Schedule can acquire a schedule in three ways:
2.1. First, it may attach an existing Schedule X to Arm A. It is possible that X was orphaned before becoming attached to Arm A.
2.2 Second, it may create a new empty Schedule X and attach it to Arm A.
2.3 Third, it may create a new Schedule X as a copy of an existing Schedule Y and attach X to Arm A.
3. Given Arm A related to a Schedule X, it is valid to relate A to another existing Schedule Y. Schedule X may become orphaned as a result of this action. It is possible that Y was orphaned before becoming attached to Arm A.
4. Detach an existing Schedule X from an Arm A. The Arm then returns to the same state as in (1), e.g., an Arm with no related Schedule. Schedule X may become orphaned as a result of this action.
5. The logical action to change an existing Schedule X of an Arm A to a copy of another existing Schedule Y is not directly supported in the Schedule of Activities application 114, as this functionality can be accomplished by first doing (4) followed by (2.3).
6. An Arm without a Schedule can be deleted (unless a Lean Protocol lock is in place). Arm deletion occurs in the application used to generate the initial set of Arms for the Study Protocol.
7. An Arm A with a related schedule X may also be deleted. This results in deletion of Arm A, but not in deletion of associated Schedule X. Schedule X may become orphaned as a result of this action.
8. A Schedule may only be deleted if it is orphaned. To delete a Schedule X that is not orphaned, the user first detaches all Arms from schedule X.

Graphical study designer 150 may operate as an independent application, or an Arm Table Editor may execute within the Study and Protocol Elements application 116. In both cases the intention is managing Arms (without thinking of Schedules): create arms, update arm attributes, delete arms. For example, creating an Arm defines a new Arm without a related Schedule as in (1) above. Updating Arm attributes is allowed, even if a Schedule is attached to the arm. Deleting an Arm (when not locked) is possible irrespective of whether a Schedule is attached following (6) and (7) above.

A user who does not use the Schedule of Activities application 114 has the capability to manage Arms without ever creating Schedules. If the user has access to Schedule of Activities application 114, creating Arms may not in itself create the overhead of automatically creating Schedules. In combination with Lean Protocol locking, the Arms have been given full consideration by the user before beginning Schedule definition. This may provide separation between managing Arms vs managing Schedules. In one example, a user may choose to enable parallel Arm and Schedule development, whereas in another example a user may choose to configure platform validation rules such that the capability of creating and editing Schedules is prevented until the Arms section has been successfully locked.

When the graphical study designer application 150 is deployed, the Arm Table within study protocol elements application 116 may be set to read-only. The user may manage Schedules dialog within schedule of activities application 114. If the schedule of activities application 114 is standalone (meaning without arm management by study and protocol elements application 116, Arm table, or graphical study designer application 150), then the dialog has two sections: Manage Arms and Manage Schedules. If schedule of activities application 114 is deployed with a separate application (either graphical study designer 150 or study and protocol elements application 116) for Arm management, then the dialog has only the second section Manage Schedules (without user actions to create or delete arms).

The aforementioned is a specific example of the general platform (which is another way of referring to system 100 or any system set forth in this disclosure) concept of deployment-sensitive application functionality. As such, each application deployed into a platform instance is aware (e.g., via registration or some other process) of all other deployed applications and, based on desired behavior, may enable or disable aspects of its functionality based on the presence or absence of other platform applications within the deployment. Another such example is the enabling or disabling of section unlock capability within the suite of study design-related applications (graphical study designer 150, study and protocol elements 116, and schedule of activities 114) based on the presence or absence of lean protocol application 131A within the deployment.

When present, the Manage Arms action Create Arm within the schedule of activities application 114 should only create the Arm, but not a default Schedule for that arm, consistent with (1) and the way Arms are created in study and protocol application 116 or graphical study designer 150. The result of the create arm action is that the Arm is added to the Manage Schedules table, but with no value for Schedule. Where system 100 provides a Manage Arms section, then an Arm (with or without a Schedule) can be deleted from the Manage Schedules section following (6) or (7) above.

Within the Manage Schedules table of the schedule of activities application 114, graphical study designer 150 may display only Arm Label from the Arm attributes. This attribute is read-only. Schedule of activities application 114 may assign a Schedule a unique label. When creating a new Schedule, the user provides a label for the Schedule. User actions for (2), (3), (4) and (8) are required for the Manage Schedules section of the dialog.

With respect to the Lean Protocol, locking does not change the foregoing, but constrains which of the actions are possible in graphical study designer 150, Arm Table Editor, and schedule of activities application 114, depending on the locking states of the apps. Without locking capabilities, all of the above may still consistently work (particularly in the case of an Schedule of Activities-only deployment). Locking constraints may force the user to do things in the correct order from a domain perspective.

One aspect of data standards involves informal definitions for specimen management used for validating clinical research studies (which may be referred to as “clinical studies”). Specimen management specification for a clinical study is usually performed ad hoc by subject matter experts (SMEs) that specialize in tailoring assays (which is another way to refer to “clinical tests”) used to validate hypotheses undergirding the clinical study. These assays may include collecting specimens from cohorts of subjects participating in the clinical study, where such specimens may involve physical specimens (e.g., bodily fluid samples—such as blood, plasma, serum, urine, etc, tissue samples, and the like) and digital specimens (such as images—such as X-rays, computerized tomography—CT—scans, magnetic resonance imaging—MRI—scans, data collection for various bodily metrics—such as weight, blood pressure, pulse rate, height, etc., and the like). Cohorts may refer to a subset of the subjects that share one or more characteristics (such as nationality, weight, weight range, age, age range, height, height range, pigmentation, race, sex, blood type, handedness, illness, symptom, disease, etc.).

Often development of specimen management specifications for a clinical study cannot occur until the clinical study has been at least partially defined, potentially resulting in delay in beginning the clinical study until various trail arms have been fully defined and agreed upon by various entities (e.g., vendors of the clinical study supporting specimen processing, regulators, clinical study managers, etc.). Further, the informal nature of specimen management standards and specification development for coordinating specimen collection may result in excessive subject testing given the ad hoc nature of specimen management in which operational details of specimen management are not fully understood given that the operational details may be stored in various files, spreadsheets, ad hoc computer programs, etc. This potential inability to fully understand the operational details of collecting specimens in support of assays may result in excessive specimen collection that may potentially be burdensome for the subjects of the clinical study. In addition, the wide diversity of operational details formats may lead to errors in specimen management specification, introducing further delay before clinical study initiation and increased expenses in clinical study preparation (e.g., specimen collection kit preparation) and execution.

In accordance with various aspects of the techniques described in this disclosure, rather than delay specimen management specification for a clinical study, system 100 may include a specimen management user interface (SMUI) 131B that enables subject matter experts (SME) to begin specimen management specification while activities are still being defined. These activities may include assays that trigger study events (which may be referred to as “events”) at different timepoints (e.g., baseline, week four (4), and week eight (8) of the clinical study) for the collection of specimens from a cohort (which is another way to refer to a categorized subset—where subset is used to denote less than all or all, but does not include a zero element subset) of subjects participating in the clinical study. The SME may interface with SMUI 131B of a specimen management microservice (SMμS) 130B executed by clinical research study system 100 to define default operational details of specimen collection concurrent to the design of the clinical study (and prior to finalizing the design—and in this example the Schedule of Activities—of the clinical study). Once the design of the clinical study (e.g., via Graphical Study Designer 150) is finalized (or in other words “locked”), the SME may again interface with SMUI 131B of specimen management microservice 130B to generate default specimen collection plans for the study, and subsequently tailor each assay across events via override operational details that override, for the particular event or events associated with a specimen management plan, the default operational details for that plan.

Furthermore, specimen management microservice 130B may attempt to create compact specimen collection plans, merging collection of the same specimens (e.g., blood) for the same cohort (which again may refer to a categorized subset of the subject participating in the clinical study) to possibly reduce specimen collection and thereby reduce the burden on the subjects of the cohorts (e.g., in terms of reducing the number of collections and thereby reducing the number of distinct collection events, or the amount of the specimen collected, etc.). Specimen management microservice 130B may allow the SME, via SMUI 131B, to override the merger of specific specimen collection activities across events into a single plan and thereby split events away from that plan when the SME disagrees with the merger (e.g., when the collections for a specific event require specialized handling in comparison to other events with similar collections)

In operation, specimen management microservice 130B may obtain a schedule of activities that defines one or more activities specified within one or more events for completing a trial arm of a clinical study, where at least one of the one or more activities includes an assay (e.g., a lab test, a clinically administered monitoring assay, etc.) requiring collection of a specimen from at least one cohort of subjects in the clinical study. Specimen management microservice 130B may interface with, as an example, the Study Design microservice 126 to obtain the Schedule of Activities, which may be unlocked (or in other words, not formally finalized) or locked (or in other words, formally finalized). Study Design microservice 126 and Specimen Management microservice 130B may operate according to an advanced message queuing protocol (AMQP) that defines an application layer protocol by which to instantiate a Biospecimen and its assays based on AMQP messages that are sent and thus arrive via a subscribe and pull mechanism.

Specimen management microservice 130B may interface with SMUI 131B to present a specimen management user interface with which a study designer (such as an SME) for the clinical study interacts to manage the collection of the specimen for the assay. The SME may interface with SMUI 131B to manage the collection of the specimen by, as one example, defining and/or overriding default operational details for the collection of the specimen. The specimen management microservice may store default biospecimen, assay and operational details information for each biospecimen used by the customer. In total, this information makes up the specimen management standards for that customer. By their presence, specimen management specification is formalized and made consistent from study to study, thereby reducing the potential for error and improving efficiencies for the customer, ultimately resulting in specification of the specimen management collection plan more quickly and with higher quality than would otherwise be possible.

The default operational details may include one or more of a default isolate indication of whether to isolate a portion of the specimen specific to an assay, a default store remainder indication specifying whether any remainder of the specimen after assay processing is to be stored, a default shipped from indication indicating a vendor from which the specimen is shipped, a default shipped matrix indication specifying a type of specimen, a default shipped medium indication specifying a medium for shipping, a default shipped to indication specifying where the specimen is to be shipped for purposes of assay processing, a default amount indication specifying an amount of the specimen required to process the assay, and a default unit indication specifying a unit for the amount of the specimen. As noted above, various fields for presenting the default operational details may be contingent on values entered into other fields for presenting the default operational details.

In any event, specimen management (SP) microservice 130B (which may be referred to as “SP microservice 130B”) may generate, responsive to priming the default operational details from standards maintained in the microservice, and receiving study-specific modifications to those standards via SMUI 131B, a plan defining a sequence of one or more events and a corresponding one or more timepoints during which the collection of the specimen occurs for the cohort of the subjects in the clinical study. Plan generation is initiated via SME through SMUI and is possible once the SOA is locked and all biospecimens for the study are completely specified (i.e., all required attributes of biospecimens, assays and operational details are populated). The plan may refer to a specimen collection plan for coordinating collection of the specimen during the events at the corresponding timepoints. SM microservice 130B may interface with SMUI 131B to present, via the SMUI 131B, the plan for modification, including defining override operational details that override the default operation details for the collection of the specimen.

The override operational details include one or more of an override isolate indication that overrides whether to isolate the specimen for the assay at the one or more events for the one or more corresponding timepoints, an override store remainder indication specifying an override for whether any remainder of the specimen is to be stored, an override cohort indication identifying an override cohort, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect. The override operational details may only override the default operational details for the a selected collection within a particular plan, where the overriding information is used to generate a modified collection for the plan with changes to a particular collection element of assays (where collection elements represent a collection or merger of different assays for one or more specimens during events at corresponding timepoints), underlying aliquots (which refer to a subset of the assays within the particular collection element, where the subset of assays collect the same specimen for the same cohort), and the like.

As noted above, SM microservice 130B may obtain the Schedule of Activities prior to being locked from further editing (or in other words, finalized), generating, from the possible Study Activities that are current present in the unfinished Schedule of Activities, the set of biospecimens required to support those activities, thereby allowing SM microservice 130B to generate default information for the underlying assays and operational details that are required to support specimen collection. SM microservice 130B may only, in some examples, obtain Study Activities that are required, prepopulating selection of Study Activities as described above, but allowing the SME, via SMUI 131B, to select additional Study Activities that are optional. SM microservice 130B may interface with Study Design microservice 126 to obtain these Study Activities forming the non-formalized Study of Activities (via the subscribe and pull mechanism), and prepopulating at least a portion of the default operational details (which may also be referred to as vendor details) based on vendor requirements as recorded in biospecimen standards maintained by the microservice (e.g., central lab requirements that may vary depending on specimen processing capabilities, site specific requirements for collecting the specimen, etc.). The SME may modify these default operational details to change how specimen management occurs for the entire Schedule of Activities (corresponding to the Trial Arm) for the study under development, including adjustment of specimen collection and intermediate processing (i.e., aliquoting) steps leading to production of the required testing matrix (e.g., extraction of plasma from whole blood, conversion of an extracted tissue sample from raw tissue to a preserved block to slices mounted on laboratory slides).

In this way, the techniques of the disclosure may organize specimen management within the digital framework provided by clinical research study system 100 in a manner that allows concurrent specimen management specification during study design, while still potentially providing flexibility to allow the SME to tailor (via the above noted override, split, and merge) specimen collection specification for particular events. Through organized specimen management standards and structured SME tailoring of those standards for a specific study, specimen management microservice 130B may produce compact specimen collection plans to allay subject burden (e.g., in terms of time spent undergoing specimen collection, amounts of specimen collection, visits/event frequency, etc.) while still enabling SMEs to accommodate incomplete and often times flexible specimen management standards. Moreover, system 100 may specify a single vendor twice in the vendor details for a specific assay so that there can be different rules for different grouping of subjects (which is another way to refer to cohorts), or to specify that a particular assay be processed multiple times (e.g., to provide redundancy in producing assay results because processing of that assay is sensitive). The SME may provide this information to refine or specialize the standards information for this clinical study, thereby defining default operational details for the assays that are used in the aforementioned collection plan generation for the study.

System 100 shown in the example of FIG. 1 may be implemented on one or more computing devices that comprise processing circuitry and a memory. In some examples, the processing circuitry is one or more microprocessors, digital signal processors (DSPs), application specific integrated circuits (ASICs), field programmable gate arrays (FPGAs), or any other equivalent integrated or discrete logic circuitry, as well as any combinations of such components. In some examples, the memory is random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Further, this memory may be implanted entirely in hardware, software, or a combination thereof.

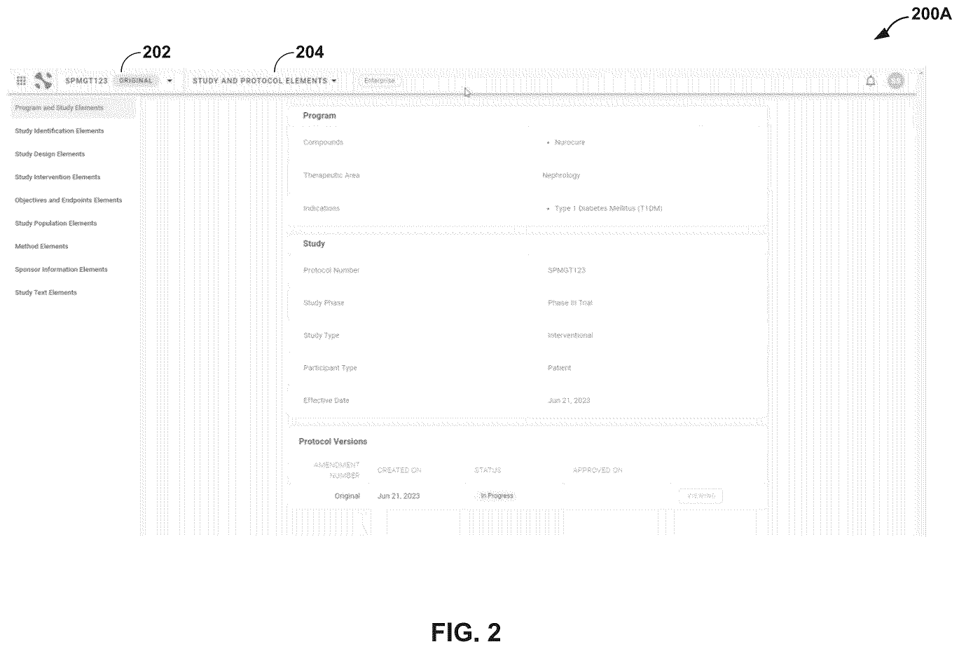

FIGS. 2-23 illustrate example user interfaces presented by the clinical research study system shown in the example of FIG. 1 in performing specimen management in accordance with various aspects of the techniques described in this disclosure. Referring first the example of FIG. 2, study design microservice 126 (shown in the example of FIG. 1) may interface with study and protocol elements/disclosure 116 to present study and protocol elements user interface 200A. Study and protocol elements user interface 200A may enable the user to define a program and a study, while managing protocol versions. Within study and protocol elements user interface 200A, the user may navigate between (as shown in the right of study and protocol elements user interface 200A) different tabs entitled “Program and Study Elements,” “Study Identification Elements,” “Study Design Elements,” “Study Intervention Elements,” “Objectives and Endpoints Elements,” “Study Population Elements,” “Method Elements,” “Sponsor Information Elements,” and “Study Text Elements.” The user may interface with these study and protocol elements tabs to define the above noted elements of the protocol.

Study and protocol elements user interface 200A may include a study protocol dropdown box 202 and application (or in other words, user interface) dropdown box 204 to interface with different user interfaces of UI framework 102. Currently, the user has selected the “Study and Protocol Elements” application from application dropdown box 204, which allows the user to specify “Program and Study Elements” in which the Program and Study Elements are shown in the main pane of study and protocol elements user interface 200A.

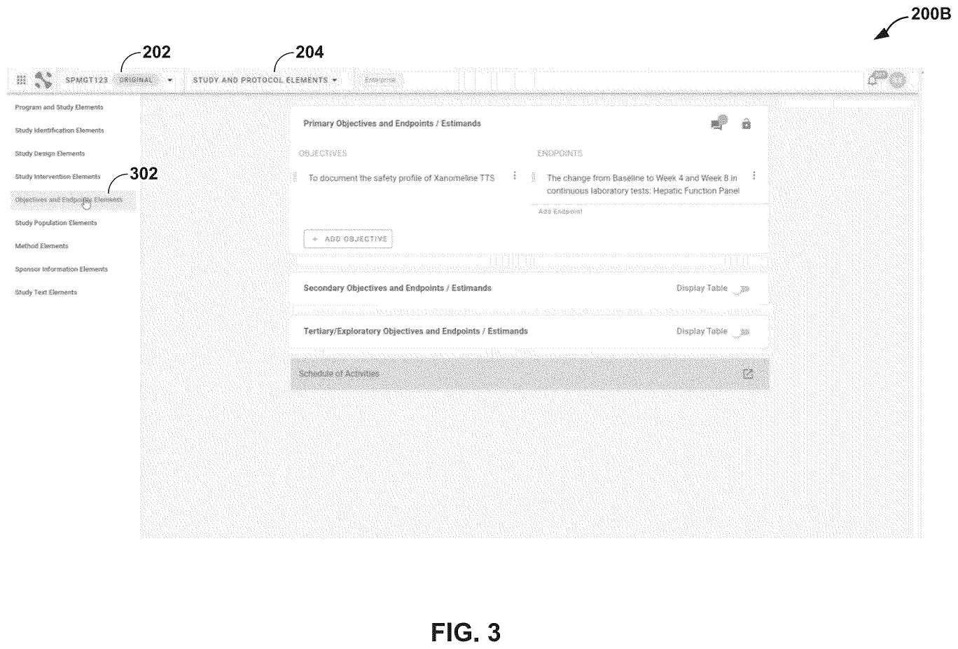

In the example of FIG. 3, the user has selected tab 302 entitled “Objectives and Endpoint Elements,” causing study and protocol elements user interface 200A to transition the main pane to show the primary objections and endpoints/estimands in interface 200B. Endpoints, as noted above, represent how to prove out the scientific hypotheses. In response to populating the endpoint, study design microservice service 126 may determine, based on the endpoint, an activity and populate the schedule of activities.

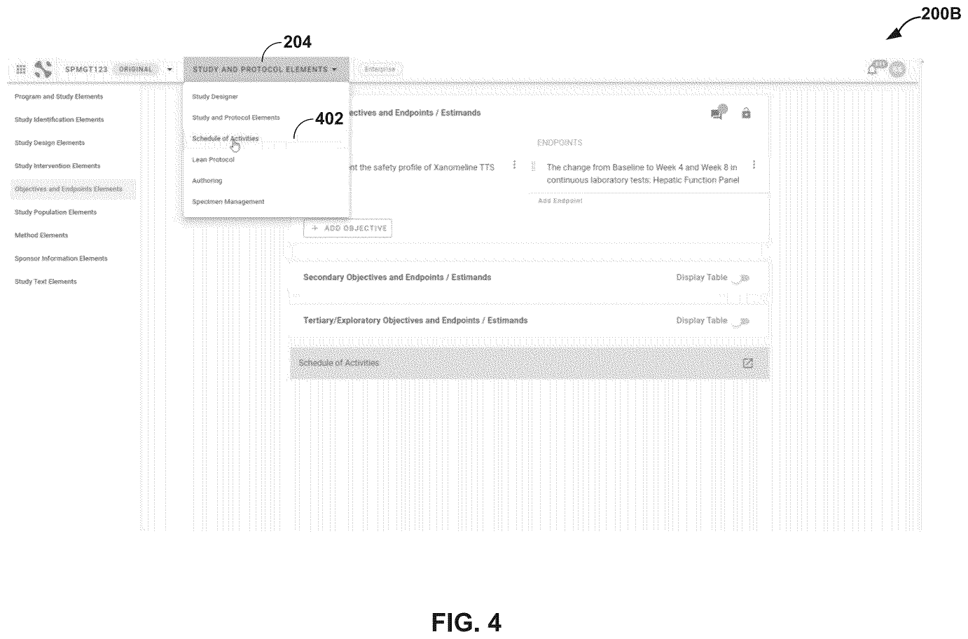

In the example of FIG. 4, the user has selected application dropdown box 204 to expose application elements entitled “Study Designer” (which invokes GSD 150), “Study and Protocol Elements” (which invokes Study and Protocol Elements/Disclosure 116), “Schedule of Activities” (which invokes Schedule of Activities 114), “Lean Protocol” (which invokes LPUI 131A), “Authoring” (which invokes Protocol Authoring 118), and “Specimen Management” (which invokes SMUI 131B). In some examples, the user may view more or less application elements depending on the user permissions assigned to the user during authentication of the user to access system 100. The user may then select application element 402 entitled “Schedule of Activities,” which is shown in interface 200B and thereby invokes Schedule of Activity 114.

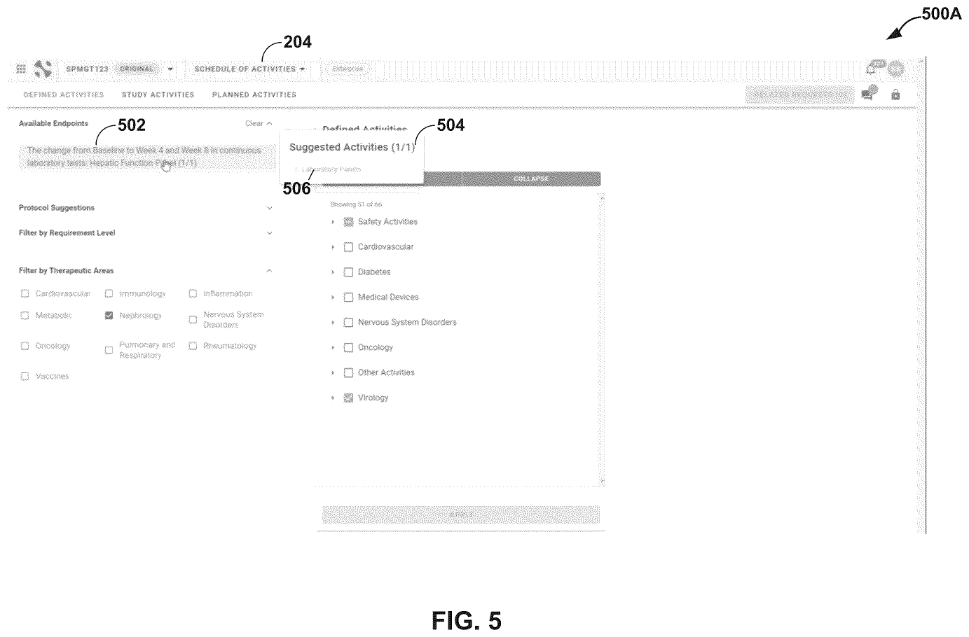

Referring next to the example of FIG. 5, Schedule of Activity 114 may present schedule of activity (SoA) user interface 500A whereupon the user may select Available Endpoint 502 to expose Suggested Activities list 504 suggesting that a Laboratory Panels Activity 506 is available for Available Endpoint 502. Schedule of Activity 114 may have this as an optional Activity that was added by way of study design microservice service 126. SoA user interface 500A includes a main pane that specifies “Defined Activities” (which is partially covered by Suggested Activities list 504). The Defined Activities, although not expanded, would not indicate that a possible Laboratory Panels Activity 506 has been formally added in Schedule of Activity 114.

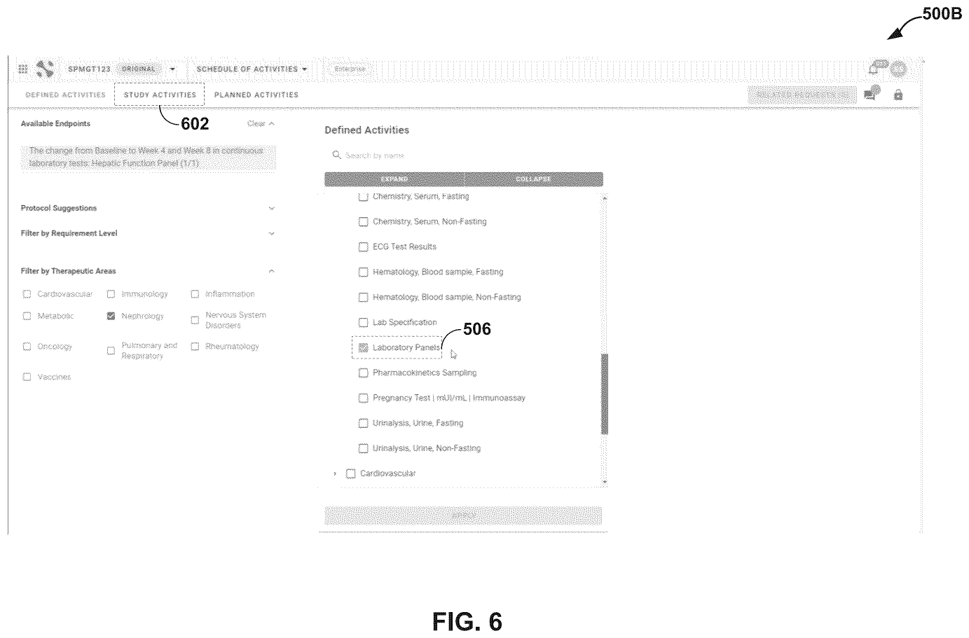

Assuming the user selects Laboratory Panels Activity 506, Schedule of Activity 114 may present SoA user interface 500B shown in the example of FIG. 6, where the user can expand the “Safety Activities” Defined Activity category and scroll to identify that Laboratory Panel Activity 506 has been selected. The user may next select Study Activities tab 602.

It should be understood that the user selecting any element results in the underlying application, i.e., Schedule of Activity 114 in the example of FIG. 6, sending the selection to an underlying microservice, i.e., study design microservice 126 in the example of FIG. 6, which receives this input (or data indicative of an input, which may represent a selection of a user interface element), processes the input and potentially passes an output that is processed by some component of system 100 (such as Schedule of Activity 114 in this example to transition between different user interfaces).

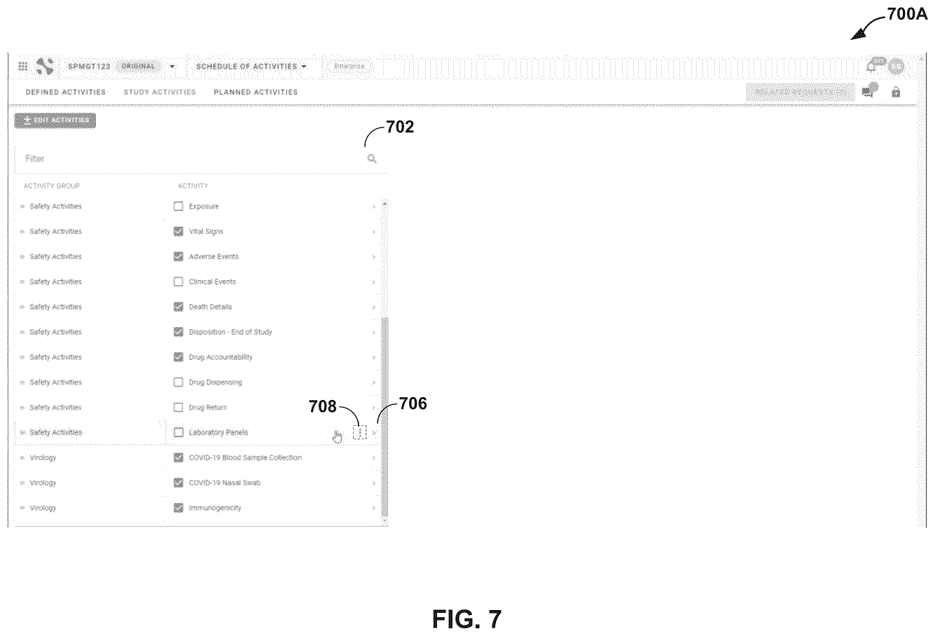

In any event, responsive to the user selecting Study Activity tab 602, Schedule of Activities 114 may present study activities user interface 700A (FIG. 7), whereupon the user may view all available Study Activities via a list 702 of study activities user interface 700A, scrolling through list 702 to identify Laboratory Panels list element 706 (that corresponds to Laboratory Panels Activity 506) in list 702. Laboratory Panels list element 706 may include additional options element 708 denoted by three vertical dots.

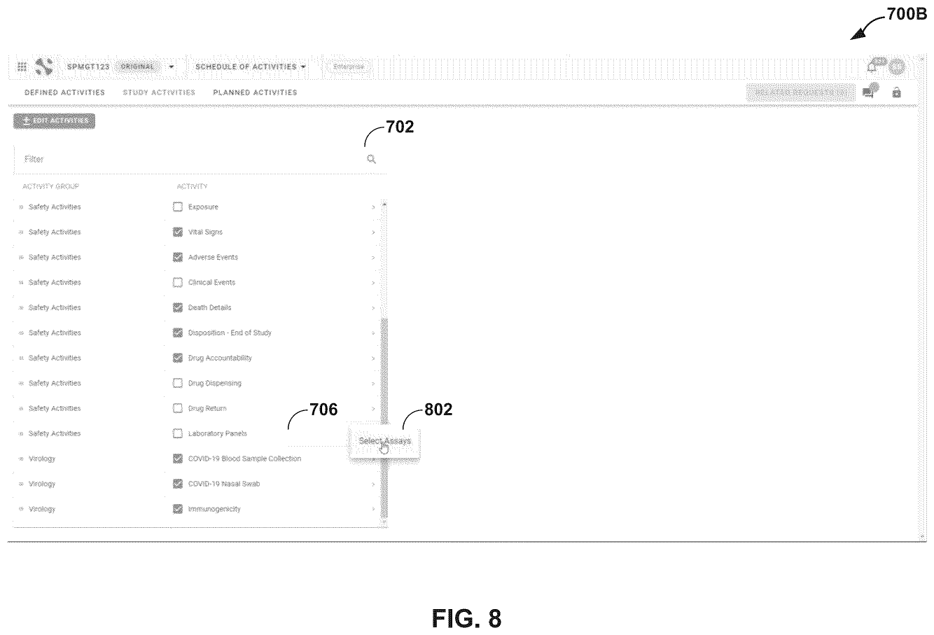

Responsive to the user selecting (or possibly hovering a cursor over additional options elements 708), study activities user interface 700A may transition to a study activities user interface 700B shown in the example of FIG. 8. Study activities user interface 700B may present an additional option as a Select Assays element 802, which may enable the user to select which Assays (or, in other words, laboratory panel tests) are to be performed for the Laboratory Panels list element 706.

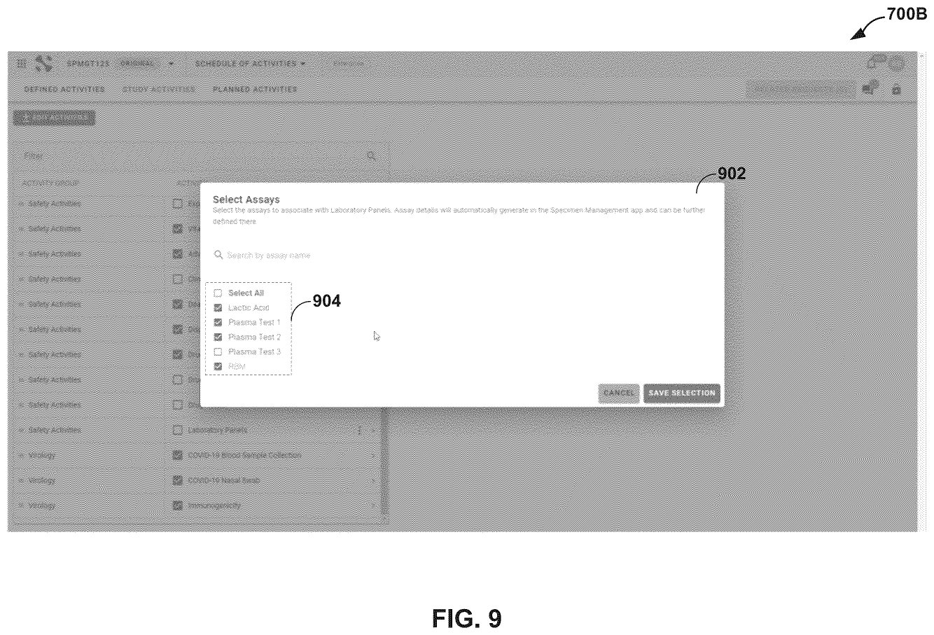

Responsive to the user selecting Select Assays element 802, study activities user interface 700B may present Select Assays popup window 902 shown in the example of FIG. 9. Select Assays popup window 902 may allow the user to select assays to include in Laboratory Panels Activity 506 (as represented by Laboratory Panels list element 706) via selection box elements 904. Selection box elements 904 includes a “Select All” selection box, a “Lactic Acid” selection box, a “Plasma Test 1” selection box, a “Plasma Test 2” selection box, a “Plasma Test 3” selection box, and a “RBM” selection box. The user, which may be an SME, may also save the selection (via “SAVE SELECTION” element) or cancel out of Select Assays (via “CANCEL” element).

SoA user interface 700B may populate selection boxes from information coming from the specimen management standards. For example, SoA user interface 700B may determine (possibly via interations with other components of system 100), for that specimen (which may also be referred to as a “biospecimen”), and via a pharmaceutical organization, that typical Assays represented as selection box elements 904 are generally conducted for a particular specimen, e.g., blood. The SME may determine that, for this particular study, only four of five Assays are required, allowing the SME to refine what the standard says for the clinical study.

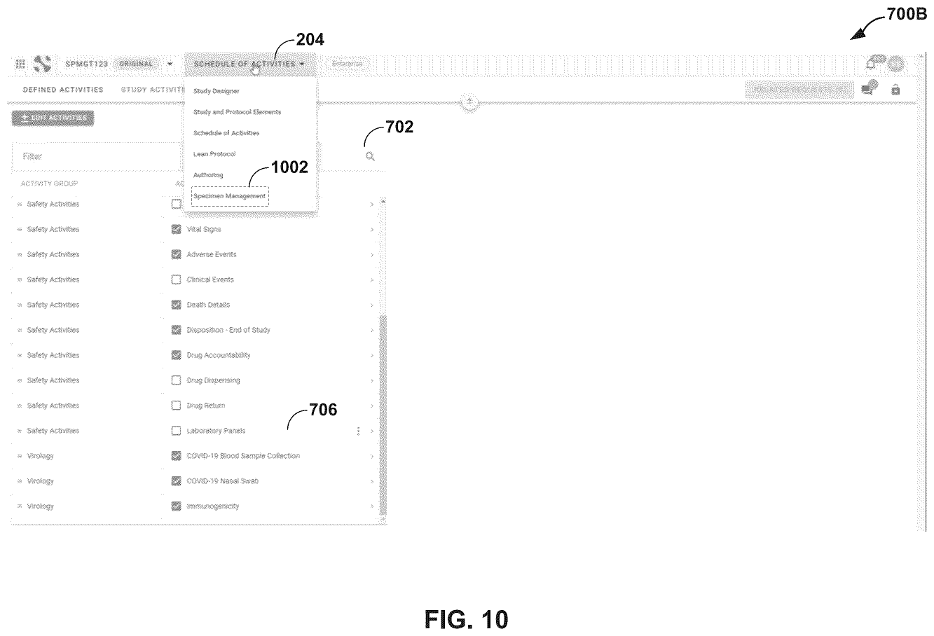

Responsive to the SME saving the selection shown in selection box elements 904, SoA user interface 700B may remove Select Assays popup window 902, whereupon the user may select application dropdown box 204 and select Specimen Management dropdown box element 1002, which invokes SMUI 131B (FIG. 10).

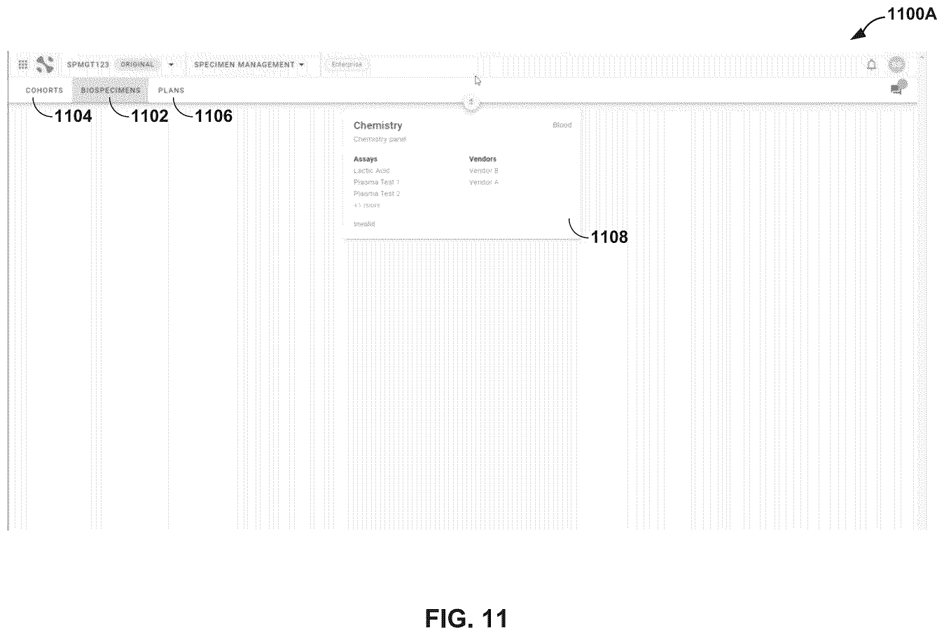

SMUI 131B may present biospecimen user interface 1100A shown in the example of FIG. 11. Biospecimen user interface 1100A may include tabs 1102-1106 and card element 1108. Tab 1102 (which is the active tab as denoted by the shading) may list, as card elements (such as card element 1108), one or more biospecimens. Tab 1104 may transition biospecimen user interface 1100A to a cohort user interface, while tab 1106 may transition biospecimen user interface 1100A to a plan user interface. Card element 1108 may represent a biospecimen upon which one or more assays (e.g., the four assays selected for the Laboratory Panels list elements 706) are performed by one or more vendors. Card element 1108 indicates that chemistry assays are selected for a specimen type denoted as “Blood.”

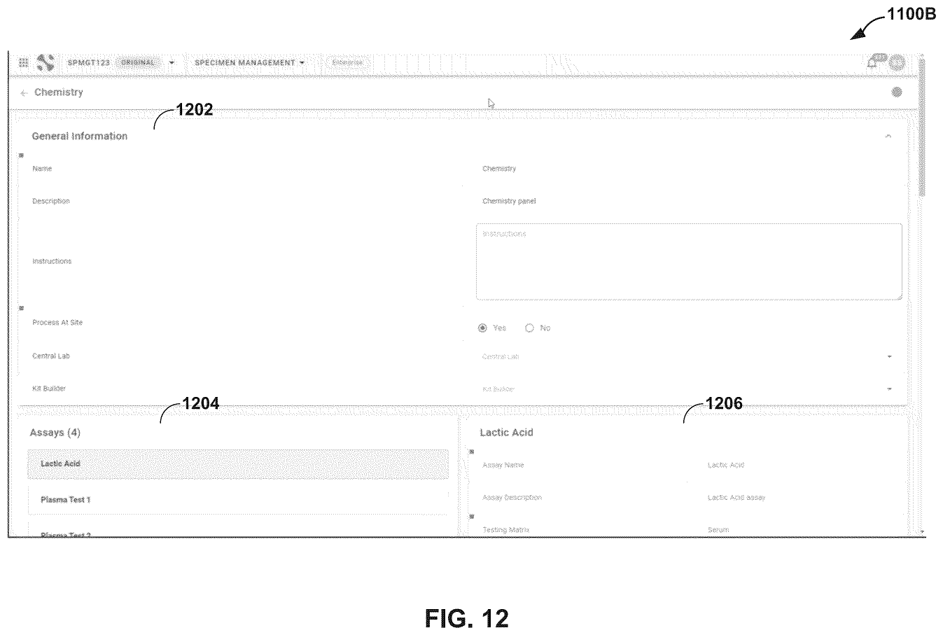

Responsive to the user selecting card element 1108, biospecimen user interface 1100A may transition to biospecimen user interface 1100B (FIG. 12). Biospecimen user interface 1100A may include a general frame 1202 listing general information for all of the assays defined in assays frame 1204. Assays frame 1204 may list the four assays noted above, which the user may select to expose assay information frame 1206. The user may specify default operational details for each of the assays via assay information frame 1206.

General frame 1202 may allow the user to specify a name for the grouping of assays, a description of the grouping of assays, general instructions for the grouping of assays, whether specimens from the grouping of assays are processed at the collection site or at a central lab, (when such specimens are processed at the central lab) a central lab to use when processing the specimens for the grouping of assays, and a kit builder responsible for building the specimen collection kit that may be sent to the collection site for collecting the specimens and sending them to the central or other labs as specified (where the kit builder may be different than the central lab, but may also be the same as the central lab).

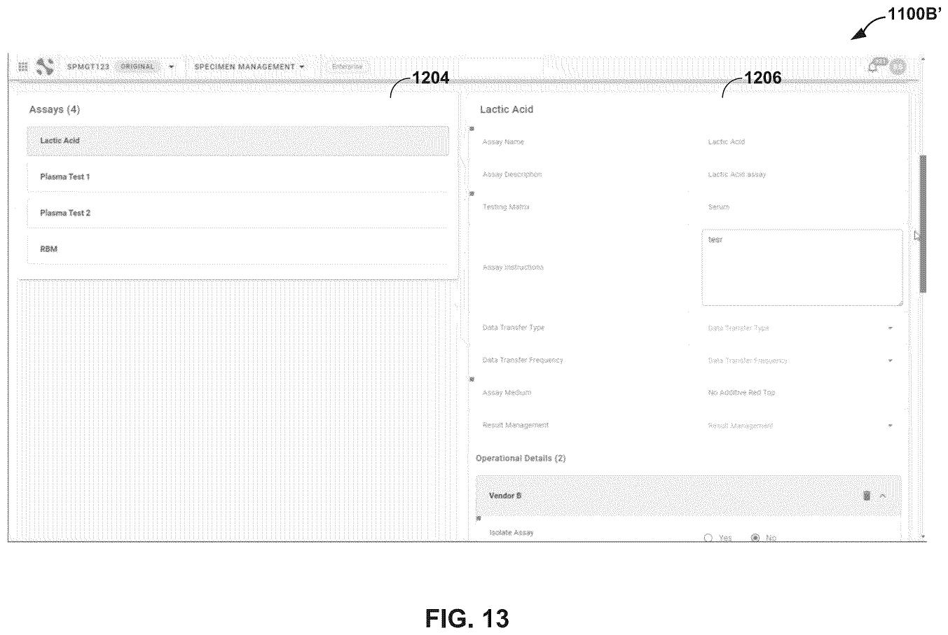

The user may scroll biospecimen user interface 1100B to more fully view assay frame 1204 and assay information frame 1206, resulting in biospecimen user interface 1100B′ shown in the example of FIG. 13. As shown in the example of FIG. 13, assay information frame 1206 includes an assay name field for assigning a name to the assay, an assay description field for providing a description of the assay, a testing matrix field for specifying the specimen to be collected for the assay, an assay instructions field for specifying instructions for performing the assay, a data transfer type for defining how data is to be transferred in support of conducting the assay, a data transfer frequency for specifying how frequently the data transfer occurs, an assay medium field for specifying how the specimen is prepared (e.g., no additive and a type of container for the specimen, e.g., red top container, in this instance), a result management field for specifying how results—meaning data—from the assay are managed (meaning, what entity is going to process the data from the assay).

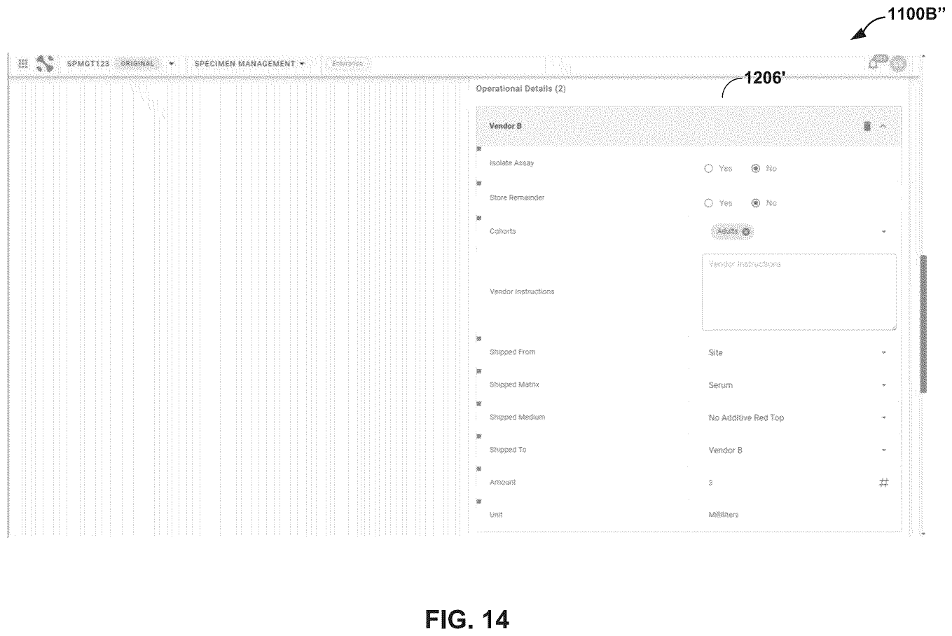

Biospecimen user interface 1100B′ may, in this way, link assay details at an assay level of granularity to the standard. As such, biospecimen user interface 1100B′ may drive operational details (which again may be referred to as “vendor details”) at the assay level and define the operational details from the standard. The user may continue to scroll biospecimen user interface 1100B′ to expose the operational details associated with the assay selected in assay frame 1204 (which in this example is the “Lactic Acid” assay) to expose biospecimen user interface 1100B″ shown in the example of FIG. 14.

Biospecimen user interface 1100B″ may include assay information frame 1206′ (which is the scrolled version of assay information frame 1206), whereby the user may specify operational details for the assay selected in assay frame 1204. In this example, there are two sets of operational details, one for each of the two different cohorts of “Adults” and “Children.” The operational details for the adults cohort is shown in assay information frame 1206′, which includes an isolate assay field for specifying whether or not to isolate the assay, a store remainder field for specifying whether any remainder after processing the specimen is to be stored or not, a cohort field for specifying one of the available cohorts (in this instance the “Adults” cohort), a vendor instructions field for specifying instructions to the vendor processing the specimen, a shipped from field for specifying where the specimen is shipped from (i.e., from the collection “Site” in this example), a shipped matrix for specifying the specimen shipped, a shipped medium for specifying how the specimen is prepared, a shipped to field for specifying where the specimen is to be shipped to, an amount field for specifying an amount of specimen shipped to the vendor, and a unit field for specifying units used in the amount field.

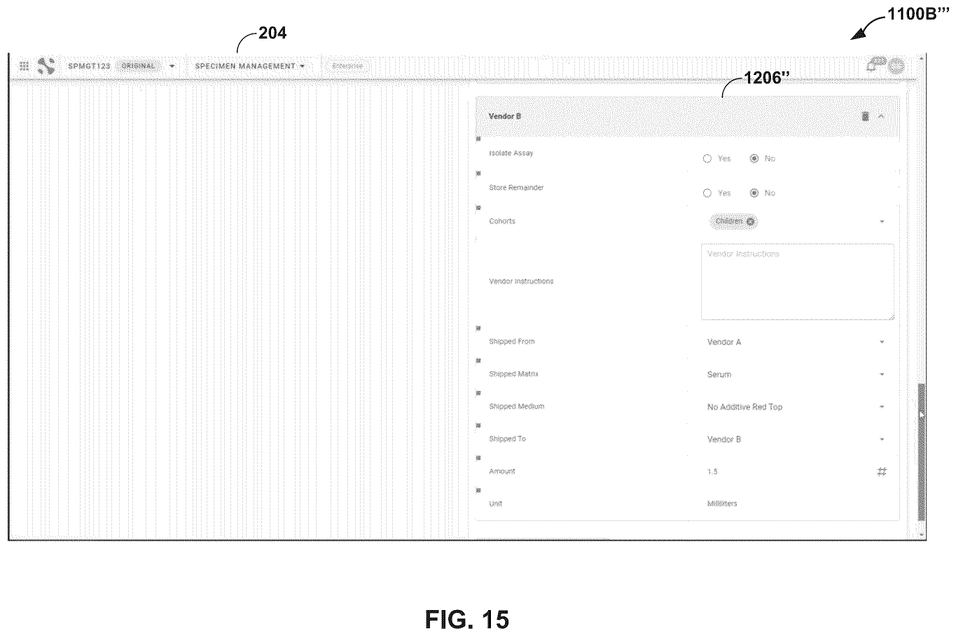

The user may continue to scroll biospecimen user interface 1100B″ to expose biospecimen user interface 1100B′″ that includes assay information field 1206″ (FIG. 15). Assay information field 1206″ includes the same fields as discussed above with respect to assay information field 1206′, but is separate from assay information field 1206′ due to there being multiple cohorts. Multiple cohorts may result in biospecimen user interface 1100B (which may refer to the entirety of biospecimen user interface 1100B in this instance) specifying different operational details for each of the cohorts. In this instance, because the “Children” cohort may impact an amount of blood drawn (given that adults have more unit volume of blood than children), the user may specify a lower amount of blood drawn (1.5 milliliters) compared to the adult cohort in which a higher amount of blood is drawn (3 milliliters). In some instances the user may choose to omit the assay for a cohort (again because of blood volume restrictions); in this case a simple Omit Assay panel will be presented which will then allow the user to specify the cohort or cohorts for which the assay is to be omitted, and by extension the biospecimen collection amount reduced for that cohort or cohorts.

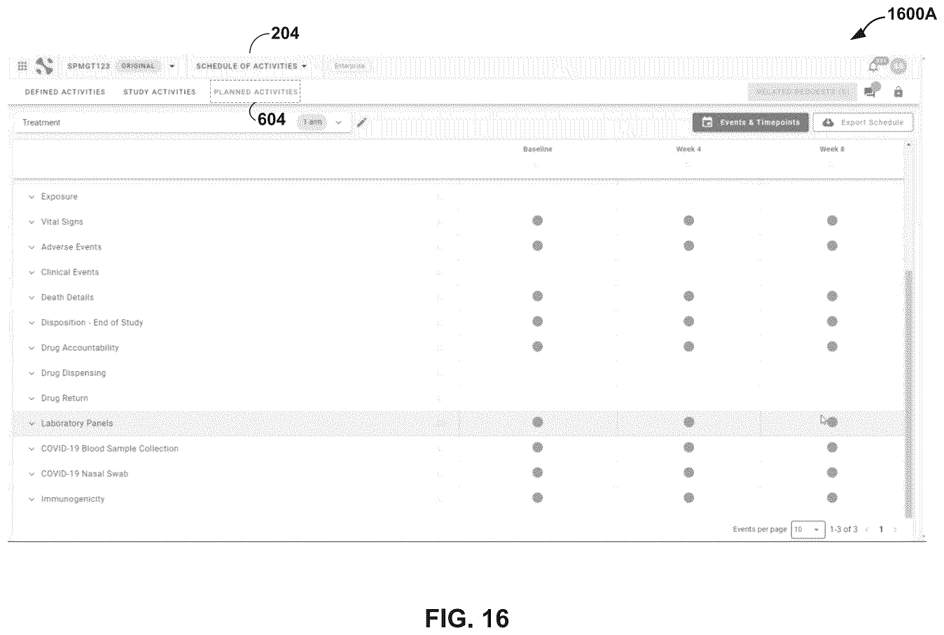

After specifying or otherwise defining the default operational details, the SME may interface with biospecimen user interface 1100B″ to select, via dropdown box 204, Schedule of Activities 114 to present (after selecting tab 604 entitled “PLANNED ACTIVITIES” and planned activities user interface 1600A (after the user has scrolled down to expose the “Laboratory Panels” Activity) shown in the example of FIG. 16. The plan may show events and corresponding activities, denoting events for a corresponding timepoint currently assigned to the plan by filled circles. The user (which in this instance may refer to an SME) may interface with planned activities user interface 1600A to deselect or select events for each corresponding timepoint to add and/or remove events from the plan.

In the example of FIG. 16, planned activities user interface 1600A may indicate that the Laboratory Panels Activity is scheduled for Baseline, Week 4, and Week 8. In other words, planned activities user interface 1600A may indicate, for the at least one biospecimen activity (“Laboratory Panels” Activity) that there are three events, and because the SME specifying biospecimen collection identically for the three events, system 100 may sort these three events by one plan, which reduces data entry given that the assays were defined a single time and populated across three events rather than requiring separate entry of the assay operational details for each of the three events (as is commonly performed, even by central labs, collection sites, study developers, etc.).

In one example, the SME may determine that the “COVID-19 Blood Sample Collection” is not required for the baseline event, deselecting the corresponding full circle for that event. In this configuration, system 100 may generate two plans (since they are not identical visits) with a first plan dedicated to Baseline and a second plan that shared Week 4 and Week 8. In this example, system 100 may combine Laboratory Panel collection with COVID-19 Blood Sample Collection into a single biospecimen collection for Weeks 4 and 8 (but possibly not given other consideration, such as required blood draw volume units to perform the combined tests). Combining biospecimen collection across activities may depend, as another example, on whether the Activities being combined involve the same testing activity or if the substance/specimen to be tested is the same. If for some reason, the SME decides not to combine Activities, the SME may disable the combination of the Activities by decoupling one or more of the events away from the single default compact plan (via Collection Groups). In this way, system 100 attempts to generate the most compact plan possible, but allows the SME to tailor the plan to be whatever is required and/or better (in their opinion).

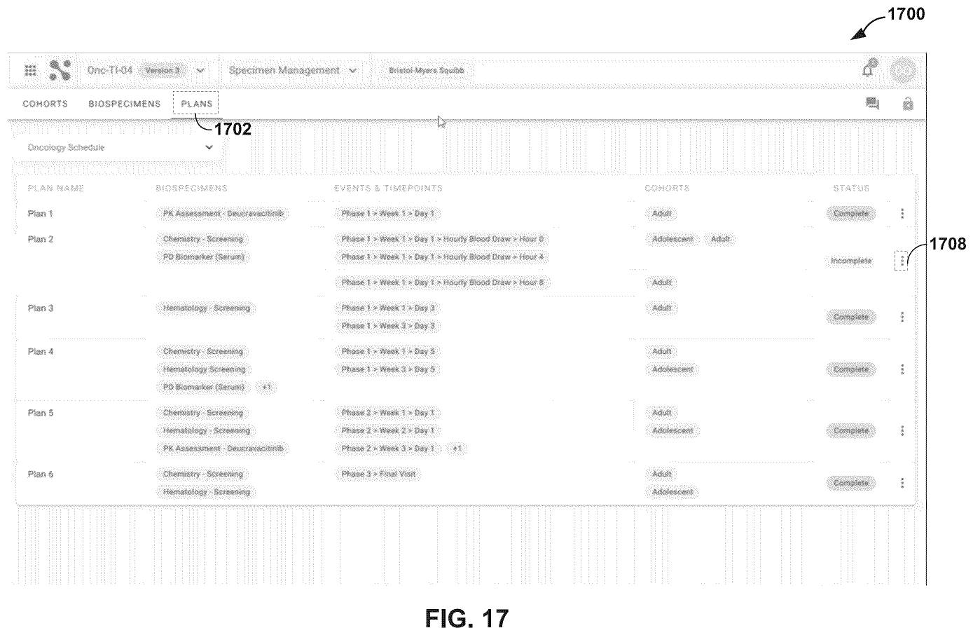

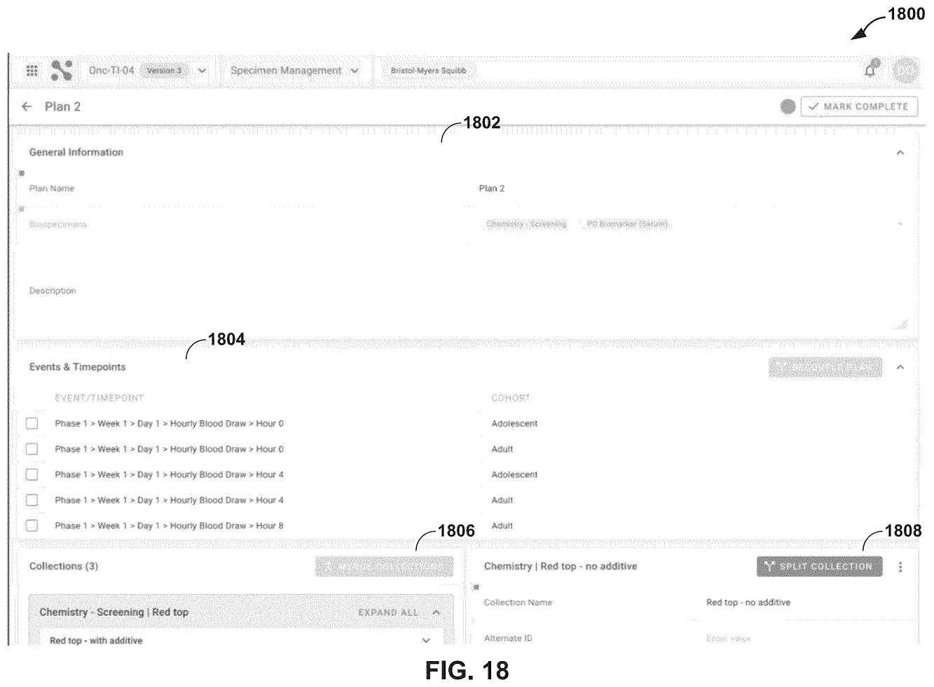

Once the planned activities are locked by the SME (and possibly dependent upon other users completing different portions that support the plan), the SME may interface with application dropdown box 204 to select the “Specimen Management” application, invoking SMUI 131B. SMUI 131B may present plans specimen management user interface 1700 shown in the example of FIG. 17 (after the SME selects tab 1702 entitled “PLANS”). Plans specimen management user interface 1700 presents plans 1-6 in terms of biospecimens collected, corresponding events and timepoints, cohorts, and status (whether the plan is complete or incomplete). The SME may interface with plans specimen management user interface 1700 to select one or more plans for modification. In this example, it is assumed that the SME selects incomplete plan 2 for modification (possibly by selecting an additional option element 1708 to expose a popup window and then selecting “Modify Plan” within the popup window), whereupon SMUI 131B may present plan modification user interface 1800 shown in the example of FIG. 18.

Plan modification user interface 1800 may enable the SME to modify the generated plan, which may include specifying override operational details for the Assays in accordance with various aspects of the techniques described in this disclosure. Plan modification user interface 1800 may include a general information frame 1802, an events and timepoints frame 1804, a collections frame 1806, and a collection information frame 1808.

General information frame 1802 may include a plan name field for specifying a name for the selected plan undergoing modification, a read-only biospecimens field for specifying the specimens to be collected for the plan (which may be locked given the Schedule of Activities is locked; hence the inactive—gray out shading), and a description field for specifying a description for the plan undergoing modification. Event and timepoints frame 1804 may include event/timepoint for each activity in the plan undergoing modification in terms of the applicable cohort for each event/timepoint linked to each activity. By selecting one or more items in frame 1804, the SME may choose to decouple the single compact plan generated by default into two separate plans, with the second plan being assigned the selected items. This allows the SME to adjust for specific events/timepoints/cohorts combinations away from the default generated plan to a configuration more suited for the study (in the opinion of the SME).

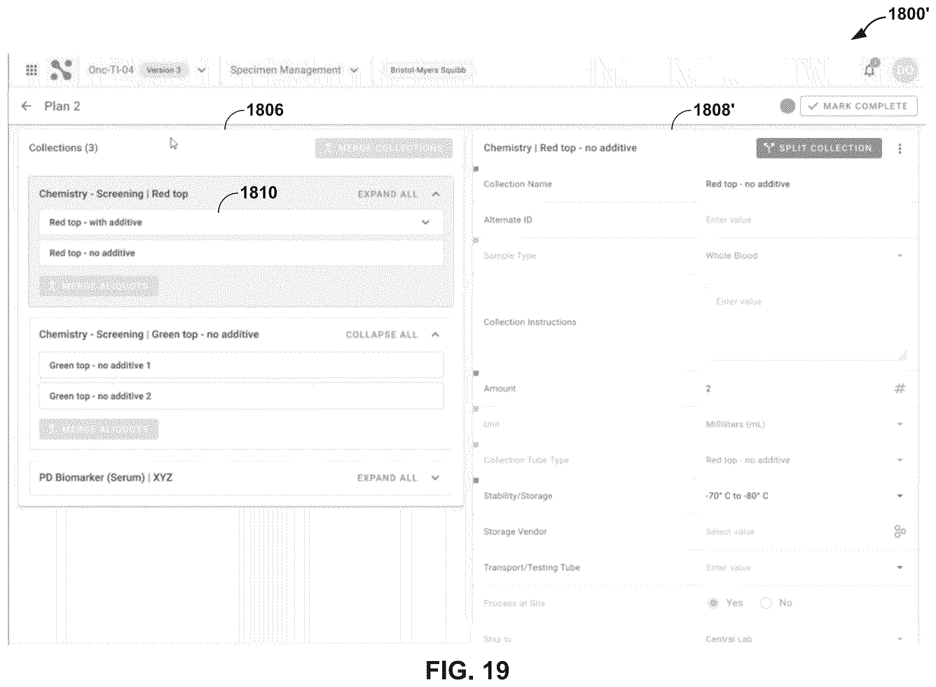

Collection frame 1806 may present a collection in support of assays that were previously combined (or in other words compacted into a single biospecimen collection), where subsets of combined biospecimens are presented as aliquots (where it should be understood that subset is again not used in the strict mathematical sense, but refers to one or more, but not all, elements of the Assay set and excludes all elements of the Assay set and zero elements of the Assay set). Aliquots represent different processing paths for the biospecimen originally collected at the study site, where those processing paths are determined by the study-specific attributes specified for the biospecimens, their assays and operational details that have been combined into the collection. Collection information frame 1808 may present relevant information for the selected collection in collection frame 1806. The SME may scroll down within plan modification user interface 1800 to expose plan modification user interface 1800′ shown in the example of FIG. 19.

Plan modification user interface 1800′ presents collection frame 1806 in its entirety and a portion of collection information frame 1808 denoted collection information frame 1808′. Collection frame 1806 presents three (3) different collections. Each of the collections are separate because the underlying Assays require collecting different bodily substances (which is an example of specimens) or require collecting the same bodily substances in different ways (where, as one example, blood may be deposited in a different medium to produce plasma compared to the medium used when producing serum).

Collections are organized as aliquots (which represent a processing step of the collected biospecimen such as transformation to a different matrix (i.e., physical form), subdivision to support multiple transportation and processing paths, etc.) and assays, where system 100 may manage collections via a tree data structure (or other data structure, such as a linked list, graph, etc.). In this respect, system 100 may start at the assay level (which may be a leaf node in the constructed tree data structure) and moving upwards from these leaf nodes representative of the assays to a root node representative of the collection. System 100 may attempt to construct the most compact (in terms of number of nodes) tree data structure through combining of assays into a single node forming an aliquot (meaning the two or more leaf nodes having the same specimen and the same preparation) can be combined. There may be layers of aliquots despite there being a single aliquot shown for the collections in plan modification user interface 1800′. Although shown as a nested hierarchy of collections, aliquots, and assays (although not shown in the example of FIG. 19), collection frame 1806 may represent the hierarchy in other formats, such as a graphical representation of the tree data structure. Each path through the tree data structure may be considered a “sample journey” depicting how the sample (or, in other words, specimen) is processed during the plan.

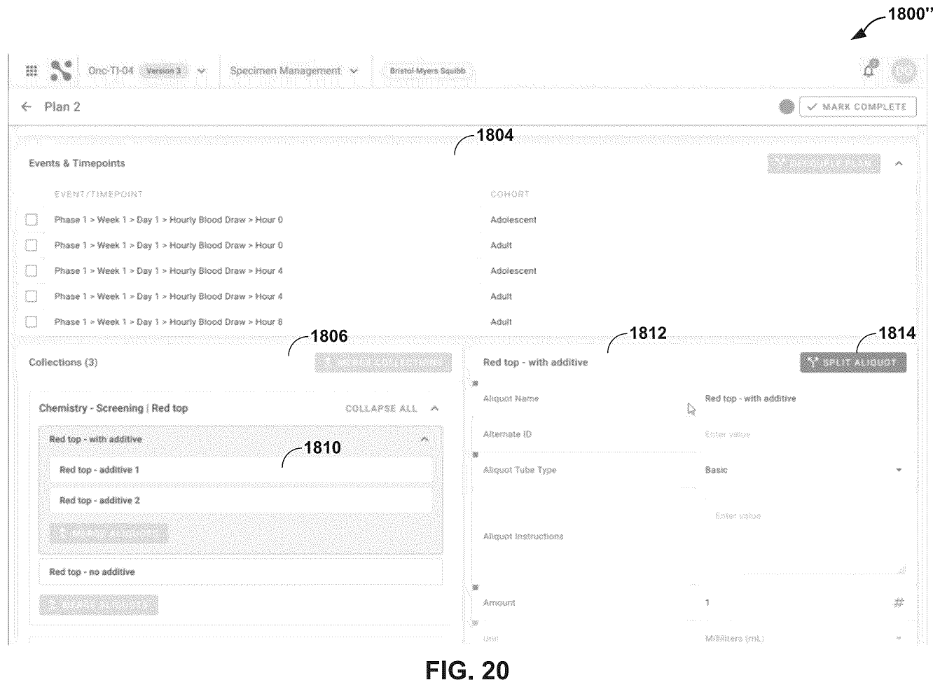

The SME may select an aliquot, such as an aliquot 1810 that selects a Chemistry (blood type) aliquot with a storage medium of “Red top-with additive”), whereupon plan modification user interface 1800′ may transition to a plan modification user interface 1800″ shown in the example of FIG. 20. Plan modification user interface 1800″ includes an aliquot information frame 1812 providing aliquot attributes (which are a subset of the operational details) for review and/or modification by the SME similar to the operational details for collection information frame 1808/1808′. While described with respect to a split aliquot, various aspects of the techniques may allow for system 100 to split collections that are necessary to support assays, which is shown above in collection information frame 1808/1808′.

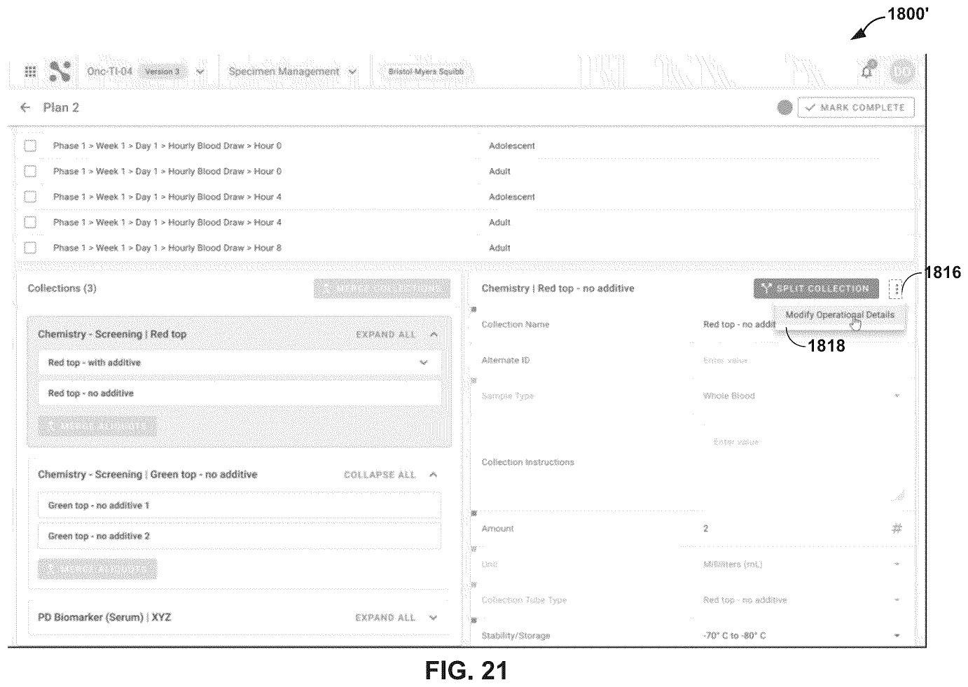

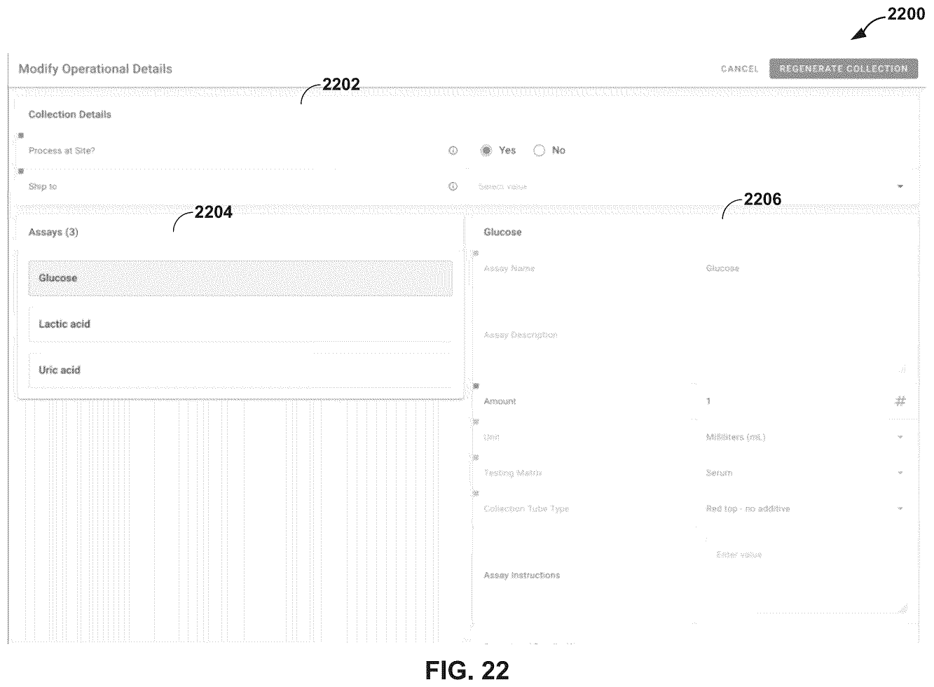

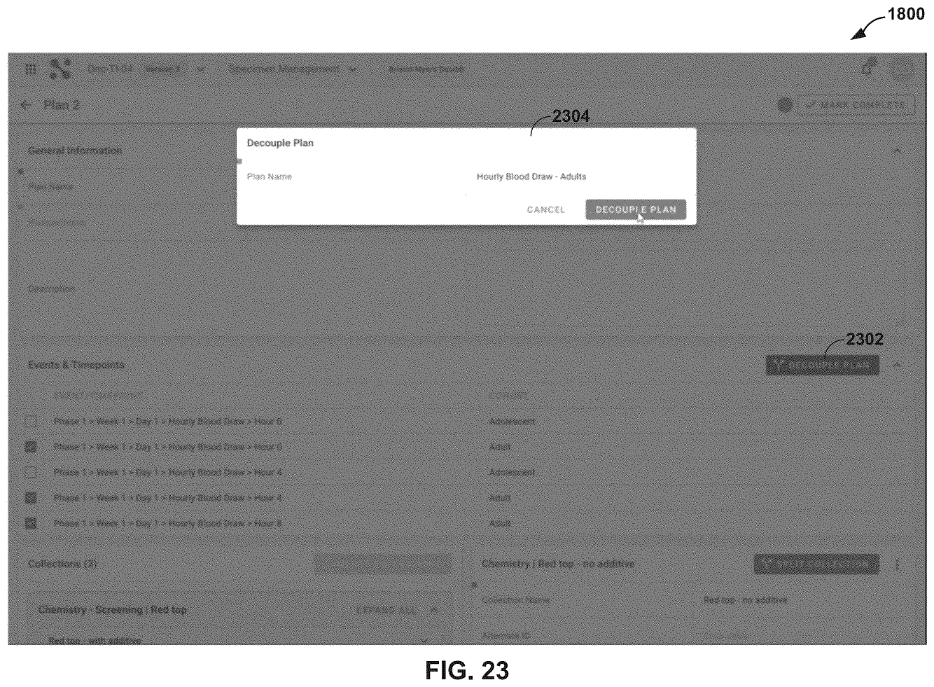

The SME may interface with plan modification user interface 1800″ to return to plan modification user interface 1800′ and select an additional details element 1816 to expose modify operational details pane 1818, as shown in the example of FIG. 21. The SME may select the modify operational details pane 181 to expose collection operational details user interface 2200 shown in the example of FIG. 22. Collection operational details user interface 2200 includes a collection details pane 2202 in which the SME may specify operational details for the collection (which may override the default operational details derived from plan generation and discussed above and therefore represent override operational details), an assays pane 2204 listing all of the assays associated with the selected collection, and an assays details pane 2206 in which the SME may review and/or modify operational details associated with the assay selected in assays pane 2204 (and thereby specify override operational details that override the default operational details). The SME may also interface with a plan decouple element 2302 of plan modification user interface 1800 shown in the example of FIG. 23 to decouple plans that have been previously compacted, while also (after decoupling) rejoining plans that have been previous decoupled via a rejoin plan element that is similar to decouple plan element 2302.

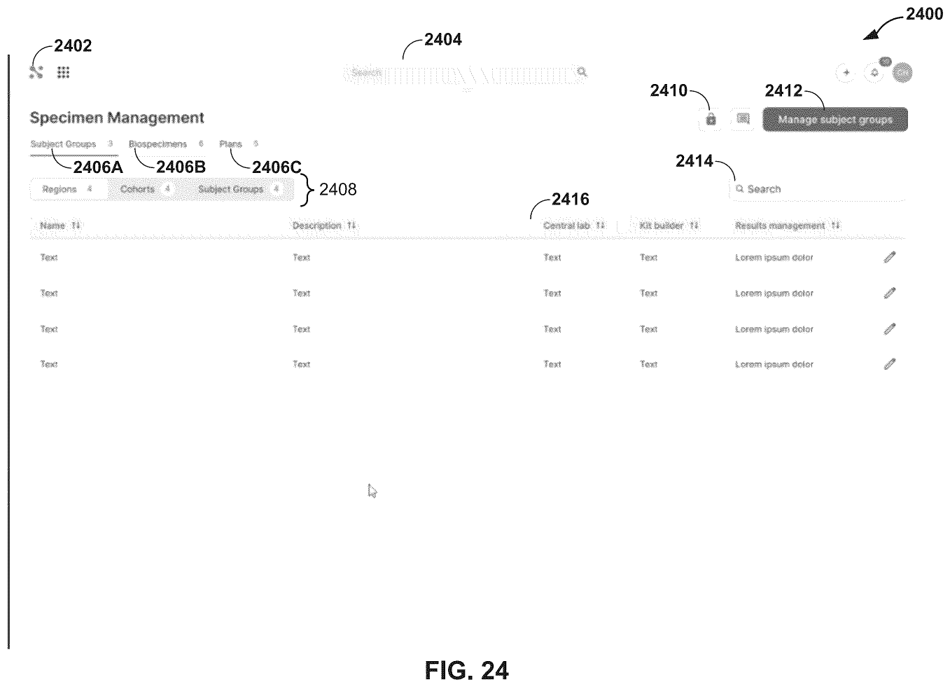

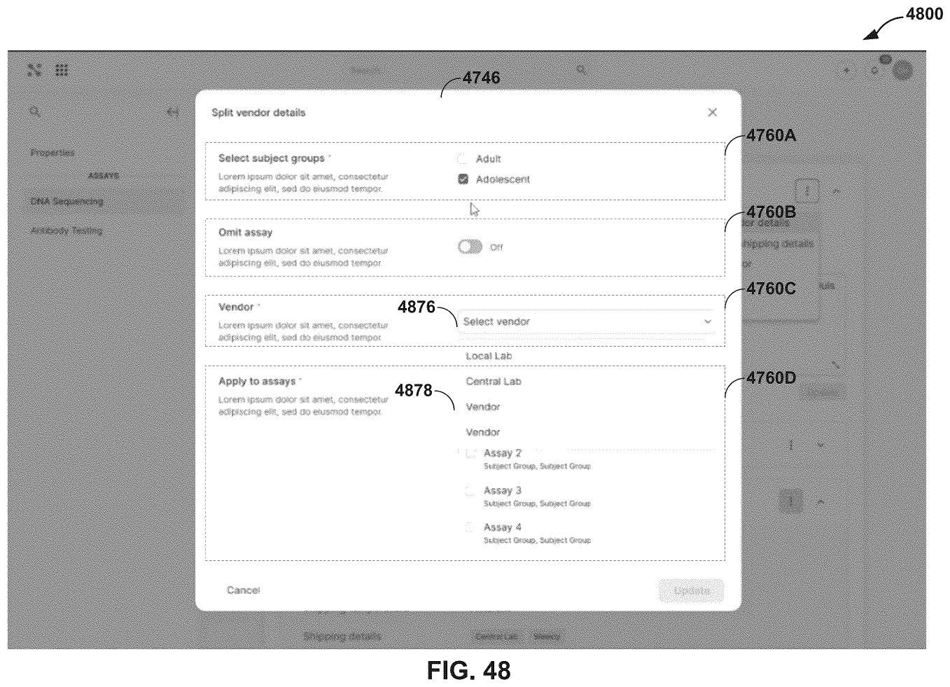

FIGS. 24-57 illustrate another example set of user interfaces presented by the clinical research study system shown in the example of FIG. 1 in performing specimen management in accordance with various aspects of the techniques described in this disclosure. It should be understood that one or more of user interfaces 2400-5700 shown in the example of FIGS. 24-57 may replace and/or be added in addition to one or more of user interfaces 200A-2200 shown in the examples of FIGS. 2-23.

In any event, the object model present in user interfaces 200A-2200 shown in the examples of FIGS. 2-23 may promote some amount of confusion in that the collected or subsequently processed i.e., aliquoted specimen or substance is not clearly distinguished from the routing path that the collection or aliquot went through (to whom the collection or aliquot is shipped, what assays are being processed, etc.). This lack of separation may result in issues with being able to clearly identify what is being done over the life of the collection (which may also be referred to as a “specimen journey”). A specimen journey may specify what happened to the specimen itself and where those results actually travel or are stored. For example, a specimen journey for blood may indicate that the blood is processed into serum (e.g., where the specimen is being shipped, how the specimen is being stored, etc.). In user interfaces 200A-2200 (FIGS. 2-23) above (and in the underlying logic, including the object model), a cohort may entangle the specimen and shipping together in a manner that possibly results in inefficiencies.

User interfaces 2400-5700 shown in the examples of FIGS. 24-57 along with the underling logic (again, including the object model) may be modified to provide a more clear separation (compared to user interfaces 200A-2200) in the form of a collection aliquot tree, which is a tree data structure. Each node in the tree data structure may be associated with one or more vendor details, where the vendor details can either represent a manipulation of a specimen, shipping content, executing assays (or, in other words, tests), etc. The vendor details may also identify where the assays are to be performed.

In addition, user interfaces 2400-5700 (and the underlying logic) have been modified to promote bulk editing (e.g., with respect to assays) and thereby promote better usability, saving time and potentially processing resources (e.g., processor cycles, memory space, memory bus bandwidth, etc. and associated power consumption). As a result of this bulk editing modification, user interfaces 2400-5700 may remove limitations from user interfaces 200A-2200 (and from the underlying logic) that required SMEs to individually edit every single assay and instead make changes across two or more assays (and possible all of the assays) of a biospecimen (as an example).

User interfaces 2400-5700 (and the underlying logic) may also be modified in comparison to user interfaces 200A-2200 to promote additional flexibility to accommodate the possibly chaotic nature of study design in which vendors, subject groups (e.g., adolescent, adult, etc.), regions, etc. change during the development of a plan for specimen management (which may be referred to as a “specimen management plan”). User interfaces 2400-5700 may provide additional flexibility as described in more detail below to enable more dynamic and flexible changes prior to forming the specimen management plan.

In contrast, user interfaces 200A-2200 may generate the specimen management plan, allow the SME to make changes to the generated specimen management plan, and then regenerate the specimen management plan, but this may result in lost work. Every time the SME regenerates a plan there is a likelihood of lost work because biospecimen definitions are quite dynamic (especially in the early phases of the study design). As such, SMEs may regenerate specimen management plans a number of times increasing the likelihood of lost work and accompanying user frustration as well as consume significant amounts of computing resources.

User interfaces 2400-5700 may, however, overcome this lack of flexibility by configuring the biospecimens with a possible goal of allowing specimen management plans to be deterministically generated such that the specimen management plans are immutable and may always reflect what the SME wants from the specimen management plan. This deterministic generation of specimen management plans in combination with the more dynamic biospecimen updates prior to specimen management plan generation enables user interfaces 2400-5700 (and the underlying logic) to provide more flexibility (compared to user interfaces 200A-2200) in configuring the biospecimen such that a definitive specimen management plan can be generated.

In this respect, system 100 may be modified to shift one or more interactions with what is being collected and which entity is doing the testing before you even bring in the schedule. However, system 100 may provide the shifted interactions, but may also enable groups and proto-plans (uncommitted plans), which may allow for conceptualization and even the visualization of what is happening with each of the biospecimens (a form of sample) earlier in the specimen management plan generation process. Once the SME generates the specimen management plan(s), system 100 may reflect all of the detail (regarding specimen management) upfront. The SME may review the specimen management proto-plans to identify any errors prior to ever committing a proto-plan and, only once committed after the review, generate a formal specimen management plan (which may be submitted to a regulatory body for purposes of gaining approval to pursue a study).

To illustrate, a possibly common issue that arises during specimen management plan generation is that the SME might inadvertently add something inconsistent in the biospecimen profiles, which resulted in multiple aliquots or multiple collections that were not really needed. Again, user interfaces 2400-5700, like user interfaces 200A-2200, may attempt to provide compact collection of samples, such as biospecimens (e.g., blood, tissue, etc.), to potentially alleviate subject burden during sample collection, time wasted collecting samples that require multiple visits, increased shipping of samples and associated costs, etc. These inconsistences may reduce the compactness of the specimen management plans, which the SME will have to fix in order to provide as compact a specimen management plan as possible. By allowing SMEs to verify data prior to specimen management plan generation, not only does system 100 potentially provide more flexibility, but may also enable errors or oversights to be more quickly identified such that the entire specimen management plan may not need to be regenerated (or not regenerated as many times compared to system 100 navigated via user interfaces 200A-2200).

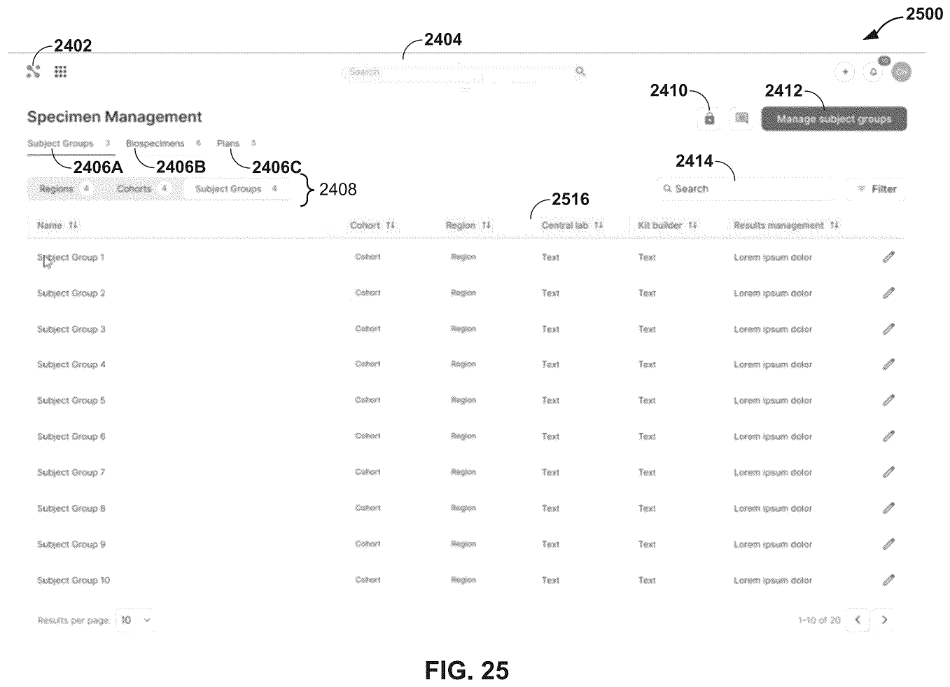

Referring first to the example of FIG. 24, a specimen management user interface (SMUI) 2400 may represent an example of SMUI 131B. SMUI 2400 may include a home icon 2402, a global search bar text input 2404, plan formation tabs 2406A-2406C (“plan formation tabs 2406”), view selectors 2408, a lock icon input 2410, a manage subject group button 2412, and a local search bar text input 2414, and a table 2416. While the description omits some of the various graphical user interface elements shown in FIG. 24, it should be understood that unless otherwise specified these elements have their common and well understood operation (e.g., a pencil icon input may specify an edit operation of the associated, in this instance, entry in table 2416, a comment icon input may allow for a dialogue to be opened with another SME working on specimen management and or any other aspect of the study design, etc.). Moreover, various elements in subsequent FIGS. 25-57 that are described with respect to specimen management user interface 2400 FIG. 24 may not be described again (or denoted with reference numerals) unless some functionality in the flow of FIGS. 25-57 requires further elaboration (at which point a reference numeral may be shown for purposes of readability).

Returning to the example of FIG. 24, home icon 2402 may result in SMUI 2400 returning to some form of home user interface or otherwise navigate between different portions of the study design user interface. Global search bar text input 2404 may enable entry of text that results in a global search of all information within the study design, returning results by which to navigate to a user interface that displays the information requested via the entered text.

Specimen Management navigation tabs 2406 include a subject group tab 2406A, a biospecimens tab 2406B, and a plans tab 2406C. Subject group tab 2406A may represent a tab in which table 2416 includes an entry for each subject group. Biospecimens tab 2406B may represent a tab in which a table (shown below in more detail) includes an entry for each biospecimen. Plans tab 2406 may represent a tab that provides the proto-plans for review (where the proto-plans are associated with Collection Groups that are specified under the scope of the Biospecimens tab) and allows for generation of the specimen management plan. Each of biospecimens tab 2406B and plans tab 2406C are described in more detail below.

In the example of FIG. 24, subject groups tab 2406A has been selected, which results in SMUI 2400 presenting view selectors 2408, local search bar text input 2414, and table 2416. View selectors 2408 may enable selection of regions, cohorts, and/or subject groups, which transition table 2416 to display regions, cohorts, and/or subject groups respectively. Local search bar text input 2414 may enable user interaction through text entry that searches for corresponding text in the entries of the currently shown table 2416 (e.g., regions, cohorts, and/or subject groups).

As noted above, the concept of a subject group is introduced in user interfaces 2400-5700 shown in FIGS. 24-57, replacing the use of cohort in user interfaces 200-2200 shown in FIGS. 2-23 above for a similar concept. Cohorts have been demoted a level, but still reside within user interfaces 2400-5700. A subject group may include a region and a cohort. A region may represent a geographical region or a political region that may require variations in processing. As shown in SMUI 2400, table 2416 shows regions, cohorts, and subject groups (which are a function of regions and cohorts), which enables vendor default selections on a region by region basis (which may alleviate some manual editing activity compared to user interfaces 200A-2200). As such, SMUI 2400 may provide for a form of bulk editing in terms of setting default values that SM microservice 130B may propagate into the specimen management plans when SM microservice generates the specimen management plans. Moreover, the regions may also have another effect of potentially restricting or mandating certain assays to be processed (which is described in more detail below).

SM microservice 130B may automatically generate subject groups based on a cross product of the region and the cohorts (which may represent a set of subgroups). SM microservice 130B may assign each subject group a default name (e.g., region:cohort). By generating default names that potentially have added meaning, SMUI 2400 may promote a better user experience compared to the user experience provided by user interfaces 200A-2200.

Referring next to FIG. 25, an SMUI 2500 is shown that represents another example of SMUI 131B, and is the result of an SME or other user selecting subject groups in view selectors 2408, thereby resulting in one or more of generation and display of a table 2516. SMUI 2500 includes table 2516 with entries identifying a subject group and columns showing the subject group name, the cohort, the region, the central lab, the kit builder (for sample collection), while also allowing each entry to be edited (e.g., using the pencil icon for each entry in table 2516).

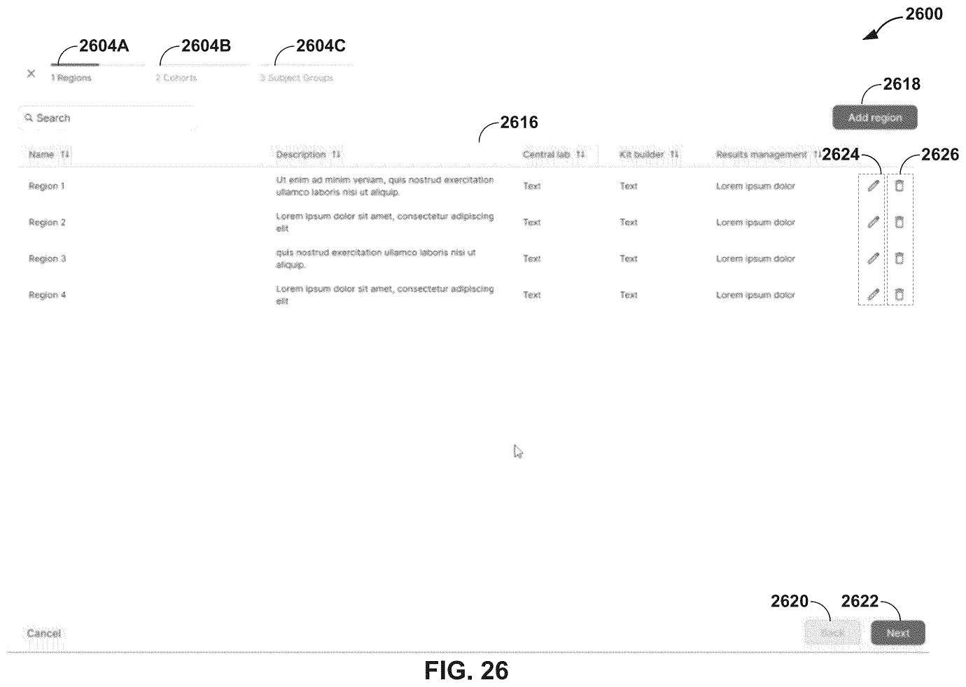

As a result of no longer manually creating subject groups (or, as such subject matter groups were referred when described with respect to FIGS. 2-23, cohorts), SMUI 2500 provides a way to manage the subject groups in a transactional manner (which is why SMUI 2400 and 2500 includes the manage subject groups button 2412). Manage subject groups button 2412 may represent a button to initiate execution of transactional management of subject groups (and thereby may present more flexibility to change or otherwise alter subject group definitions). The SME or other use may next select manage subject groups button 2412 to transition from SMUI 2500 to SMUI 2600 shown in the example of FIG. 26.

In the example of FIG. 26, an SMUI 2600 may represent an example of SMUI 131B. SMUI 2600 may include tabs 2604A-2604C (tabs 2604), a table 2616 (having entries populated based on which of tabs 2604 is/are selected), an add regions button 2618, a back button 2620, and a next button 2622. Tabs 2604 may include a regions tab 2604A, a cohorts tab 2604B, and a subject groups tab 2604C. Regions tab 2604A may represent a tab, that when selected, causes SMUI 2600 to populate table 2616 with regions (if any are defined). Add regions button 2618 may represent a button that exposes a dialogue to add and define a new region to table 2616. Similar buttons may be present for different tabs 2604 (e.g., an add cohorts button to add new cohorts when tab 2604B is selected and/or an add subject group button to add new subject groups when tab 2604C is selected, despite subject matter groups being normally created automatically as the cross product of the region and cohort). Back button 2620 may represent a button that, when selected, causes SMUI 2600 to move backwards through the transactional configuration of subject groups, while next button 2622 may represent a button that, when selected, causes SMUI 2600 to move forwards through the transactional configuration of subject groups.

In operation, the first time subject groups for a study are being designed, the SME or other user traverse SMUI 131B to arrive at SMUI 2600 and begin entering regions and cohorts. SMUI 2600 may, upon receiving the regions and cohorts, generate the subject groups. SMUI 2600 may, for each entry, include an edit icon input 2624 (or, in other words, a pencil icon input 2624) and a delete icon input 2626 (or, in other words, a trashcan icon button 2626). Edit icon input 2624 may represent a button that, when selected, causes SMUI 2600 to expose a dialogue by which editing of the associated entry (in this case, region) in table 2616 may occur. Delete icon input 2626 may represent a button that, when selected, causes SMUI 2600 to expose a dialogue by which deletion of the associated entry in table 2616 may occur. As such, SMUI 2600 may enable addition, editing, and deletion of, in this instance, regions to provide greater flexibility and allow for study specific design to possibly accommodate the needs of that individual study.

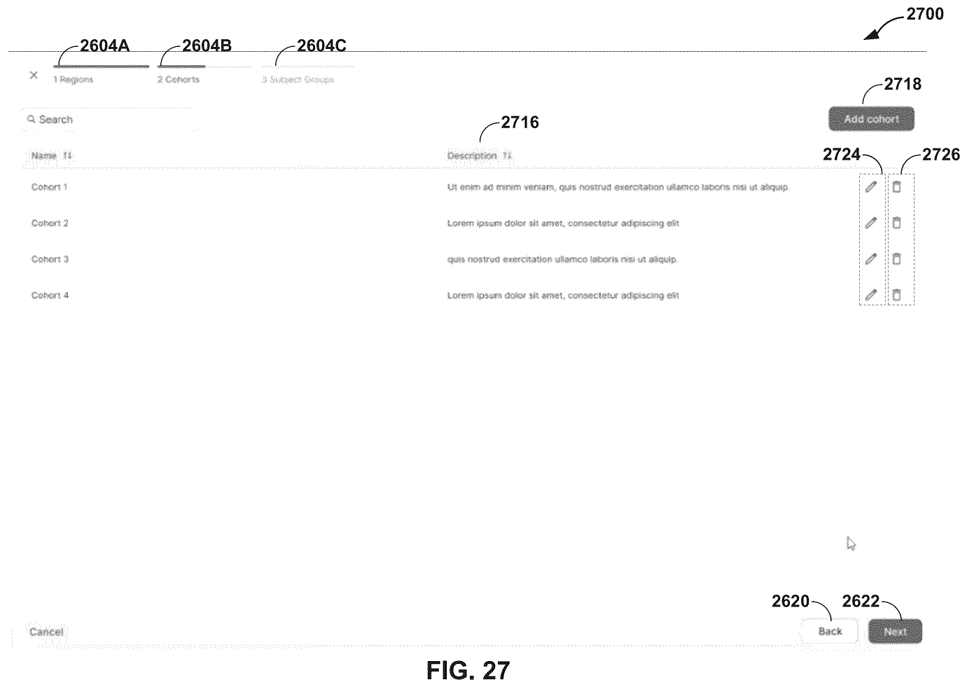

Upon deletion of a region via one or more of detect icon inputs 2626, SMUI 2600 may remove one or more subject groups due to the cross product nature of how subject groups are generated. As a result, work may be lost, and SMUI 2600 may expose a dialogue to warn the user of the lost work. This functionality of SMUI 2600 described with respect to regions also applies to cohorts when selected via cohorts tab 2604. Assuming that SMUI 2600 receives a selection of cohorts tab 2604 (or a selection of next button 2622), SMUI 2600 may transition to SMUI 2700 shown in the example of FIG. 27.

SMUI 2700, as shown in FIG. 27, may represent another example of SMUI 131B, and includes a table 2716 and an add cohorts button 2718. Table 2717 includes one or more entries that each define a cohort by name and description, where each entry includes a respective one of edit icon inputs 2724 and a respective one of delete icon inputs 2726. Edit icon input 2724 may be similar to edit icon inputs 2624, except for cohorts, while delete icon inputs 2726 may be similar to delete icon inputs 2626. In this respect, SMUI 2700 may support the addition of new cohorts (via add cohort button 2718), editing of cohorts (via edit icon input 2724), or deletion of cohorts (via delete icon input 2726) similar to description above with respect to regions.

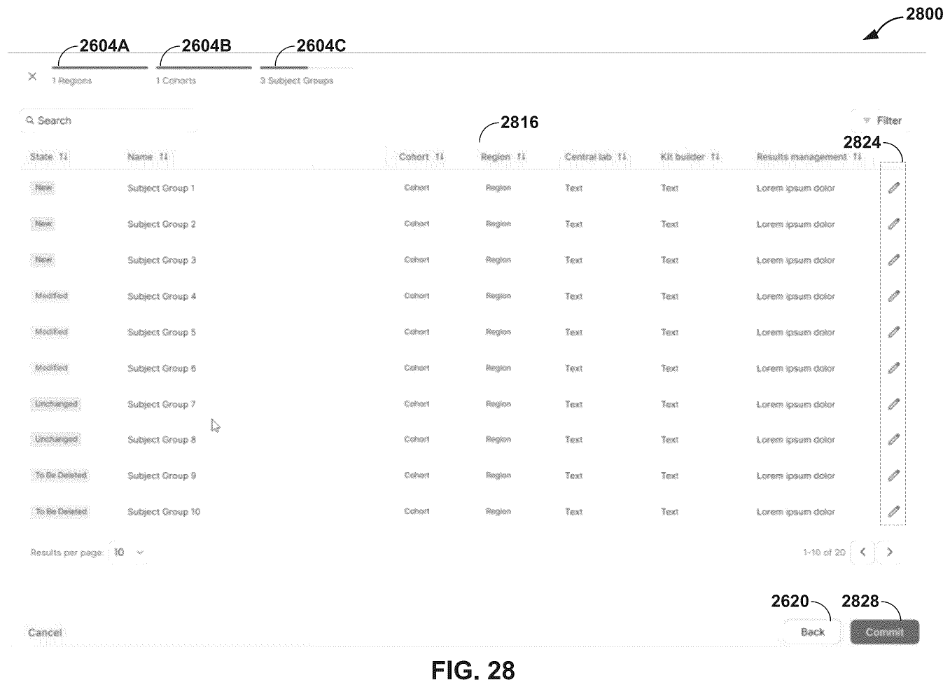

Assuming a selection of subject groups tab 2604C or a selection of next button 2622 (where back button 2620 may, when selected, cause SMUI 2700 to transition back to SMUI 2600), SMUI 2700 may transition to an SMUI 2800 shown in the example of FIG. 28. In FIG. 28, SMUI 2800 may represent another example of SMUI 131B, and includes a table 2816. Table 2816 includes entries, each entry indicates a state, a name, a cohort, a region, a central lab, a kit builder, and results management for each respective subject group along with edit icon inputs 2824 (which functions similarly to edit icon inputs 2624 and/or 2724 that, when selected, may expose a dialogue to edit the corresponding subject group.

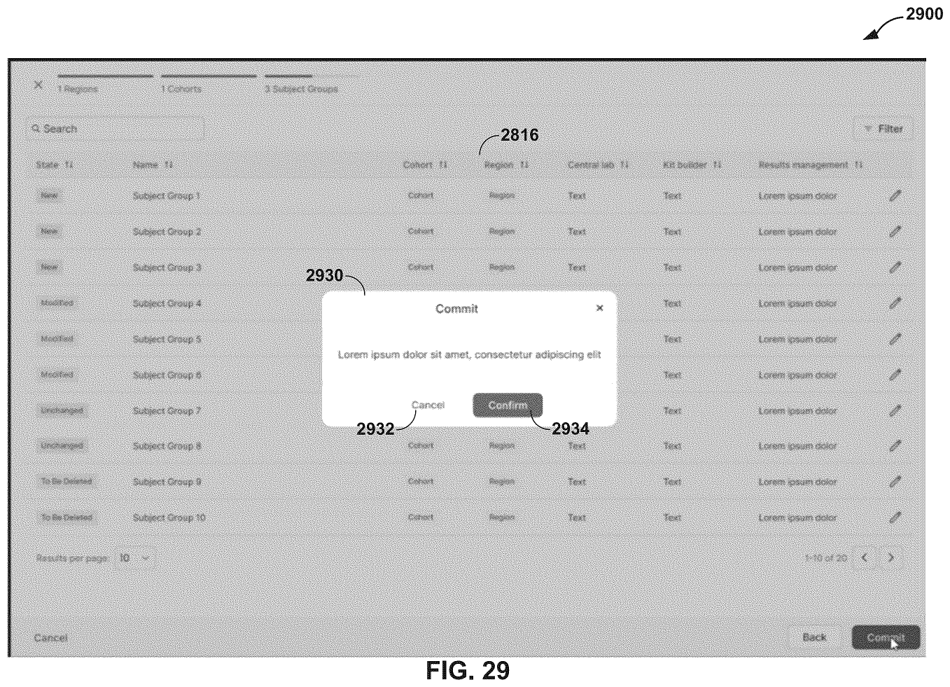

SMUI 2800 may further include a commit button 2828, which represents a button that, when selected, may cause SMUI 2800 to transition to SMUI 2900 shown in the example of FIG. 29. Again, SMUI 2900 may represent an example of SMUI 131B. SMUI 2900 includes a dialogue 2930 as a result of entries in table 2816 having items with a status of “To Be Deleted,” shown as the last two entries in table 2816. Dialogue 2930 may represent an overlay (e.g., such as a pop-up box, dynamic element, or any other sort of dialogue graphical user interface element) that provides a warning to the user that “Subject Group 9” and “Subject Group 10” will be deleted. Dialogue 2930 may include a cancel button 2932 and a confirm button 2934. Cancel button 2932 may represent a button that, when selected, causes SMUI 2900 to cancel the commit functionality, where confirm button 2934 represents a button that, when selected, causes SMUI 2900 to proceed with the deletion of the entries in table 2816 (e.g., named “Subject Group 9” and “Subject Group 10”).

One possible business problem that may be solved through the separating regions from cohorts arises from an operation and regulatory perspective. One possible reason SMUI 2900 separates regions and cohorts and explicitly specifies regions is that sponsor companies (where a sponsor refers to a sponsor of the study) may operate in many different regions and depending on the region the study may use different labs. So the SME may operate at the region level to specify, at the region level, a particular central lab and that then propagates through all of the regions. Again, specifying a central lab per region may represent another form of a bulk edit propagation of the central lab occurs automatically, where previously there may have been difficulties managing the how the flow works for different countries with different contracts with the vendors selected by the sponsor companies.

Separating out cohorts from regions may involve more of a regulatory issue in that various cohorts are limited or otherwise differ in terms of collection and specimen management. For example, for pediatric trials where depending on the weight of the subject, the blood volume limits are different for ethical reasons, health reasons, etc. As such, an adolescent cohort may have limits imposed on collection of blood and/or other biospecimens that would alter the specimen management plan.

In this way, system 100 may combine the operational details for the labs as well as what variations might need to happen and with what samples are being collected. In addition, certain regulatory agencies in different countries may require a certain specimen to be collected or exclude the specimen from being collected. System 100 may provide for the definition and generation of the subject groups in the manner shown and described above with respect to SMUI 2400-2900 to address these operational and regulatory issues.

Given the somewhat chaotic nature of defining specimen management plans, system 100 may address an expectation that the SME would set this up as early as possible because, once the subject groups are generated, system 100 may inject the subject groups into the biospecimens during the biospecimens configuration. System 100 includes SMUI 131B that provides a way to traverse back and forth between SMUIs 2400-2900 because, as one example, the design changes (such as the study expands to a new region, change the cohort model, etc.).

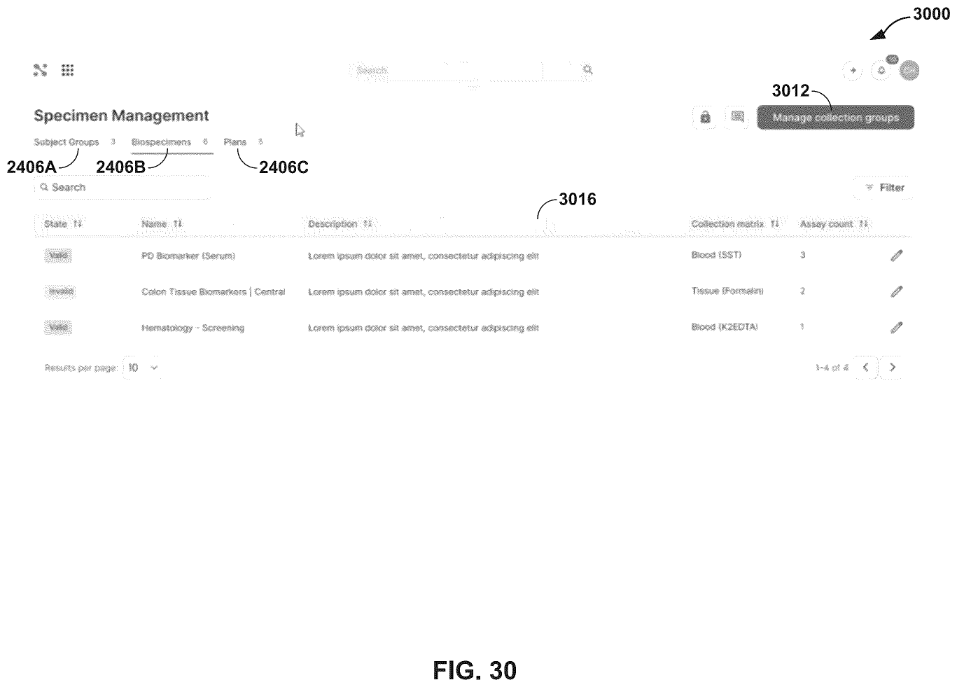

Assuming SMUI 2900 receives a selection of confirm button 2934, SMUI 2900 transition back to a home SMUI 3000 shown in the example of FIG. 30. Home SMUI 3000 may represent another example of SMUI 131B and provides a central hub UI by which to navigate between subject groups, biospecimens and plans. In the example of FIG. 30, SMUI 3000 is similar to SMUI 2400 except SMUI 3000 has biospecimens tab 2406B selected. SMUI 3000 includes a manage collection groups button 3012 and a table 3016. Manage collection groups button 3012 may operate similarly to manage subject groups button 2412, but with respect to collection groups as described in more detail below. Table 3016 may include entries, each entry identifying a respective one of collection groups by state (e.g., valid or invalid), name (e.g., “PD Biomarker (Serum), “Colon Tissue Biomarkers,” and “Hematology—Screening”), description, collection matrix (e.g., “Blood (SST),” “Tissue (Formalin),” and “Blood (K2EDTA)”), and assay count.

As noted above, one possible reason biospecimens are not shown, but rather collection groups, is that system 100 is configured to generate compact collection plans (possibly as much as possible) to potentially minimize the cost and also to minimize patient burden. SMUI 3000 may provide a way, similar to subject groups tab 2406A, to review, edit, delete, etc. automatically generated collection groups, which combines the collection of similar or the same substance in the same collection matrix (e.g., an SST, a formalin container, a K2EDTA, etc.). The SME may need to override what was automatically configured, which again can lead to the loss of work. SMUI 3000 may provide a way (through further interactions described in more detail below) to control the combination of biospecimens in the preparatory phase before generating the plan through the collection groups, and by doing so ensuring that those generated plans are complete and accurate and therefore removing the need for plan editing subsequent to generation.

Collection groups may establish a grouping of compatible biospecimens, where again compatible refers to having the same collection matrix and medium. In other words, compatible may mean that the substance that is collected and the medium into which the substance is collected are the same (e.g., blood into an SST, blood into a K2EDTA, or tissue into a formalin container). System 100 may determine whether the standards grouping for the particular biospecimen is compatible and then combine the compatible biospecimen collections into a collection group. For example, a standards grouping compatible is that all safety labs for blood serum collection could be combined. However, biomarker combinations could be specified to disallow combination. This standards grouping example may be defined as a rule that the sponsor company (“the sponsor”) configures in the standards such that, when all the specimens have been specified for the study, system 100 may produce collection groups.

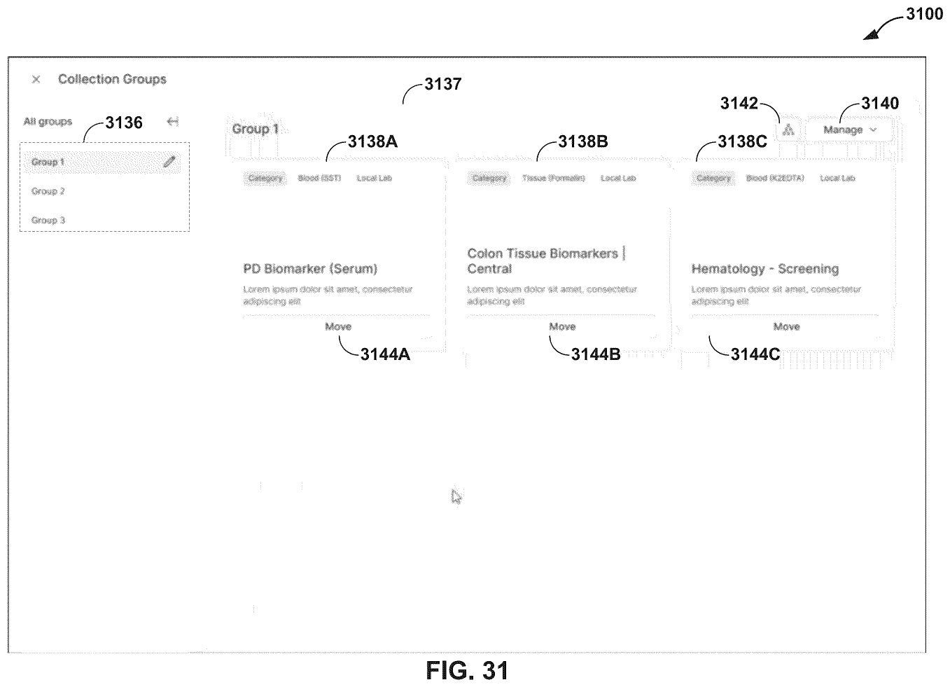

Assuming SMUI 3000 receives an indication (or, in other words, selection) that manage collections group button 3012 has been selected, SMUI 3000 may transition to SMUI 3100 shown in the example of FIG. 31. SMUI 3100 may represent yet another example of SMUI 131B and may include, as shown in the example of FIG. 31, group selector inputs 3136, biospecimen cards 3138A-3138B, a hierarchy icon input 3242, a manage dropdown box 3140.

Each of group selector inputs 3136 may represent a button that, when selected, may cause SMUI 3100 to switch between Groups 1-3, which would update pane 3137 to present biospecimen cards associated with the selected one of Groups 1-3. In this example, Group 1 is shown as selected, and pane 3137 displays biospecimen cards 3138A-3138C (“biospecimen cards 3138”). Each of biospecimen cards 3138 may specify at a high level a company name, a biospecimen type along with the collection matrix (e.g., “Blood (SST)”), and a collection site (e.g., “local lab”), a name “PD Biomarker (Serum)”), and a description. Each of biospecimen cards 3138 may also include a respective one of move buttons 3144A-3144C (“move buttons 3144”). Each one of move buttons 3144 may represent a button that, when selected, may expose a dialogue by which to move the corresponding biospecimen from Group 1 to a different one of the Groups (e.g., Group 2, Group 3, or possibly a new Group).

Hierarchy icon input 3142 may represent a button that, when selected, transitions SMUI 3100 to a different SMUI view in which a selected collection group is presented as a hierarchy of nodes reflecting the collection and transfer of a biospecimen in the group. Manage dropdown box 3140 may represent a dropdown box (although the dropdown box can be replaced with other graphical elements, such as a radio box list, a checkbox list, etc.) by which to manage a selected one or more (or possibly all) of biospecimen cards 3138.

With respect to collection groups, system 100 may not name collection groups generically (similar to the subject groups). Instead, system 100 may reference the standard to determine the name (where the standard can use any feasibly understandable name). System 100 may access the standard and retrieve the name and automatically populate the names here (e.g., in group selection 3136). Using the standard may allow system 100 to accommodate incremental additions of biospecimens to the study, which system 100 may automatically group (possibly as long as the group name has not changed via edit). If the name of the collection group has been changed, the SME may have to manually move biospecimens between groups (or create a new group) using the move buttons 3144.

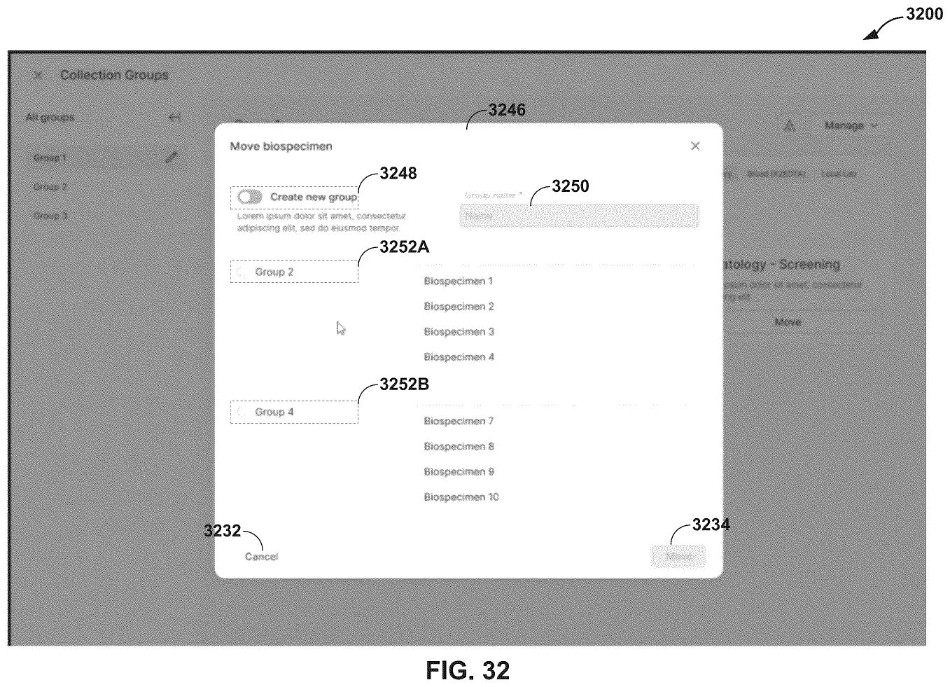

Assuming SMUI 3100 receives a selection of move button 3144A, SMUI 3100 transitions to SMUI 3200 as shown in the example of FIG. 32. SMUI 3200 is another example of SMUI 131B, which includes a dialogue 3246. Dialogue 3246 may represent an overlay or other graphical element by which to facilitate movement of a biospecimen to another different group (including a new group).

Dialogue 3246 may include a create new group toggle 3248, a group name text entry box 3250, group selectors 3252A and 3252B (“group selectors 3252”), a cancel button 3232, and a move confirmation button 3234. Create new group toggle 3248 may represent a toggle or other graphical element by why to create a new group, while group name text entry box 3250 represents a text box by which to specify the new group name. Group selectors 3252 may represent a radio button or other graphical element that, when selected, indicates to which of the compatible groups Group 2 or Group 4 that Group 1 should be moved. Cancel button 3232 may represent a button that, when selected, may cancel the move of Group 1, while move confirmation button 3234 may represent a button that, when selected, may confirm the move.

In the example of FIG. 32 it is assumed that SMUI 3200 received an indication that create new group toggle 3248 was selected, and in response to the selection of create new group toggle 3248, SMUI 3200 may allow for entry of a group name to group name text box 3250 (or may expose a different dialogue). Assuming that this new group was created, SMUI 3200 may add another group card to group cards 3138, and the newly added group would be visible in dialogue 3246 (e.g., as Group 4). SMUI 3100 shown in FIG. 31 may present this new Group in group selection input 3136.

System 100 may, in this manner, allow for biospecimens to be moved between existing and new groups. System 100 may support iterative interactions to view and move biospecimens between groups (so long as supported by the standard). Biospecimen collection may, in this way, be tailored to a particular study or to accommodate new groups for various reasons.

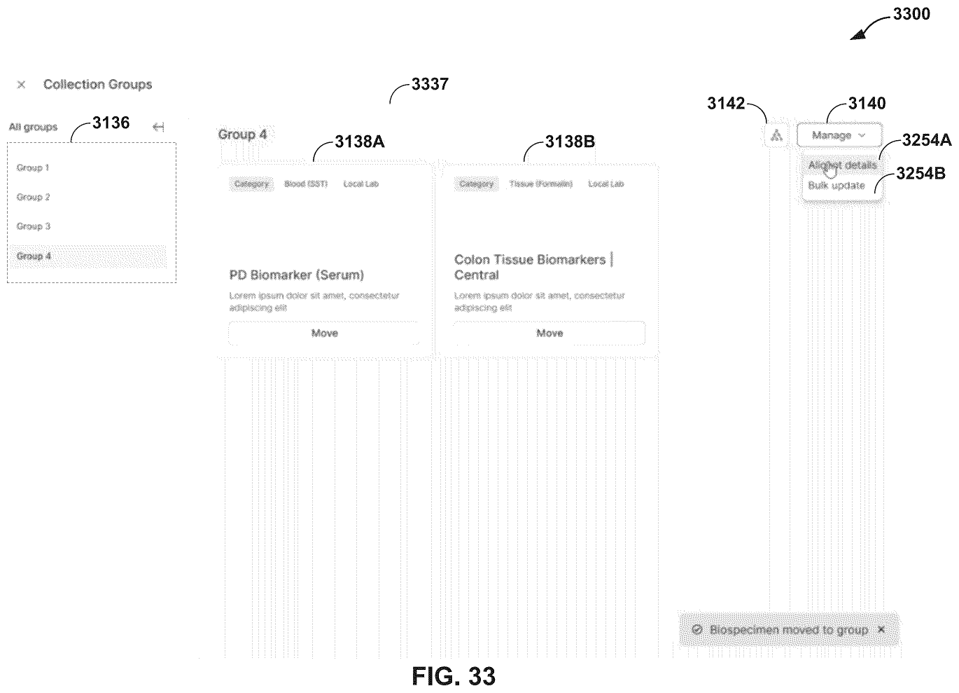

Assuming that biospecimen cards 3138A and 3138B are moved to Group 4 through this iterative process (e.g., selecting move buttons 3144A and 3144B iteratively and traversing dialog 3248, selecting group selector 3252B and then move confirmation button 3234), SMUI 3200 may move biospecimen cards 3138A and 3138B to Group 4, and transition to SMUI 3300 shown in the example of FIG. 33. SMUI 3300 may represent another example of SMUI 131B, and includes group selector 3136 (with the addition of Group 4) and a pane 3337. Pane 3337 may include biospecimen cards 3138A and 3138B that were moved from Group 1 to Group 4, confirming that biospecimen cards 3138A and 3138B were moved.

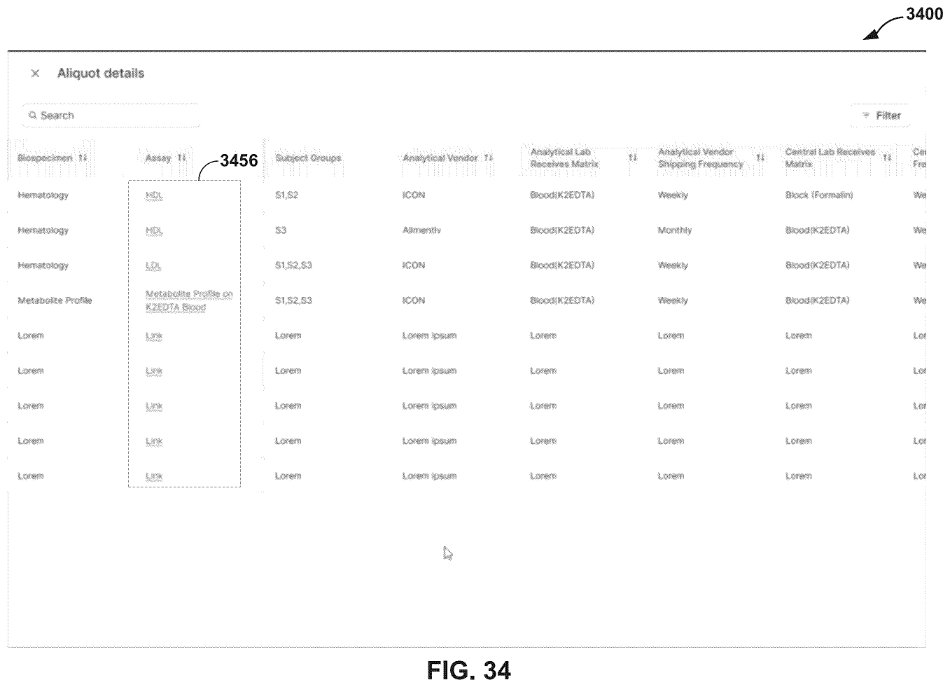

In addition, it is assumed that SMUI 3300 received an indication that manage dropdown box 3140 was selected thereby causing SMUI 3300 to expose a dropdown list including aliquot details selector 3254A and bulk update selector 3254B (along with other potential selectors, such as a Kit Builder selector, an Override vendor selector, etc., which are not shown for ease of illustration purposes). Responsive to receiving an indication that aliquot details selector 3254A was selected, SMUI 3400 may transition to SMUI 3400 shown in the example of FIG. 34. SMUI 3400 may represent a further example of SMUI 131B and presents a view of proto-plans, which may refer to a miniature plan (or a plan subset) that includes only biospecimens that are part of the collection group. SMUI 3400 may produce the proto-plan to also include an expected output per biospecimen assay combination.

Such proto-plans may facilitate review by the SME to identify any inconsistencies. That is, the values used for the assay operational details may include the biospecimens of that group, and the SME may want these to be as consistent as possible because variances could result in additional collections and aliquots. SMUI 3400 may provide a way by which to quickly inspect the consistency of the values, and further determine whether any inconsistences were deliberate or unintentional (or, in other words, oversights).

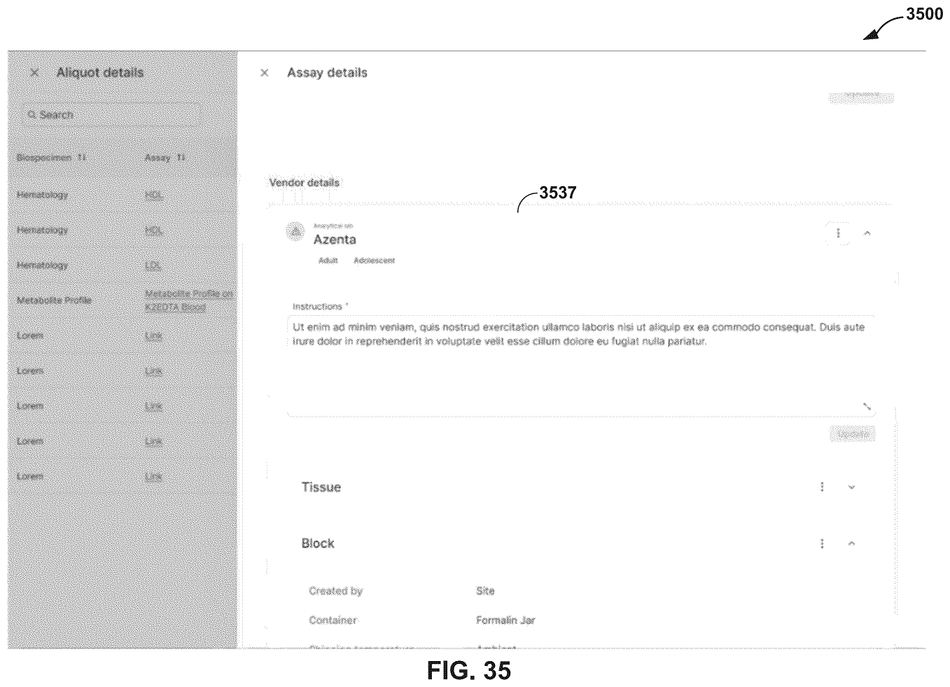

In the event further review or corrections are required to correct for unintentional inconsistencies, SMUI 3400 may include assay links 3546. Assay links 3546 each represent a respective link to an assay within system 100. Assuming SMUI 3400 receives an indication that one of assay links 3456 was selected (e.g., the top most “HDL” link in links 3456), SMUI 3400 transitions to SMUI 3500 shown in the example of FIG. 35.

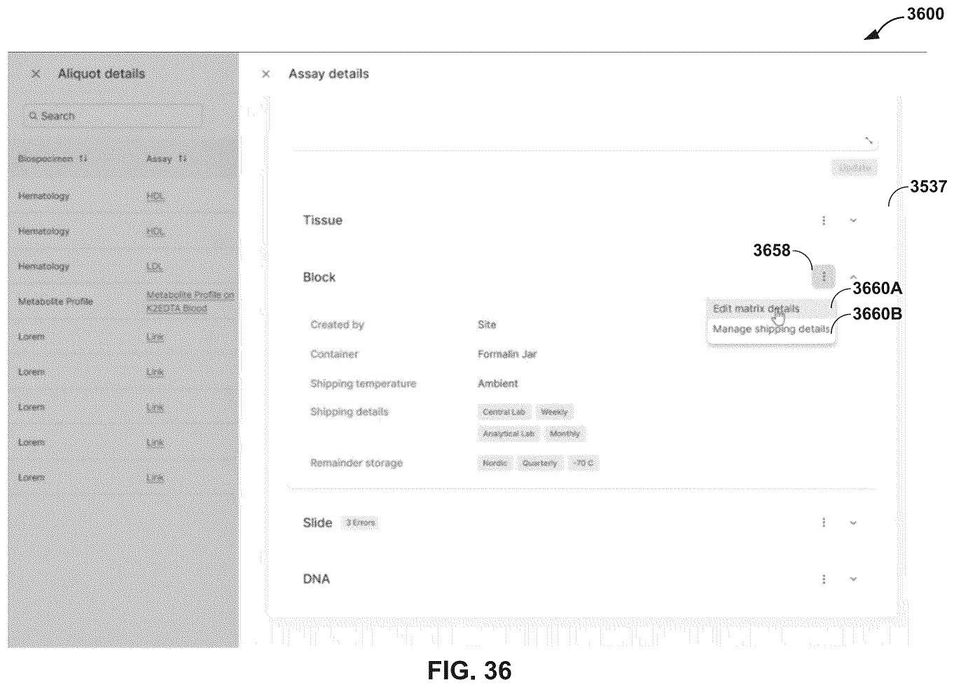

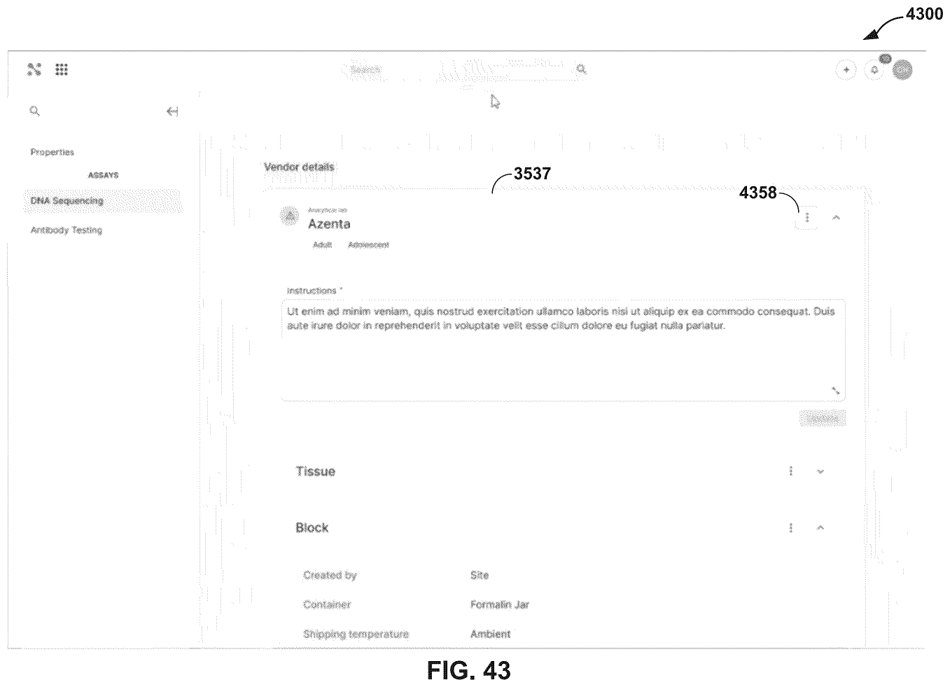

In the example of FIG. 35, SMUI 3500 includes vendor details pane 3537, which represent a graphical element for denoting where vendor details are specified, including various aliquot and assay details (where SMUI 3500 may represent a shortcut to improve usability for the SME when discovering an issue with the proto-plan, but the SME may also perform biospecimen config work via SME 4300 of FIG. 43 described in more detail below). SMUI 3500 may transition to SMUI 3600 shown in the example of FIG. 36 responsive to an indication that the vendor details pane 3537 has been scrolled down.

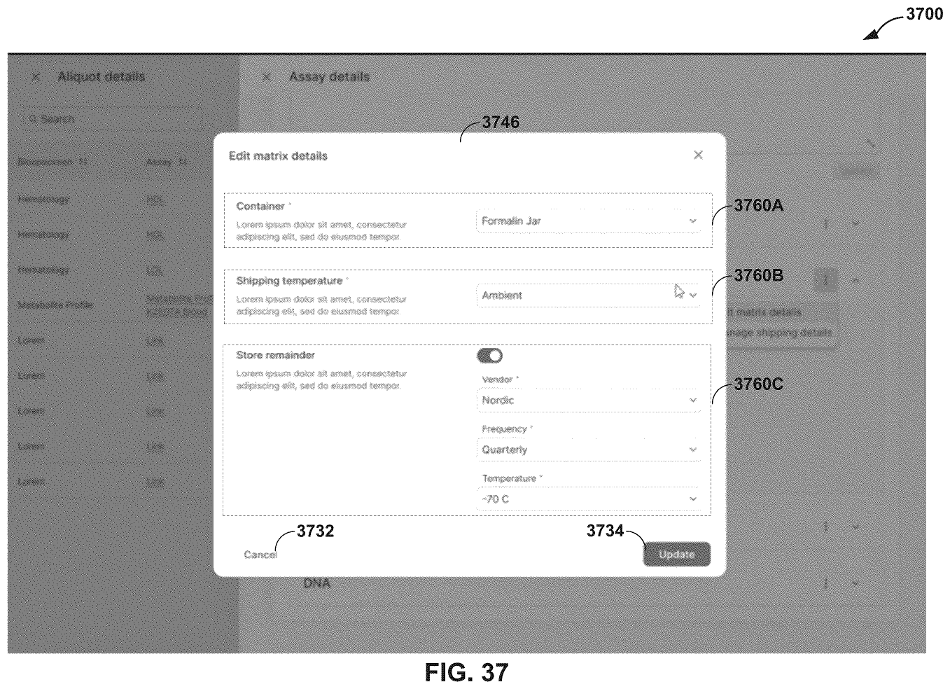

Both SMUIs 3500 and 3600 represent a user interface that enables changes to aliquot details. Referring to the example of FIG. 36, SMUI 3600 includes vendor details pane 3657 (although scrolled down to expose additional assay details). Vendor details pane 3657 includes an additional action icon input 3658 (shown as a three vertical dot icon 3658). Responsive to receiving an indication that additional action icon input 3658 has been selected, SMUI 3600 exposes an edit matrix details action 3660A and a manage shipping details action 3660B. Edit matrix details action 3660A may represent a link to an edit matrix details SMUI 3700 whereupon SMUI 3600 may, responsive to an indication that edit matrix details action 3660A was selected, transition to SMUI 3700 shown in the example of FIG. 37.

As illustrated in the example of FIG. 37, SMUI 3700 includes dialogue 3746, which may represent a graphical element for specifying edits to matrix details (where matrix refers to the collection matrix or, in other words, the collection medium). Dialogue 3746 may include a container section 3760A, a shipping temperature section 3760B, and a store remainder section 3760C. Container section 3760A may enable modification of the type of collection container used to collect the biospecimen. Shipping temperature section 3760B may enable modification of a temperature at which the biospecimen needs to be shipped (if shipped between labs, for storage, etc.). Store remainder section 3760C may include a toggle to activate, whereby dropdowns can be configured to define a vendor, a frequency of storage, and a temperature of storage.

Dialogue 3746 also includes a cancel button 3732 and an update confirmation button 3734. Cancel button 3732 may represent a button that, when selected, causes SMUI 3700 to cancel any edits to the collection matrix. Update modification button 3734 may represent a button that, when selected, causes SMUI 3700 to perform the modifications to the collection matrix and return to the assay details SMUI 3600. SMUI 3600 may receive an indication that the aliquot details SMUI 3500/3600 is to be closed (e.g., by selecting the ‘X’ in the top left corner of SMUI 3600), whereupon SMUI 3600 transitions back to SMUI 3400 shown in the example of FIG. 34. SMUI 3400 may receive an indication that SMUI 3400 is to be closed (e.g., by selecting the ‘X’ in the top left corner of SMUI 3400), which transitions SMUI 3400 back to SMUI 3300.

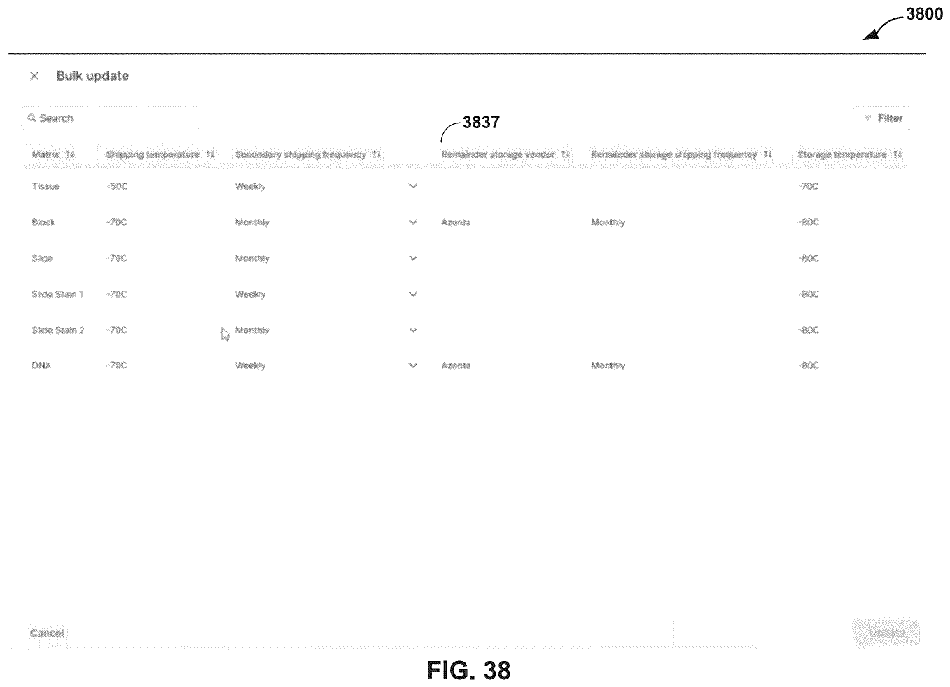

As shown in the example of FIG. 33, SMUI 3300 may include a bulk update selector 3254B. SMUI 3300 may, responsive to receiving an indication that bulk update selector 3254B has been selected, transition to SMUI 3800 shown in the example of FIG. 38. In this example, SMUI 3800 includes a collection matrix details table 3837 that applies across all biospecimen collections, where such biospecimen matrix details may include a matrix name, a shipping temperature for the corresponding matrix, a secondary shipping frequency for the corresponding matrix, a remainder storage vendor for the corresponding matrix, a remainder storage shipping frequency for the corresponding matrix, and a storage temperature for the corresponding matrix. Each of the fields in each entry to matrix details table 3837 may be editable via any conceivable graphical user interface element, including drop boxes, text entry boxes, radio buttons, checkboxes, etc.

As an example, consider that the frequency is incorrectly configured for the entry with a matrix name of block as the current monthly frequency does not align well with the majority of weekly frequencies in other entries to matrix details table 3837. However, because, in the case of tissue, the process goes from raw tissue to block, block to unstained slide, an unstained slide to a stained slide, stained slide to DNA. Each entry presents the processing characteristics and where they are identical within the proto-plan and where they are not identical this will be empty. In any event, SMUI 3800 may receive an indication that the Tissue entry should have a secondary shipping frequency identical to the block secondary shipping frequency (i.e., monthly in this example).

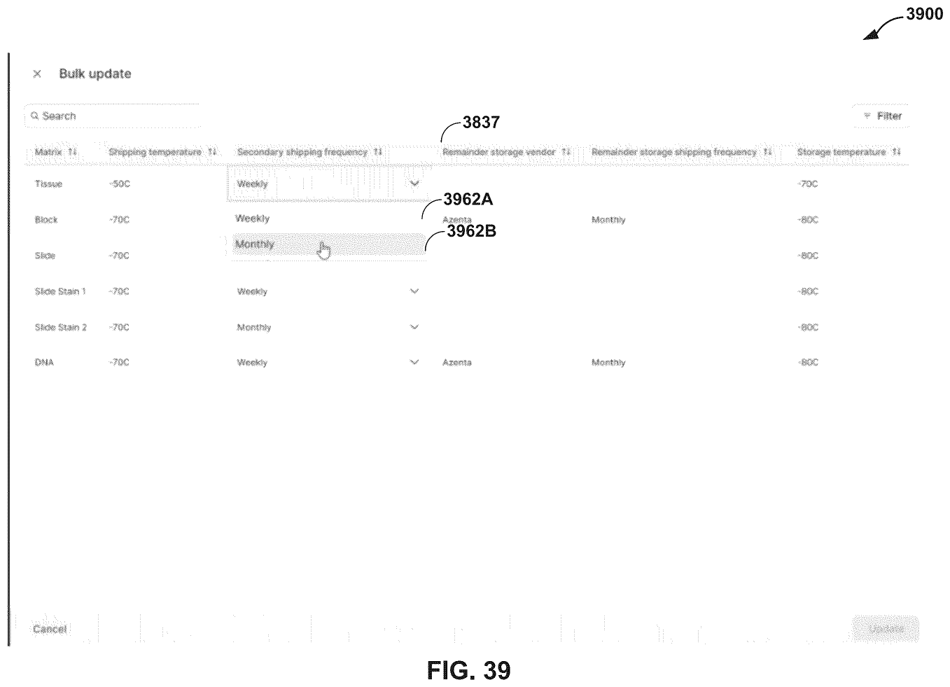

Responsive to receiving an indication that “Weekly” under secondary shipping frequency in the Tissue entry was selected, SMUI 3800 may transition to SMUI 3900 shown in the example of FIG. 39. SMUI 3900 may represent an additional example of SMUI 131B, and may expose two options for the secondary shipping frequency. The first option may include a weekly frequency option 3962A that sets the second shipping frequency to weekly for an aliquoted block, while the second option may include a monthly frequency option 3962B that sets the second shipping frequency to monthly for an aliquoted block. Each of the cells in bulk update table 3837 may be editable, as noted above (including empty cells). This bulk update applies to a particular biospecimen (in this example, Tissue), but each biospecimen may undergo a similar bulk update although the matrix details may change depending on the type of biospecimen.

In this way, SMUI 3900 may allow the SME to get the consistency that is needed to produce the plan the SME desires to meet the study goals. This type of bulk editing may improve the user experience as it can be tedious to manually change individual biospecimen details, such as the matrix details. Moreover, SMUI 3900 may provide a single user interface by which to edit such biospecimen collection details instead of going to a large number of different assays associated with the biospecimen and making the same edits over and over again, a tedious and error-prone set of actions.

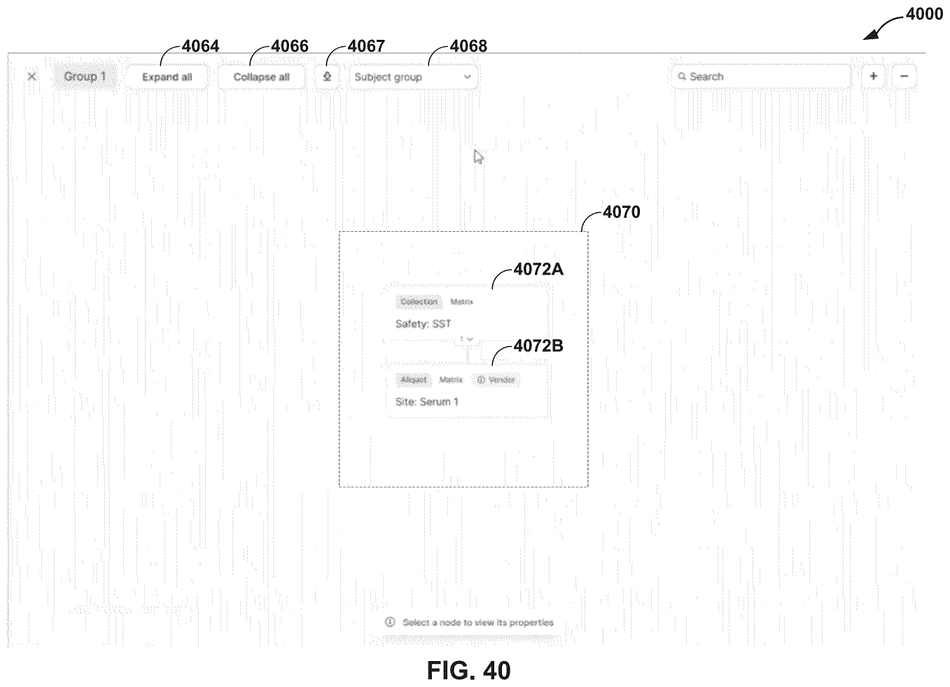

Assuming the user navigates back to collection group SMUI 3100 (FIG. 1) and responsive to receiving an indication that hierarchy icon input 3142 was selected (and that biospecimen card 3138A was selected previously), SMUI 3100 may transition to SMUI 4000 shown in the example of FIG. 40. SMUI 4000 is a further example of SMUI 131B and includes an expand all button 4046, a collapse all button 4046, a download icon input 4067, a subject group dropdown input 4078, and an interactive hierarchical tree view 4070 comprising two interconnected nodes 4072A and 4072B (“nodes 4072”). This tree view provides an alternative means of reviewing the proto-plan that has been configured for the collection group.

Expand all button 4046 may represent a button that, when selected, may cause SMUI 4000 to expand any portion of a collapsed sub-tree (or the entire tree) of interactive hierarchical tree view 4070. That is, interactive hierarchical tree view 4070 may include one or more nodes 4072 that form a sub-tree, and any sub-tree or the entire tree itself may be collapsed to potentially promote more efficient review of the eventual plan (as each tree represents a portion of specimen collection management plan). Expand all button 4046 may expand all collapsed sub-trees and trees to return to a full view of the tree. Collapse all button 4046 may represent a button that, when selected, may cause SMUI 4000 to collapse any expanded sub-trees or the entire tree itself to, as noted above, facilitate review of specific portions of the eventual plan by removing unnecessary views that have already been reviewed.

Download icon input 4067 may represent a button that, when selected, may cause SMUI 4000 to download or otherwise export an image or other format of interactive hierarchical tree view 4070 for, as one example, including in physical or electronic material submitted to regulatory bodies for review and approval of the specimen collection management plan.

Interactive hierarchical tree view 4070 may, as noted above, include nodes 4072, where each of nodes 4072 represents a different collection and/or aliquoting of the underlying biospecimen (which in this example is Blood). In the example of FIG. 40, node 4072A includes a type of collection, and identifies the matrix as “Safety: SST,” where node 4074B depends from node 4072A (as denoted by “1 v” with 1 meaning the number of dependencies and the down arrow indicating depending from) and includes a type of aliquot (performed with respect to the blood collected via node 4072A), a matrix type of “Site:Serum 1,” and a vendor. Each of nodes 4072 may be interactive to allow for further review of vendors and assays performed by those vendors.

In the example shown in FIG. 40, node 4074B is interactive where vendor is selectable (as shown by the circle enclosing an ‘i’ to denote more information) to allow review of vendor details and the assays performed by the vendor. In this respect, interactive hierarchical view tree 4070 represents a collection and aliquot tree, where vendor details are separate from the collection and aliquot tree but still potentially flexibly and conveniently linked.

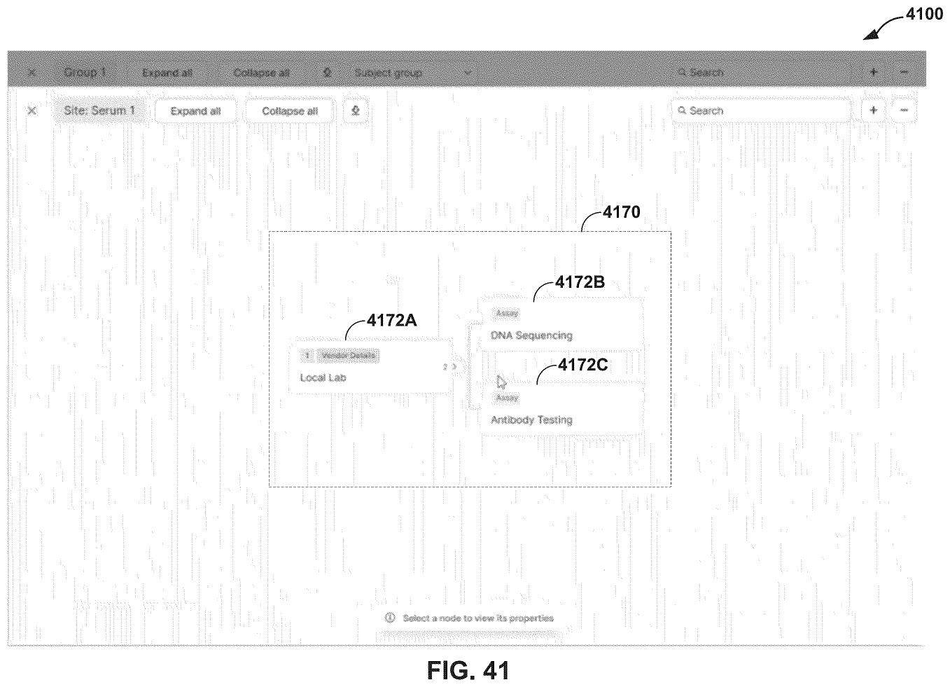

Assuming SMUI 4000 receives an indication that “Vendor” has been selected, SMUI 4000 transitions to SMUI 4100 shown in the example of FIG. 41. SMUI 4100 represents a vendor hierarchical view tree 4170 comprising interconnected nodes 4172A-4172C. Root node 4172A represents vendor details for a local lab and includes two dependencies (denoted by “2>”) to nodes 4172B and 4172C. Node 4172B represents an assay to perform DNA sequencing. Node 4172C represents another assay involving antibody testing. Again, one or more nodes 4172 may be interactive, and selecting any one of nodes 4172 may expose dialogues, overlays, new transitions to additional or previous SMUIs, etc. that provide more details regarding the selected one of nodes 4172.

In other words, vendor interactive hierarchical tree view 4170 may represent that a local lab is performing the processing of the Serum by performing two assays: 1) DNA sequencing; and 2) antibody testing. Vendor interactive hierarchical tree view 4170, while only two levels in the example shown in FIG. 40, may for other biospecimens have even more levels. For example, tissue may have 3 or 4 different levels, as tissue may go through multiple vendors on the specimen journey until being tested or stored. The SME can review views 4070 and 4071 and possibly better understand how that collected specimen is being aliquoted into different transitionary or testing matrixes and how vendors are handling each specimen along the way. This may be beneficial when specifying criteria for central lab handling and confirming that the correct analytical lab vendors are getting the correct assays from the correct sources. The combination of FIGS. 40 and 41 illustrate how collection and aliquot processing are clearly differentiated from vendor handling activities (i.e., shipment and assay processing), thus improving the SME's understandability of the proto-plan and ultimately of the plans generated from the configured biospecimens for the study.

This further illustrates how system 100 may maintain the concept of the physical samples and how the physical samples (where “physical samples,” “samples,” “specimens,” and “biospecimens” are used interchangeably herein) are transformed. System 100 may also, by way of SMUI 4000/4100, highlight all the operational details while managing each separately but also bringing each of them together to provide a comprehensive view of the plan to be generated. This flexibility may offer advantages over using spreadsheets and manually drawing such views while also possibly promoting a better user experience and increasing the speed with which study designs can be produced.

In this way, system 100 may present SMUI 131B in a way that accommodates the iterative approach to specimen management, where an SME may work on a specific biospecimen, move one or more biospecimens around, invoke a tree view, make changes, etc. SMUI 131B may provide an iterative and real time working space for adjusting from the standards for what is required for the study to produce the correct result for the collection and aliquoting.

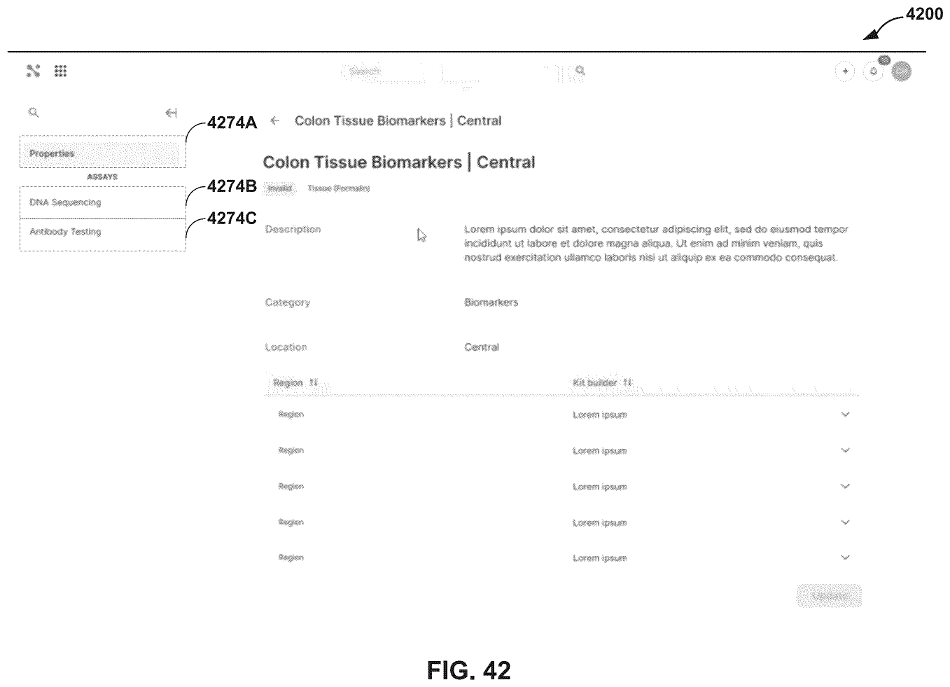

To illustrate consider that SMU 131B returns to home SMUI 3000 shown in FIG. 30, and SMUI 3000 receives an indication that an edit icon input was selected for “Colon Tissue Biomarkers|Central,” SMUI 3000 may, responsive to this indication that the edit icon input was selected for “Colon Tissue Biomarkers|Central,” transition to SMUI 4200 shown in the example of FIG. 42. SMUI 4200 may represent one more example of SMUI 131B, and includes horizontal tabs 4174A-4174C (with horizontal tab 4174A directed to biospecimen properties, horizontal tab 4174B directed to assays involving DNA sequencing, and horizontal tab 4174C directed to assays involving antibody testing).

In SMUI 4200, it is assumed that properties tab 4174A has been selected. It is further assumed that SMUI 4200 receives an indication that DNA sequencing horizontal tab 4274B is selected, and transitions, responsive to the indication that tab 4274B has been selected, to SMUI 4300 shown in the example of FIG. 43.

SMUI 4300 may represent an example of SMUI 131B and selection of horizontal tab 4274B may result in SMUI 4200 jumping to the vendor details for DNA sequencing assay (which may reside below the biospecimen properties within the same UI). SMUI 4300 may be similar to SMUI 3500 shown in the example of FIG. 35. In the example of FIG. 4200, vendor details are assay specific and tied to subject groups. The chips denoted “Adult” and “Adolescent” may represent subject groups. Each vendor details for an assay may always include at least one vendor details, but in some instances, SMUI 4200 may generate two or more vendor details. SMUI 4200 may generate multiple vendor details based on how the subject groups are set up or, in other words, configured. By default, SMUI 4200 may combine compatible subject groups as much as possible, but if there are two sets for compatible subject groups you will end up with two vendor details by default.

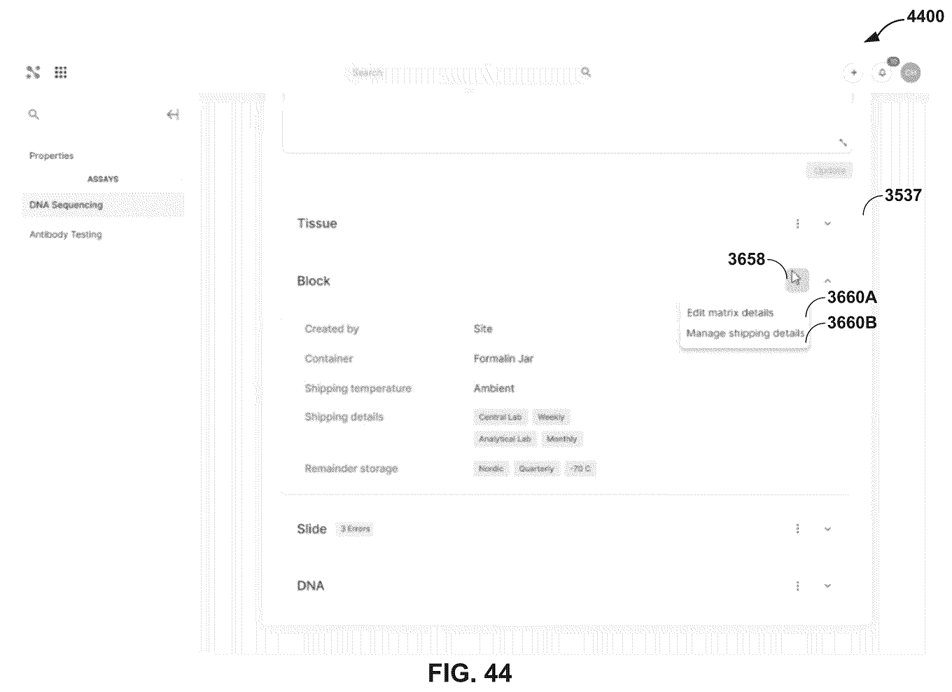

In any event, SMUI 4300 may receive an indication to scroll down further in which case SMUI 4300 transitions, responsive to the scroll down indication, to SMUI 4400 shown in the example of FIG. 44.

SMUI 4400 may also represent an example of SMUI 131B and includes an aliquot pane 4437 directed to a block processing step for tissue. SMUI 4400 may be similar to SMUI 3600 shown in the example of FIG. 36. SMUI 4400 may present the full stack (or, in other words, chain) of matrix types from what is collected (which in this instance is tissue) all the way down to what is tested (which is DNA) and SMUI 4400 may enable editing for each step along the chain (e.g., Tissue is editable, Block is editable, Slide is editable, DNA is editable, etc.). For Block, SMUI 4400 may expose edit matrix details action 3660A and manage shipping details action 3660B responsive to receiving an indication that additional action icon input 3658 was selected. Selection of edit matrix details action 3660A was previously described above.

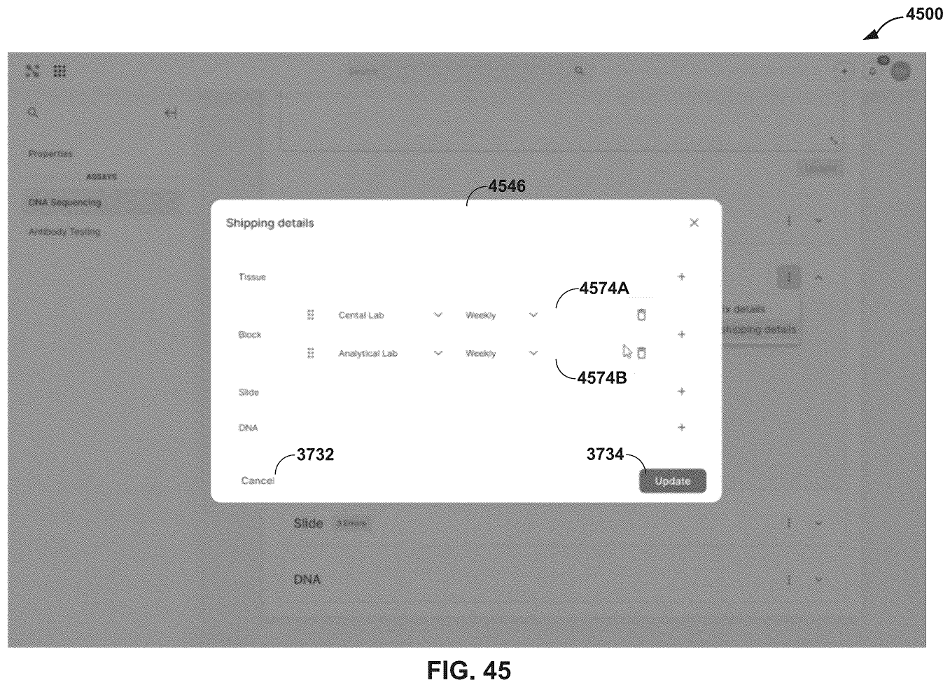

However, responsive to receiving an indication that manage shipping detail action 3660B was selected, SMUI 4400 may transition to SMUI 4500 shown in the example of FIG. 45. Managing shipping details to produce a cost effective and subject burden reducing plan may be a pain point for existing SMEs performing study design. With respect to SMUIs 200A-2200, it may be difficult to definitively generate some of the valid paths by which a sample could pass through.

SMUI 4500 may represent another example of SMU 131B, and may, as shown in the example of FIG. 45, include a shipping details dialogue 4546. Shipping details dialogue 4546 may include, for the Block shipping details, shipping details element 4574A and 4574B (“shipping details elements 4574”). Shipping details element 4574A may represent an element by which to configure shipping to the central lab of the tissue sample, including the lab selection and a shipping frequency. Responsive to an indication that move icon input (represented as two sets of three vertical dots) was selected, SMUI 4500 may enable dragging of one of shipping details elements 4574 to reassign (or, in other words, move or rearrange) the order of shipping (as time flows from top to bottom, and order refers to a time-based ordering). Each of shipping details elements 4574 may also be deleted as evidenced by the delete icon input (shown as a trashcan icon). Cancel button 3732 and update button 3734, which are included in shipping details dialogue 4546 operate as described above with respect to SMUI 3700 (FIG. 37), but with respect to shipping details dialogue 4546.

In other words, shipping details dialogue 4546 may represent a dialogue that indicates to which of the one or more labs a sample in its various forms get shipped until ultimately the sample reaches a lab at which the sample is tested or stored. In the example of FIG. 45, the collection site has collected the sample (tissue) and produced the block (which is why there is no lab specified in “Tissue”). The collection site may perform the initial processing of tissue into a block, and ships the block to the central lab denoted by shipping details element 4574A. The central lab may next pass the block along to the analytical lab denoted by shipping details element 4574B. Both shipping to the central lab and the analytical lab occur weekly. The analytical lab may perform the slide processing, extract the DNA from the slide, and perform the testing.

However, the above example represents just one possible path. In some instances, the SME may want to ship wet tissue to the central lab and the central lab then produces the block, shipping the block to the analytical lab. The analytical lab may next perform some portion of the additional processing and testing, before shipping some aspect of the block (e.g., a slide) to another analytical lab.

Moreover, there are other examples in which the SME may want to modify the shipping details. Consider that the SME may want to have the analytical lab prepare the slides. In this example, the SME would move analytical lab denoted by shipping detail element 4574B (using the two sets of three vertical dots) to the Slides region of dialogue 4546, where the central lab because a pass through of the block. The SME may, in another example, want to change the shipping frequencies from the baseline.

That is, shipping details dialogue 4546 may enable the SME to move labs around in the processing chain of a sample. Shipping details dialogue 4546 may, however, also enable adding new labs (via the add lab icon input shown as a ‘+’ icon), and delete labs as discussed above. The SME may therefore have control over individual legs of the collection aliquot tree, potentially further extending the flexibility and usability of SMUI 131B to account for a variety of circumstances. As an example, the SME may want to split out the slides at the central lab and send them to two different analytical labs (for verification of results, ensuring timely study completion—e.g., in the event slides are lost during shipment, etc.). Shipping details dialogue 4546 may, in other words, represent an aliquot even if that aliquot never leaves the site or never leaves the central lab, where shipping details dialogue 4546 may present the full stack of aliquots and allows configuration of where each aliquot was created, each aliquot was derived, and where each aliquot is shipped.

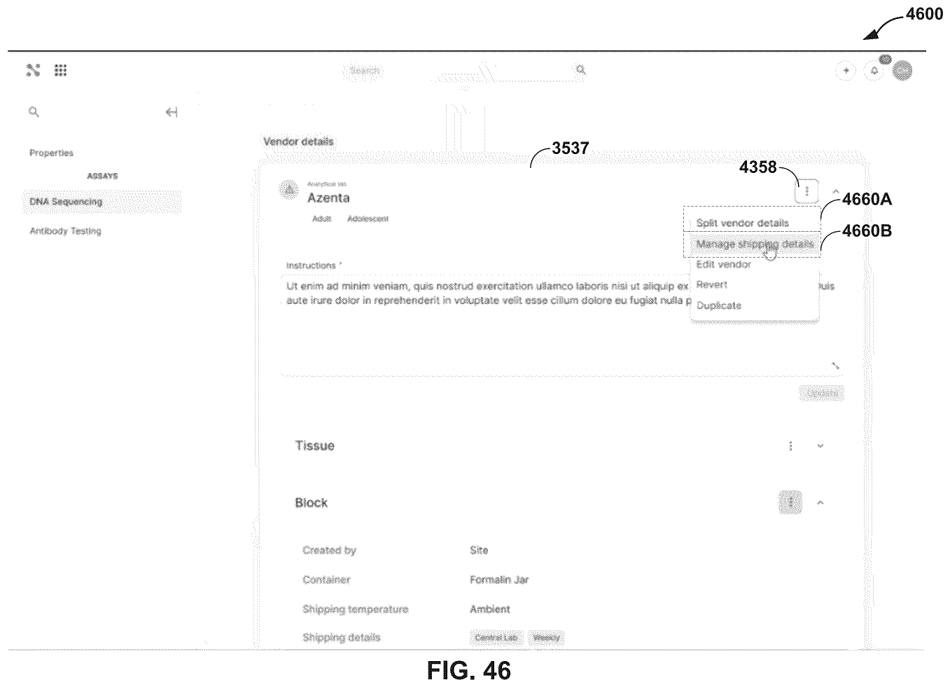

As a result of this behavior spanning across individual matrix types as well as the assays, SMUI 131B may provide entry into shipping details dialogue 4546 from any place on the vendor details pane 3537 (shown in the examples of FIGS. 35, 36, 43, and 44). Referring briefly back to the example of FIG. 43, vendor detail pane 3537 includes an addition action icon input 4358 that represents a button that, when selected, may expose additional actions. Responsive to receiving an indication that additional action icon input 4358 was selected, SMUI 4300 may transition to an SMUI 4600 shown in the example of FIG. 46.

SMUI 4600 may represent yet another example of SMU 131B, and includes vendor details pane 3537, which may itself include additional action item 4358 having been selected to expose a dropdown box that provides additional action item 4660B directed to managing shipping details. Responsive to an indication that additional action item 4660B was selected, SMUI 4600B may present shipping details dialogue 4546 (FIG. 45) as an overlay over SMUI 4600 (rather than SMUI 4400). Otherwise, shipping details dialogue 4546 is substantially similar between SMUI 4400 and 4600. In this respect, system 100 by way of SMUI 131B may provide a more flexible way without having to use work arounds or other incomplete processes involving excel spreadsheets and manually prepared tree diagrams.

In addition, the dropdown box exposed by selecting additional action icon input 4358 may also include a split vendor details additional action item 4660A. By default, system 100 may generate specimen collection groups as compactly as possible. For example, one vendor details per assay or multiple vendor details per assay based on how the subject groups are configured. Split vendor details additional action item 4660A may enable additional flexibility to override the default compact collection groups for assays to account for various scenarios.

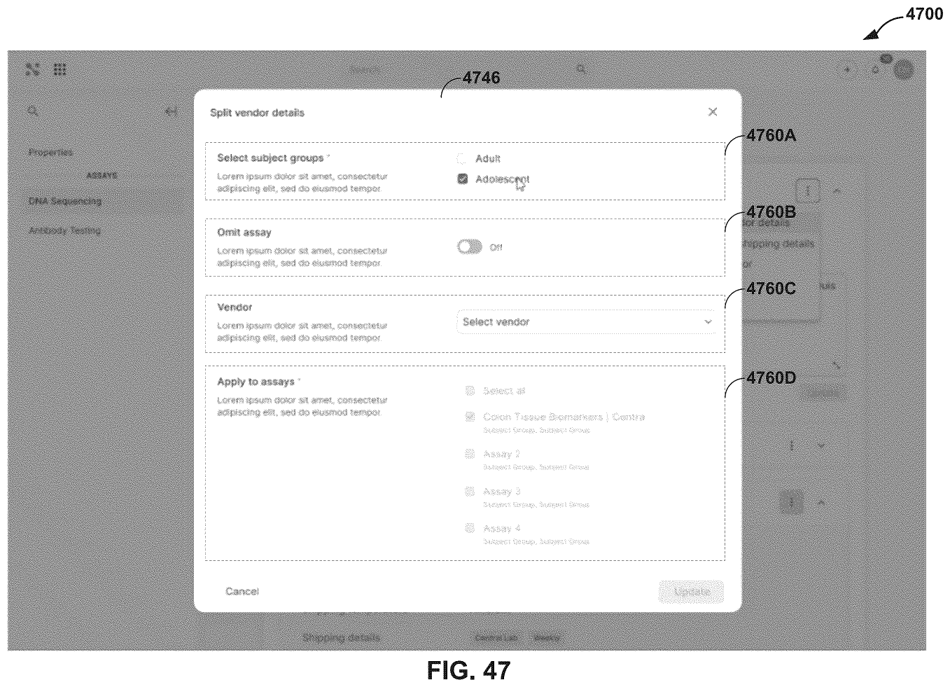

Responsive to receiving an indication that split vendor details additional action item 4660A was selected, SMUI 4600 may transition to an SMUI 4700 shown in the example of FIG. 47. SMUI 4700 may represent an example of SMUI 131B and includes split vendor details dialogue 4746 as an overlay over SMUI 4600. Split vendor details dialogue 4746 includes a select subject groups section 4760A, an omit assay section 4760B, a vendor selection section 4760C, and a select assay section 4760D.

Select subject groups section 4760A may represent a portion of split vendor details dialogue 4746 that enables selection of subject groups undergoing collection for the particular specimen by the particular vendor to be selected (e.g., “Adult” or “Adolescent”). In the example shown in FIG. 47, the “Adolescent” subject group has been selected, which will cause, if updated, any assays performed by the current vendor for adolescents to be split out to another vendor.

Omit assay 4760B may represent a portion of split vendor details dialogue 4746 that enables omission (via a toggle switch or other graphical user interface element) of the assays from being performed with respect to the selected subject group (e.g., in this case “Adolescents”). One example of why the SME may want to omit assays is there are body size or body weight restrictions and some assays may require too much of a given specimen for adolescents who do not satisfy the body size or body weight restrictions (e.g., too small for ethical restriction).

Another example might be that the SME wants to process this subject group at a different vendor. To accommodate this example, vendor selection section 4760C may represent a section of split vendor details dialogue 4746 that includes a dropdown box by which to select a different vendor. Until both one or more subject groups are selected and a different vendor is selected, select assay section 4760D is inactive (or in other words, not editable). Select assay section 4760D becomes active (or in other words, editable) upon the selection of one or more subject groups and the different vendor. Select assay section 4760D may represent a portion of split vendor details dialogue 4746 that enables selection of one or more assays for reassignment to the different vendor.

Assuming, as a first example, that “Adolescent” has been selected in select subject group section 4760A and that select vendor dropdown box input 4876 in vendor selection section 4760C was selected, SMUI 4700 transitions to SMUI 4800 (which again is an example of SMUI 131B) in which split vendor details dialogue 4746 activates select assays section 4760D and exposes a dropdown box 4878. The SME may then select a different vendor from among the items presents in exposed drop down box 4878 (e.g., “Local Lab,” “Central Lab,” etc.). The SME may also, after selecting the vendor via dropdown box 4878, select one or more compatible assays in assay selection section 4760D and then cancel or update per the cancel and update buttons described above in more detail.

There are a number of different examples of why different vendors may be selected. One example is due to certain restrictions by countries in which all testing for the subjects of the country must be conducted within the country. Reassignment of vendors may allow for such restrictions. In addition, by allowing more than one assay to be selected via assay selection section 4760D, a form of bulk editing may be enabled in which a number of different assays impacted by the restriction may be addressed within a single split vendor details dialogue 4746.

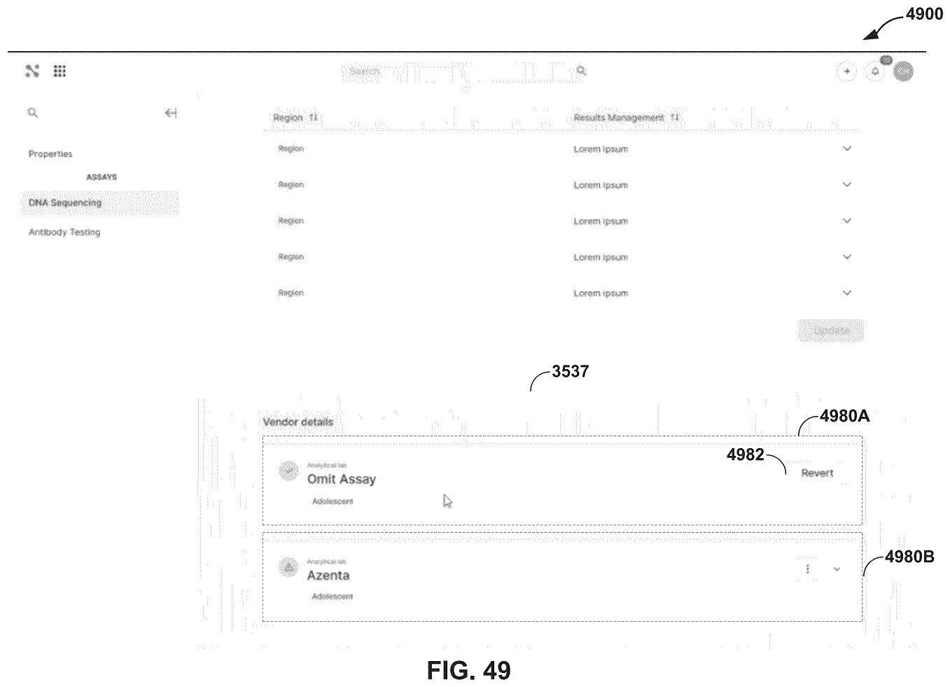

Assuming, as a second example, that “Adolescent” has been selected in select subject group section 4760A, omit assay is turned on in omit assay section 4760B, and the update button was selected to update the collection, SMUI 4800 may transition (either directly or indirectly through intermediate steps) to SMUI 4900 shown in the example of FIG. 49. SMUI 4900 may be similar to SMUIs 4300, 4400, and 4500 in that SMUI 4900 may represent another example of SMUI 131B, and includes vendor details pane 3537. However, in the example of FIG. 49, vendors details pane 3537 shows in vendor details element 4980 indicating that an assay is to be omitted for adolescents. Vendor details element 4980 may also include a revert button 4982, which may represent a button that, when selected, causes SMUI 4900 to undo or revert the split vendor details action during which the adolescent subject group was omitted. This reversion may result in the adolescents being added back into the vendor details as a subject group for which collection is to be performed.

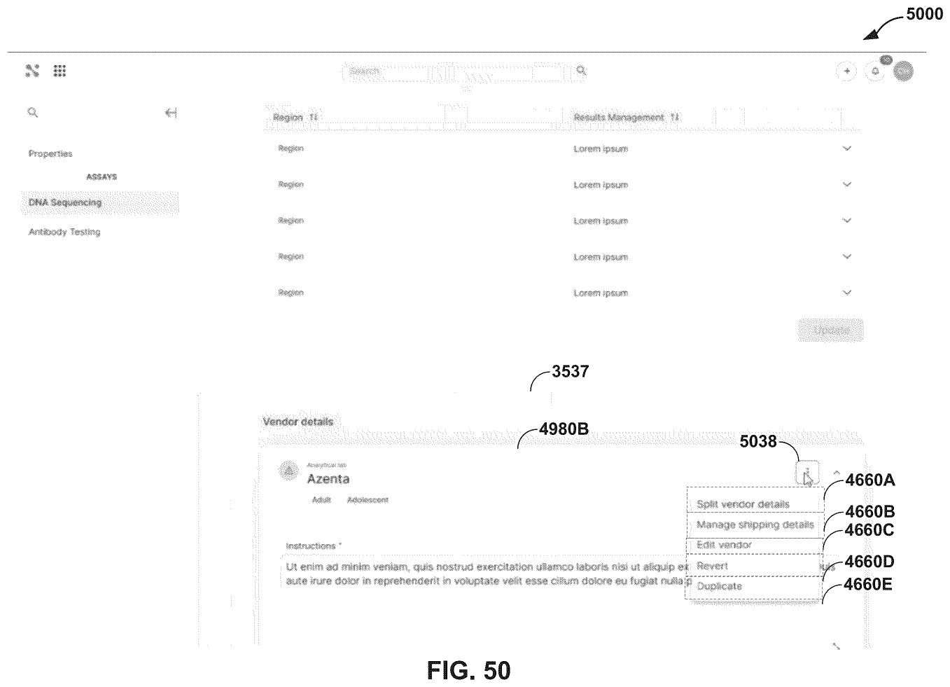

Responsive to receiving an indication that revert button 4982A was selected followed by expansion of a vendor details element 4980B (also included in vendor details pane 3537), SMUI 4900 may transition to SMUI 5000 shown in the example of FIG. 50. SMUI 5000 may represent an additional example of SMUI 131B and may include vendor detail pane 3537. Vendor details pane 3537 may include vendor details element 4890B in an expanded state, which exposes an additional action icon input 5038 (that is similar or the same as additional action icon input 4358). In the example of FIG. 50, an SME has selected additional action icon input 5038 to expose a dropdown box with addition actions items 4660A-4660E.

Additional action items 4660A and 4660B have been described above. Additional action item 4660 is directed to split vendor details, while additional action item 4660B is directed to manage shipping details. Additional action item 4660D is directed to revert and operates in a similar manner to revert button 4982. Additional action item 4660E may represent a button that, when pressed, may cause SMUI 5000 to duplicate the vendor details.

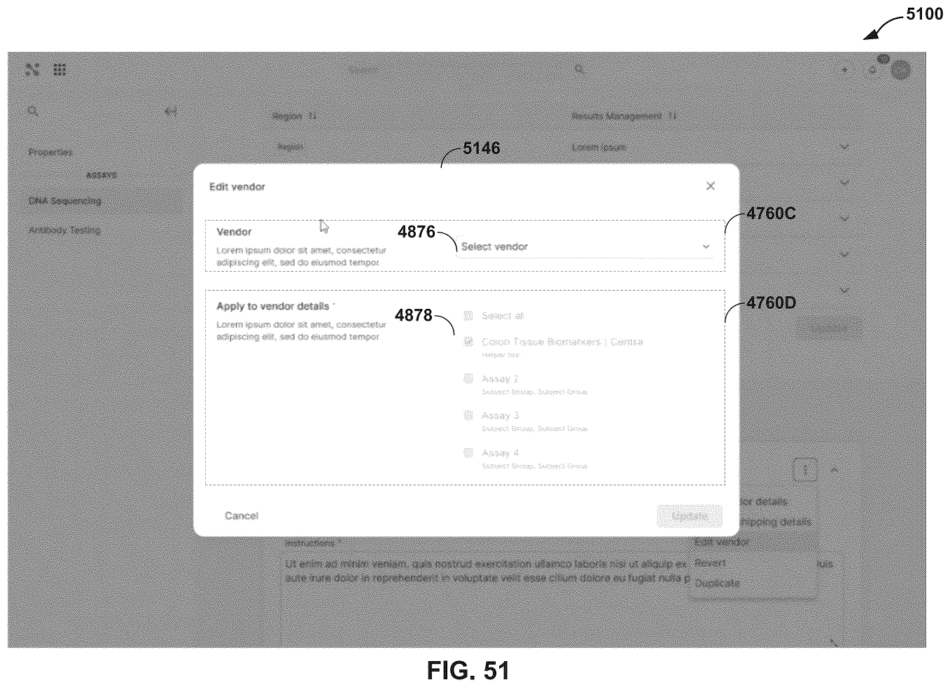

Duplication of vendor details may be useful in certain examples. To illustrate, an SME may want to ensure a particular important assay is successfully completed, and therefore wants to provide the assay twice to the same vendor (to overcome shipping delays, failures, etc.). In some instances, the SME may want to provide the specimen (or various forms thereof) to two different vendors. The SME may duplicate the current vendor via additional action item 4660E, and then edit the vendor details (to switch vendors) via additional action item 4660C. That is, additional action item 4660C may represent a button that, when selected, causes SMUI 5000 to overlay a vendor editor dialogue 5146 as shown in a SMUI 5100 of FIG. 51, thereby transitioning SMUI 5000 to SMUI 5100.

In the example of FIG. 51, SMUI 5100 may include a vendor editor dialogue 5146 overlayed on SMUI 5000. Vendor editor dialogue 5146 may include a vendor selection section 4760C in which select vendor dropdown box input 4876 resides, and a select assay section 4760D, all of which operate as described above with respect to SMUIs 4700 and 4800 shown the examples of FIGS. 37 and 38. Vendor editor dialogue 5146 may, in this way, provide a convenience to SME's to edit vendors to avoid having to edit each assay individually to reassign the vendor (which may represent a form of bulk editing).

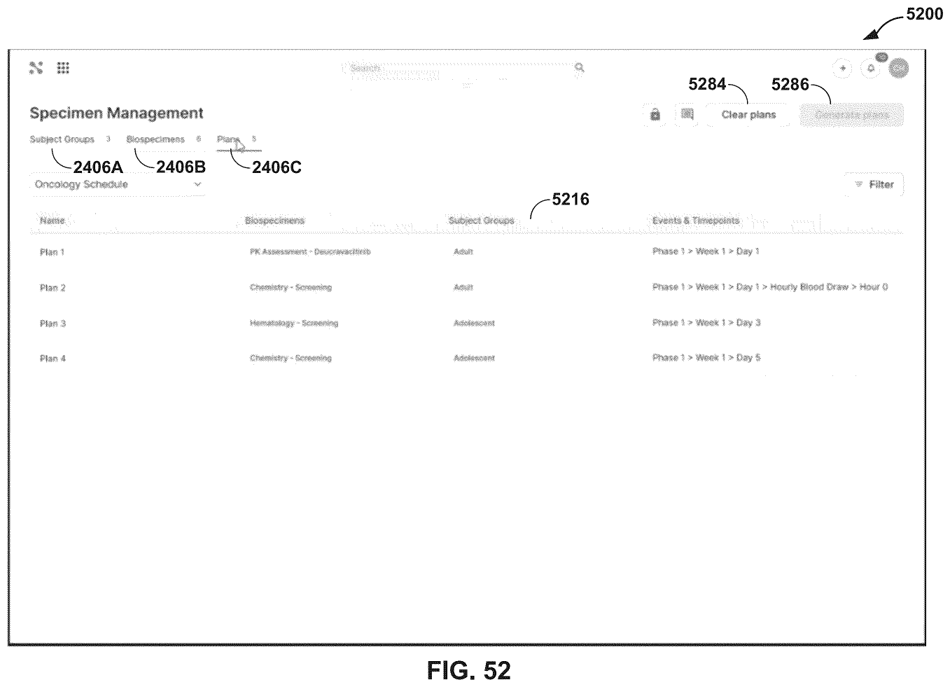

Assuming the SME navigates from SMUI 5100 back to home SMUI 2400 and selects plans tab 2406C, SMUI 5100 transitions from (including intermediate transitions) to an SMUI 5200 as shown in the example of FIG. 52. SMUI 5200 may represent an example of SMUI 131B and includes a plans table 5216 listing all of the plans in entry having fields defining a name, biospecimens, subject groups, and events and timepoints.

SMUI 5200 also includes a clear plans button 5284 and a generate plans button 5286. Clear plans button 5284 may represent a button that, when selected, causes SMUI 5200 to delete all of the plans in plans table 5216. Generate plans button 5286 may represent a button that, when selected, causes SMUI 5200 to generate all of the plans in plans table 5216.

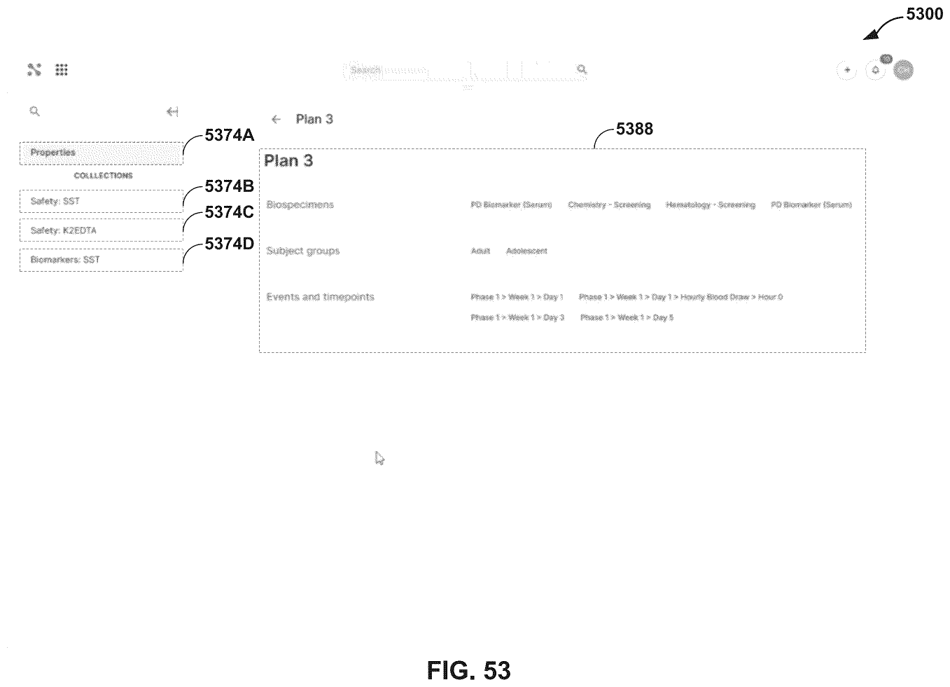

Assuming SMUI 5200 receiving an indication that one of the plan entries in plans table 5216 (e.g., plan 3) was selected and an indication that generate plans button 5285 was selected, SMUI 5200 may transition to SMUI 5300 shown in the example of FIG. 53. SMUI 5300 may represent an example of SMUI 131B and may, as shown in the example of FIG. 53, include horizontal tabs 5374A-5374D (“horizontal tabs 5374”), where horizontal tab 5374A is selected to show properties of plan 3 in a plan pane 5388. Plan plane 5388 (which is non-editable) identifies biospecimens (e.g., “PD Biomarker (Serum),” “Chemistry—Screening,” “Hematology—Screening,” and “PD Biomarker (Serum)”), subject groups (e.g., “Adult” and “Adolescent”), and events and timepoints (e.g., “Phase 1>Week>Day 1,” “Phase 1>Week>Day 1>Hourly Blood Draw>Hour,” “Phase 1>Week>Day 3,” and “Phase 1>Week>Day 5”).

Horizontal tab 5374B-5374D may be generally directed to collections and may provide sub-plans set forth for Plan 3. Horizontal tab 4374B may be directed to one or more “Safety:SST” collections and represents a button that, when selected, causes SMUI 5300 to jump to Safety:SST collections within SMUI 5300. Horizontal tab 4374C may be directed to one or more “Safety:K2EDTA” collections and represents a button that, when selected, causes SMUI 5300 to jump to Safety: K2EDTA collections within SMUI 5300. Horizontal tab 4374D may be directed to one or more “Biomarkers:SST” collections and represents a button that, when selected, causes SMUI 5300 to jump to Biomarkers:SST collections within SMUI 5300.

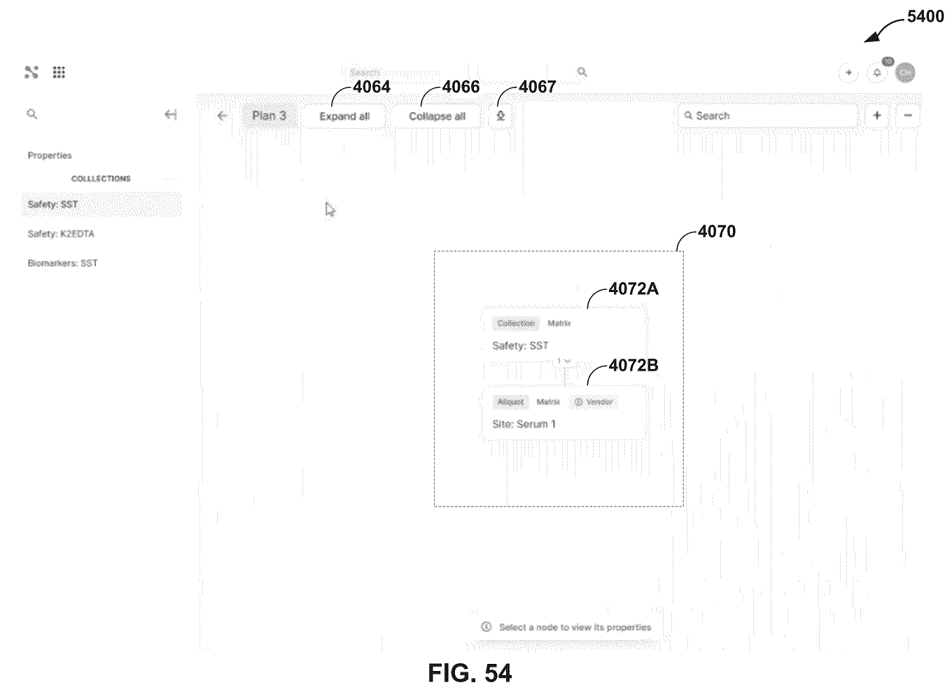

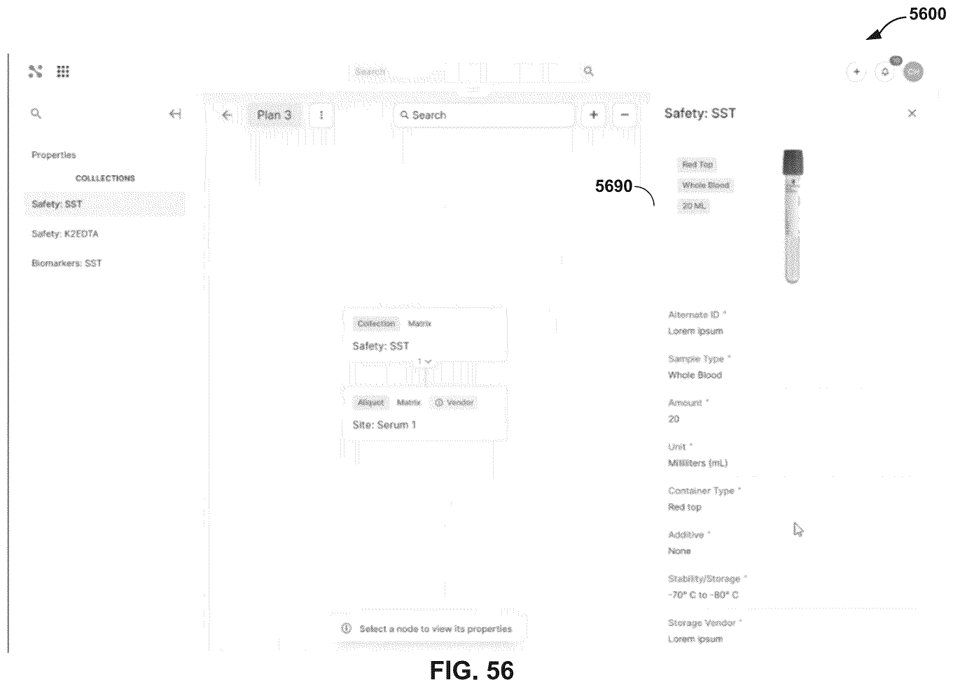

Assuming SMUI 5300 receiving an indication that horizontal tab 5374B was selected, SMUI 5300 may transition to an SMUI 5400 as shown in the example of FIG. 54, which may again represent an example of SMUI 131B. SMUI 5400 may include the proto-plan (if nothing was changed) shown by SMUI 4000 (FIG. 40). The proto-plan may include hierarchical tree view 4070 including nodes 4072, where SMUI 5400 may facilitate review of the to be generated plan via expand all button 4064, collapse all button 4066, and download button 4067. Previous inspection of the proto-plan may give the SME confidence that the plan as presented in FIG. 54 is in fact correctly specified for the study. The SME may nevertheless choose to further review the generated plan details as follows. FIG. 56 illustrates an example of such further inspection where the SME selects the collection node on 5600 and is presented with collection details on the right-side panel 5690. In a similar manner, aliquot node details may also be expanded for inspection.

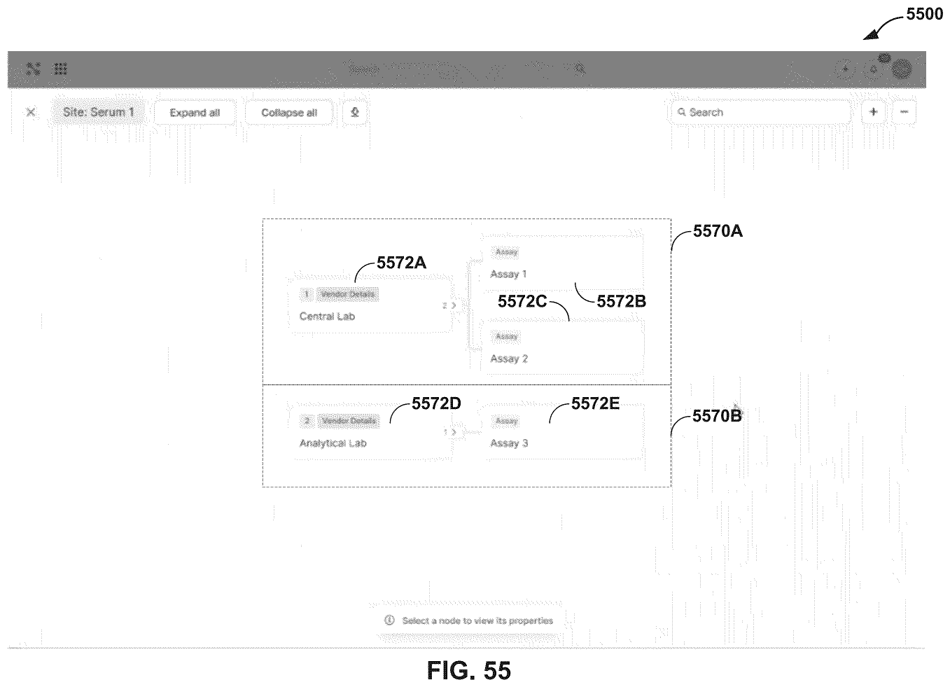

Assuming SMUI 5400 receives an indication that vendor in node 4072B was selected, SMUI 5400 may transition to an SMUI 5500 as shown in the example of FIG. 55, which once again is an example of SMUI 131B. SMUI 5500 may present two hierarchical tree views 5570A and 5570B, which denotes a specimen journey involving a serum that is shipped to the central lab and the central lab does two assays, and ships whatever portion of the serum is left to the analytical lab to do a third assay. Again, time flows from top-to-bottom, where top equals the start of the collection and the bottom equals the conclusion of the collection.

In any event, hierarchical tree view 5570A occurs first, where hierarchical tree view 5570A includes nodes 5572A-5572C. Node 5572A may represent a root node that identifies the central lab, while each of nodes 5572B and 5572C may represent the two assays performed by the central lab. Hierarchical tree view 5570B may occur second, where hierarchical tree view 5570B includes nodes 5572D and 5572E. Node 5572D may represent a root node that identifies the analytical lab and node 5572E may represent a leaf node that identifies the third assay.

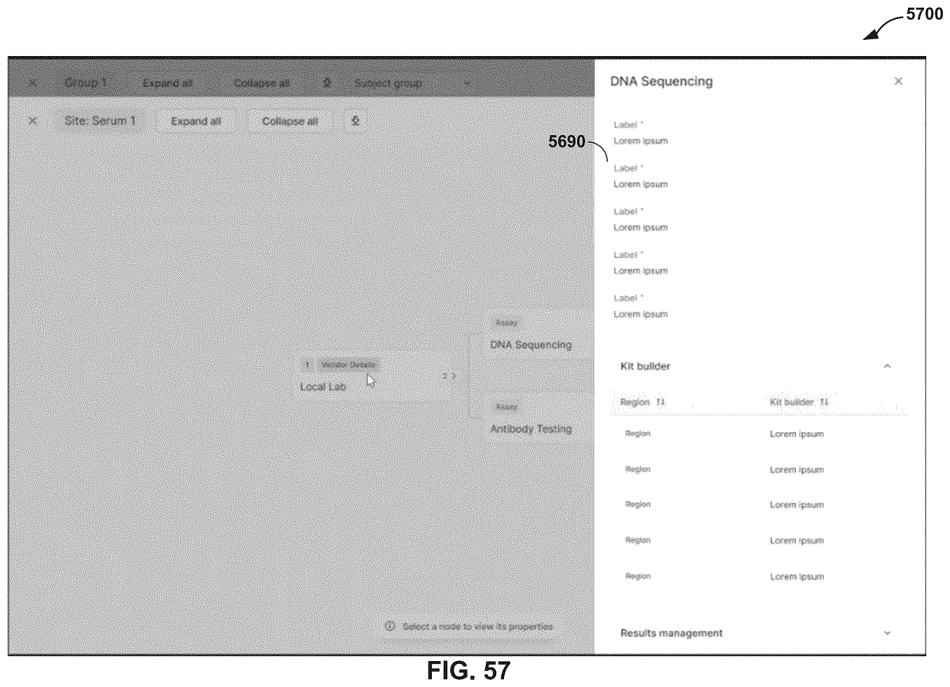

As a result of separating out the plan view from the vendor view, SMUI 5500 may allow for expanding information (or, in other words, a drill down) on a per node basis of the plan view similar to the aliquot depending on how the biospecimens were configured. Yet, each node is selectable to expose additional information. The cardinal numbers in the top left corners of each of nodes 5572A and 5572D denote an order in time, and as such time passes downwards. Similar to FIG. 56, FIG. 57 illustrates the ability to further review details of the assay node DNA Sequencing by invoking that node and as a result viewing right-hand panel 5690 presenting the plan details for that node. In a similar fashion, vendor nodes may also be expanded for inspection.

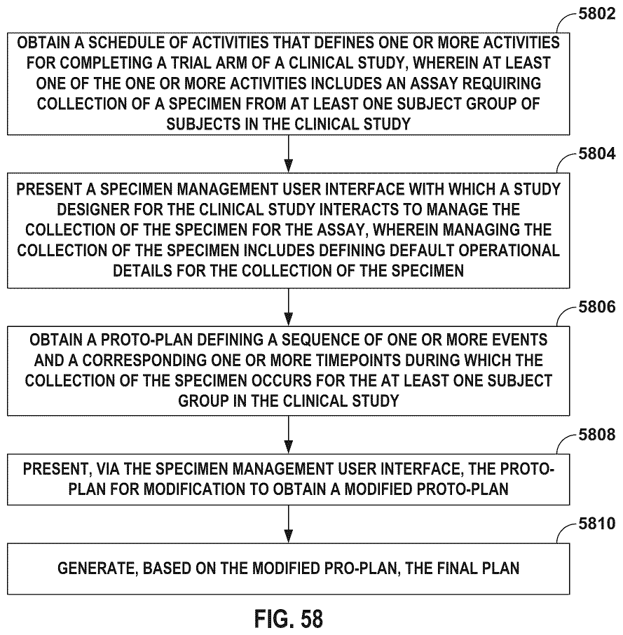

FIG. 58 is a flowchart illustrating example operation of the system of FIG. 1 in performing specimen management in accordance with various aspects of the techniques described in this disclosure. In operation, specimen management microservice 130B may obtain a schedule of activities that defines one or more activities specified within one or more events for completing a trial arm of a clinical study, where at least one of the one or more activities includes an assay (e.g., a lab test, a clinically administered monitoring assay, etc.) requiring collection of a specimen from at least one cohort of subjects in the clinical study (5802). Specimen management microservice 130B may interface with, as an example, the Study Design microservice 126 to obtain the Schedule of Activities, which may be unlocked (or in other words, not formally finalized) or locked (or in other words, formally finalized). Study Design microservice 126 and Specimen Management microservice 130B may operate according to an advanced message queuing protocol (AMQP) that defines an application layer protocol by which to instantiate a Biospecimen and its assays based on AMQP messages that are sent and thus arrive via a subscribe and pull mechanism.

Specimen management microservice 130B may interface with SMUI 131B to present a specimen management user interface with which a study designer (such as an SME) for the clinical study interacts to manage the collection of the specimen for the assay. The SME may interface with SMUI 131B to manage the collection of the specimen by, as one example, defining and/or overriding default operational details for the collection of the specimen (5804). The specimen management microservice may store default biospecimen, assay and operational details information for each biospecimen used by the customer. In total, this information makes up the specimen management standards for that customer. By their presence, specimen management specification is formalized and made consistent from study to study, thereby reducing the potential for error and improving efficiencies for the customer, ultimately resulting in specification of the specimen management collection plan more quickly and with higher quality than would otherwise be possible.

The default operational details may include one or more of a default isolate indication of whether to isolate a portion of the specimen specific to an assay, a default store remainder indication specifying whether any remainder of the specimen after assay processing is to be stored, a default cohort indication identifying the default cohort or cohorts, a default shipped from indication indicating a vendor from which the specimen is shipped, a default shipped matrix indication specifying a type of specimen, a default shipped medium indication specifying a medium for shipping, a default shipped to indication specifying where the specimen is to be shipped for purposes of assay processing, a default amount indication specifying an amount of the specimen required to process the assay, and a default unit indication specifying a unit for the amount of the specimen. As noted above, various fields for presenting the default operational details may be contingent on values entered into other fields for presenting the default operational details.

In any event, specimen management (SP) microservice 130B (which may be referred to as “SP microservice 130B”) may generate, responsive to priming the default operational details from standards maintained in the microservice, and receiving study-specific modifications to those standards via SMUI 131B, a proto-plan defining a sequence of one or more events and a corresponding one or more timepoints during which the collection of the specimen occurs for the cohort of the subjects in the clinical study (5806). Proto-plan generation is initiated via SME through SMUI and is possible even when the SOA is not locked and all biospecimens for the study are not completely specified (e.g., all required attributes of biospecimens, assays and operational details are not fully populated). The proto-plan may refer to a specimen collection plan for coordinating collection of the specimen during the events at the corresponding timepoints. SP microservice 130B may interface with SMUI 131B to present, via the SMUI 131B, the proto-plan for modification to obtain a modified proto-plan, potentially including defining override operational details that override the default operation details for the collection of the specimen (5808).

The override operational details include one or more of an override isolate indication that overrides whether to isolate the specimen for the assay at the one or more events for the one or more corresponding timepoints, an override store remainder indication specifying an override for whether any remainder of the specimen is to be stored, an override cohort indication identifying an override cohort, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect. The override operational details may only override the default operational details for the a selected collection within a particular plan, where the overriding information is used to generate a modified collection for the plan with changes to a particular collection element of assays (where collection elements represent a collection or merger of different assays for one or more specimens during events at corresponding timepoints), underlying aliquots (which refer to a subset of the assays within the particular collection element, where the subset of assays collect the same specimen for the same cohort), and the like. System 100 may next generate, based on the modified proto-plan, the final plan (5810).

As noted above, SP microservice 130B may obtain the Schedule of Activities prior to being locked from further editing (or in other words, finalized), generating, from the possible Study Activities that are current present in the unfinished Schedule of Activities, the set of biospecimens required to support those activities, thereby allowing SP microservice 130B to generate default information for the underlying assays and operational details that are required to support specimen collection. SP microservice 130B may only, in some examples, obtain Study Activities that are required, prepopulating selection of Study Activities as described above, but allowing the SME, via SMUI 131B, to select additional Study Activities that are optional. SP microservice 130B may interface with Study Design microservice 126 to obtain these Study Activities forming the non-formalized Study of Activities (via the subscribe and pull mechanism), and prepopulating at least a portion of the default operational details based on vendor requirements as recorded in biospecimen standards maintained by the microservice (e.g., central lab requirements that may vary depending on specimen processing capabilities, site specific requirements for collecting the specimen, etc.). The SME may modify these default operational details to change how specimen management occurs for the entire Schedule of Activities (corresponding to the Trail Arm) for the study under development.

In this way, various aspects of the techniques may enable the following examples.

Example 1. A method comprising: obtaining, by processing circuitry, a schedule of activities that defines one or more activities for completing a trial arm of a clinical study, wherein at least one of the one or more activities includes an assay requiring collection of a specimen from at least one subject group of subjects in the clinical study; presenting, by the processing circuitry, a specimen management user interface with which a study designer for the clinical study interacts to manage the collection of the specimen for the assay, wherein managing the collection of the specimen includes defining default operational details for the collection of the specimen; obtaining, by the processing circuitry, a proto-plan defining a sequence of one or more events and a corresponding one or more timepoints during which the collection of the specimen occurs for the at least one subject group in the clinical study; and presenting, by the processing circuitry and via the specimen management user interface, the proto-plan for modification, including defining override operational details that override the default operation details for the collection of the specimen.

Example 2. The method of example 1, wherein defining the default operational details comprises defining the default operational details prior to the schedule of activities being locked from further editing.

Example 3. The method of example 2, further comprising obtaining, responsive to the schedule of activities being locked from further editing, receiving the default operational details via the specimen management user interface, and committing the proto-plan, a plan.

Example 4. The method of any of examples 1-3, further comprising: obtaining a region that identifies a geographical region or a political region; obtaining a cohort that identifies a type of the at least one subject in terms of one or more of age, race, and weight; and obtaining the subject group based on the region and the cohort.

Example 5. The method of any of examples 1-4, wherein the at least one of the one or more activities includes a plurality of assays requiring collection of one or more specimens from two different subject groups in the clinical study, wherein obtaining the proto-plan comprises: identifying two or more assays requiring collection of an identical specimen of the one or more specimens; combining the two or more assays as an aliquot that compacts the collection for the identical specimen for a same subject group of the two or more subject groups; obtaining the proto-plan as the one or more events and the corresponding one or more timepoints during which the collection of the identical specimen for the same subject group is combined for the aliquot, and wherein presenting the proto-plan further comprises presenting the proto-plan as a hierarchical view of aliquots within a collection element along with the sequence of the one or more events and the corresponding one or more timepoints during which the collection of the identical specimen occurs for the same subject group of the two or more subject groups, the hierarchical view of aliquots including the aliquot.

Example 6. The method of example 5, wherein obtaining the proto-plan further comprises: identifying at least one different assay of the plurality of assays that requires collection of one or more of a different specimen of the one or more specimens for the same subject group of the two or more subject groups, the same specimen for a different subject group of the two or more subject groups, and a different specimen of the one or more specimens for the different subject group of the two or more subject groups; and obtaining a different aliquot for the collection element that includes the at least one different assay, and wherein the hierarchical view of aliquots within the collection element includes the aliquot and the different aliquot.

Example 7. The method of any of examples 5 and 6, further comprising: receiving, via the specimen management user interface, a split aliquot input from a user indicating that the aliquot is to be split; and splitting, responsive to receiving the split aliquot input, the aliquot into a first aliquot having a first aliquot of the two or more aliquots and a second aliquot of the two or more aliquots, the first aliquot different than the second aliquot, wherein presenting the proto-plan includes presenting the proto-plan as the hierarchical view of aliquots that includes the first aliquot and the second aliquot.

Example 8. The method of example 7, further comprising: receiving, via the specimen management user interface, a merge aliquot input from the user indicating that the first aliquot and the second aliquot is to be merged; and merging, responsive to receiving the merge aliquot input, the first aliquot and the second aliquot back into the aliquot.

Example 9. The method of any of examples 1-4, wherein the at least one of the one or more activities includes a plurality of assays, including the assay, requiring collection of one or more specimens from one or more subject groups of the subjects in the clinical study, wherein obtaining the proto-plan comprises identifying a different assay of the plurality of assays that requires collection of one or more of a different specimen of the one or more specimens for the same subject group of the two or more subject groups, the same specimen for a different subject group of the two or more subject groups, and a different specimen of the one or more specimens for the different subject group, and wherein the hierarchical view of aliquots within the collection element includes a first aliquot comprising the assay and a second aliquot comprising the different assay.

Example 10. The method of example 9, further comprising: receiving, via the specimen management user interface, a split collection input from a user indicating that the collection element is to be split; and splitting, responsive to receiving the split collection input, the collection element into a first collection element including a first view hierarchy comprising the first aliquot and a second collection element including a second hierarchical view of aliquots comprising the second aliquot, wherein presenting the proto-plan includes presenting the proto-plan as the first hierarchical view of aliquots within the first collection element and the second hierarchical view of aliquots within the second collection element.

Example 11. The method of example 10, receiving, via the specimen management user interface, a merge collection input from the user indicating that the first collection element and the second collection element is to be merged; and merging, responsive to receiving the merge collection input, the first collection element and the second collection element back into the collection element.

Example 12. The method of any of examples 1-11, wherein the default operational details include one or more of a default isolate indication of whether to isolate the specimen, a default store remainder indication specifying whether any remainder of the specimen is to be stored, a default subject group indication identifying the default subject group, a default shipped from indication indicating a vendor from which the specimen is shipped, a default shipped matrix indication specifying a type of specimen, a default shipped medium indication specifying a medium for shipping, a default shipped to indication specifying where any remainder is to be shipped, a default amount indication specifying an amount of the specimen to collect, and a default unit indication specifying a unit for the amount of the specimen to collect, and wherein the override operational details include one or more of an override isolate indication that overrides whether to isolate the specimen for the assay at the one or more events for the one or more corresponding timepoints, an override store remainder indication specifying an override for whether any remainder of the specimen is to be stored, an override subject group indication identifying an override subject group, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect.

Example 13. The method of any of examples 1-12, wherein the default operational details apply to each of the one or more events at the corresponding one or more timepoints, and wherein the override operational details apply to at least one of the one or more events at the corresponding at least one of the one or more timepoints.

Example 14. The method of any of examples 1-13, wherein the at least one subject group is not explicitly defined and includes all of the subjects for the clinical study.

Example 15. The method of any of examples 1-13, further comprising defining, via the specimen management user interface, the subject group as a categorized subset of the subjects in terms of a defined subject characteristic.

Example 16. The method of any of examples 1-15, further comprising: receiving, via the specimen management user interface, a decouple plan input specifying that at least one event of the one or more events and at least one corresponding timepoint of the one or more timepoints is to be decoupled into a first proto-plan that includes remaining events of the one or more events and not the at least one event and a second proto-plan distinct form the first proto-plan, the second proto-plan including the at least one event; and decoupling, responsive to receiving the decouple plan input, the proto-plan into the first proto-plan and the second proto-plan.

Example 17. The method of example 16, further comprising: receiving, via the specimen management user interface, a rejoin plan input specifying that the first proto-plan and the second proto-plan are to be rejoined; and rejoining, responsive to receiving the rejoin plan input the first proto-plan and the second proto-plan to obtain the proto-plan.

Example 18. A computing device comprising: a memory configured to store a schedule of activities that defines one or more activities for completing a trial arm of a clinical study, wherein at least one of the one or more activities includes an assay requiring collection of a specimen from at least one subject group of subjects in the clinical study; and processing circuitry configured to: present a specimen management user interface with which a study designer for the clinical study interacts to manage the collection of the specimen for the assay, wherein managing the collection of the specimen includes defining default operational details for the collection of the specimen; obtain a proto-plan defining a sequence of one or more events and a corresponding one or more timepoints during which the collection of the specimen occurs for the subject group of the subjects in the clinical study; and present, by the processing circuitry and via the specimen management user interface, the proto-plan for modification, including defining override operational details that override the default operation details for the collection of the specimen.

Example 19. The computing device of example 18, wherein the processing circuitry is configured to, when defining the default operational details, define the default operational details prior to the schedule of activities being locked from further editing.

Example 20. The computing device of example 18, wherein the processing circuitry is further configured to obtain, responsive to the schedule of activities being locked from further editing, receiving the default operational details via the specimen management user interface, and committing the proto-plan, a plan.

Example 21. The computing device of any of examples 18-20, wherein the processing circuitry is further configured to: obtain a region that identifies a geographical region or a political region; obtain a cohort that identifies a type of the at least one subject in terms of one or more of age, race, and weight; and obtain the subject group based on the region and the cohort.

Example 22. The computing device of any of examples 18-21, wherein the at least one of the one or more activities includes a plurality of assays requiring collection of one or more specimens from two different subject groups of the subject in the clinical study, wherein the processing circuitry is configured to, when obtaining the proto-plan: identify two or more assays requiring collection of an identical specimen of the one or more specimens; combine the two or more assays as an aliquot that compacts the collection for the identical specimen for a same subject group of the two or more subject group; obtain the proto-plan as the one or more events and the corresponding one or more timepoints during which the collection of the identical specimen for the same subject group is combined for the aliquot, and wherein the processing circuitry is configured to, when presenting the proto-plan present the plan as a hierarchical view of aliquots within a collection element along with the sequence of the one or more events and the corresponding one or more timepoints during which the collection of the identical specimen occurs for the same subject group, the hierarchical view of aliquots including the aliquot.

Example 23. The computing device of example 22, wherein the processing circuitry is configured to, when obtaining the proto-plan: identify at least one different assay of the plurality of assays that requires collection of one or more of a different specimen of the one or more specimens for the same subject group, the same specimen for a different subject group of the two or more subject groups, and a different specimen of the one or more specimens for the different subject group; and obtain a different aliquot for the collection element that includes the at least one different assay, and wherein the hierarchical view of aliquots within the collection element includes the aliquot and the different aliquot.

Example 24. The computing device of any combination of examples 22 and 23, wherein the processing circuitry is further configured to: receive, via the specimen management user interface, a split aliquot input from a user indicating that the aliquot is to be split; and split, responsive to receiving the split aliquot input, the aliquot into a first aliquot having a first aliquot of the two or more aliquots and a second aliquot of the two or more aliquots, the first aliquot different than the second aliquot, wherein the processing circuitry is configured to, when presenting the proto-plan, present the proto-plan as the hierarchical view of aliquots that includes the first aliquot and the second aliquot.

Example 25. The computing device of example 24, wherein the processing circuitry is configured to: receive, via the specimen management user interface, a merge aliquot input from the user indicating that the first aliquot and the second aliquot is to be merged; and merge, responsive to receiving the merge aliquot input, the first aliquot and the second aliquot back into the aliquot.

Example 26. The computing device of any of examples 18-21, wherein the at least one of the one or more activities includes a plurality of assays, including the assay, requiring collection of one or more specimens from one or more subject groups of the subjects in the clinical study, wherein the processing circuitry is configured to, when obtaining the proto-plan, identify a different assay of the plurality of assays that requires collection of one or more of a different specimen of the one or more specimens for the same subject group, the same specimen for a different subject group of the two or more subject groups, and a different specimen of the one or more specimens for the different subject group, and wherein the hierarchical view of aliquots within the collection element includes a first aliquot comprising the assay and a second aliquot comprising the different assay.

Example 27. The computing device of example 26, wherein the processing circuitry is further configured to: receive, via the specimen management user interface, a split collection input from a user indicating that the collection element is to be split; and split, responsive to receiving the split collection input, the collection element into a first collection element including a first view hierarchy comprising the first aliquot and a second collection element including a second hierarchical view of aliquots comprising the second aliquot, wherein the processing circuitry is configured to, when presenting the proto-plan, present the proto-plan as the first hierarchical view of aliquots within the first collection element and the second hierarchical view of aliquots within the second collection element.

Example 28. The computing device of example 27, wherein the processing circuitry is further configured to: receive, via the specimen management user interface, a merge collection input from the user indicating that the first collection element and the second collection element is to be merged; and merge, responsive to receiving the merge collection input, the first collection element and the second collection element back into the collection element.

Example 29. The computing device of any of examples 18-28, wherein the default operational details include one or more of a default isolate indication of whether to isolate the specimen, a default store remainder indication specifying whether any remainder of the specimen is to be stored, a default subject group indication identifying the default subject group, a default shipped from indication indicating a vendor from which the specimen is shipped, a default shipped matrix indication specifying a type of specimen, a default shipped medium indication specifying a medium for shipping, a default shipped to indication specifying where any remainder is to be shipped, a default amount indication specifying an amount of the specimen to collect, and a default unit indication specifying a unit for the amount of the specimen to collect, and wherein the override operational details include one or more of an override isolate indication that overrides whether to isolate the specimen for the assay at the one or more events for the one or more corresponding timepoints, an override store remainder indication specifying an override for whether any remainder of the specimen is to be stored, an override subject group indication identifying an override subject group, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect.

Example 30. The computing device of any of examples 18-29, wherein the default operational details apply to each of the one or more events at the corresponding one or more timepoints, and wherein the override operational details apply to at least one of the one or more events at the corresponding at least one of the one or more timepoints.

Example 31. The computing device of any of examples 18-30, wherein the at least one subject group is not explicitly defined and includes all of the subjects for the clinical study.

Example 32. The computing device of any of examples 18-31, wherein the processing circuitry is further configured to define, via the specimen management user interface, a subject group as a categorized subset of the subjects in terms of a defined subject characteristic.

Example 33. The computing device of any of examples 18-32, wherein the processing circuitry is further configured to: receive, via the specimen management user interface, a decouple plan input specifying that at least one event of the one or more events and at least one corresponding timepoint of the one or more timepoints is to be decoupled into a first plan that includes remaining events of the one or more events and not the at least one event and a second plan distinct form the first plan, the second plan including the at least one event; and decouple, responsive to receiving the decouple plan input, the proto-plan into the first proto-plan and the second proto-plan.

Example 34. The computing device of example 33, wherein the processing circuitry is further configured to: receive, via the specimen management user interface, a rejoin plan input specifying that the first plan and the second plan are to be rejoined; and rejoin, responsive to receiving the rejoin plan input the first proto-plan and the second proto-plan to obtain the proto-plan.

Example 35. A non-transitory computer-readable storage medium having instructions stored thereon that, when executed, cause one or more processors to: obtain a schedule of activities that defines one or more activities for completing a trial arm of a clinical study, wherein at least one of the one or more activities includes an assay requiring collection of a specimen from at least one subject group of subjects in the clinical study; present a specimen management user interface with which a study designer for the clinical study interacts to manage the collection of the specimen for the assay, wherein managing the collection of the specimen includes defining default operational details for the collection of the specimen; obtain a proto-plan defining a sequence of one or more events and a corresponding one or more timepoints during which the collection of the specimen occurs for the subject group of the subjects in the clinical study; and present, by the processing circuitry and via the specimen management user interface, the proto-plan for modification, including defining override operational details that override the default operation details for the collection of the specimen.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A method comprising:
obtaining, by processing circuitry, a schedule of activities that defines one or more activities for completing a trial arm of a clinical study, wherein at least one of the one or more activities includes an assay requiring collection of a specimen from at least one subject group of subjects in the clinical study;
presenting, by the processing circuitry, a specimen management user interface with which a study designer for the clinical study interacts to manage the collection of the specimen for the assay, wherein managing the collection of the specimen includes defining default operational details for the collection of the specimen;
obtaining, by the processing circuitry, a proto-plan defining a sequence of one or more events and a corresponding one or more timepoints during which the collection of the specimen occurs for the at least one subject group in the clinical study; and
presenting, by the processing circuitry and via the specimen management user interface, the proto-plan for modification, including defining override operational details that override the default operation details for the collection of the specimen.

2. The method of claim 1, wherein defining the default operational details comprises defining the default operational details prior to the schedule of activities being locked from further editing.

3. The method of claim 2, further comprising obtaining, responsive to the schedule of activities being locked from further editing, receiving the default operational details via the specimen management user interface, and committing the proto-plan, a plan.

4. The method of claim 1, further comprising:
obtaining a region that identifies a geographical region or a political region;
obtaining a cohort that identifies a type of the at least one subject in terms of one or more of age, race, and weight; and
obtaining the subject group based on the region and the cohort.

5. The method of claim 1,
wherein the at least one of the one or more activities includes a plurality of assays requiring collection of one or more specimens from two different subject groups in the clinical study,
wherein obtaining the proto-plan comprises:
identifying two or more assays requiring collection of an identical specimen of the one or more specimens;
combining the two or more assays as an aliquot that compacts the collection for the identical specimen for a same subject group of the two or more subject groups;
obtaining the proto-plan as the one or more events and the corresponding one or more timepoints during which the collection of the identical specimen for the same subject group is combined for the aliquot, and
wherein presenting the proto-plan further comprises presenting the proto-plan as a hierarchical view of aliquots within a collection element along with the sequence of the one or more events and the corresponding one or more timepoints during which the collection of the identical specimen occurs for the same subject group of the two or more subject groups, the hierarchical view of aliquots including the aliquot.

6. The method of claim 5,
wherein obtaining the proto-plan further comprises:
identifying at least one different assay of the plurality of assays that requires collection of one or more of a different specimen of the one or more specimens for the same subject group of the two or more subject groups, the same specimen for a different subject group of the two or more subject groups, and a different specimen of the one or more specimens for the different subject group of the two or more subject groups; and
obtaining a different aliquot for the collection element that includes the at least one different assay, and
wherein the hierarchical view of aliquots within the collection element includes the aliquot and the different aliquot.

7. The method of claim 5, further comprising:
receiving, via the specimen management user interface, a split aliquot input from a user indicating that the aliquot is to be split; and
splitting, responsive to receiving the split aliquot input, the aliquot into a first aliquot having a first aliquot of the two or more aliquots and a second aliquot of the two or more aliquots, the first aliquot different than the second aliquot,
wherein presenting the proto-plan includes presenting the proto-plan as the hierarchical view of aliquots that includes the first aliquot and the second aliquot.

8. The method of claim 7, further comprising:
receiving, via the specimen management user interface, a merge aliquot input from the user indicating that the first aliquot and the second aliquot is to be merged; and
merging, responsive to receiving the merge aliquot input, the first aliquot and the second aliquot back into the aliquot.

9. The method of claim 1,
wherein the at least one of the one or more activities includes a plurality of assays, including the assay, requiring collection of one or more specimens from one or more subject groups of the subjects in the clinical study,
wherein obtaining the proto-plan comprises identifying a different assay of the plurality of assays that requires collection of one or more of a different specimen of the one or more specimens for the same subject group of the two or more subject groups, the same specimen for a different subject group of the two or more subject groups, and a different specimen of the one or more specimens for the different subject group, and
wherein the hierarchical view of aliquots within the collection element includes a first aliquot comprising the assay and a second aliquot comprising the different assay.

10. The method of claim 9, further comprising:
receiving, via the specimen management user interface, a split collection input from a user indicating that the collection element is to be split; and
splitting, responsive to receiving the split collection input, the collection element into a first collection element including a first view hierarchy comprising the first aliquot and a second collection element including a second hierarchical view of aliquots comprising the second aliquot,
wherein presenting the proto-plan includes presenting the proto-plan as the first hierarchical view of aliquots within the first collection element and the second hierarchical view of aliquots within the second collection element.

11. The method of claim 10,
receiving, via the specimen management user interface, a merge collection input from the user indicating that the first collection element and the second collection element is to be merged; and
merging, responsive to receiving the merge collection input, the first collection element and the second collection element back into the collection element.

12. The method of claim 1,
wherein the default operational details include one or more of a default isolate indication of whether to isolate the specimen, a default store remainder indication specifying whether any remainder of the specimen is to be stored, a default subject group indication identifying the default subject group, a default shipped from indication indicating a vendor from which the specimen is shipped, a default shipped matrix indication specifying a type of specimen, a default shipped medium indication specifying a medium for shipping, a default shipped to indication specifying where any remainder is to be shipped, a default amount indication specifying an amount of the specimen to collect, and a default unit indication specifying a unit for the amount of the specimen to collect, and
wherein the override operational details include one or more of an override isolate indication that overrides whether to isolate the specimen for the assay at the one or more events for the one or more corresponding timepoints, an override store remainder indication specifying an override for whether any remainder of the specimen is to be stored, an override subject group indication identifying an override subject group, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect.

13. The method of claim 1,
wherein the default operational details apply to each of the one or more events at the corresponding one or more timepoints, and
wherein the override operational details apply to at least one of the one or more events at the corresponding at least one of the one or more timepoints.

14. The method of claim 1, wherein the at least one subject group is not explicitly defined and includes all of the subjects for the clinical study.

15. The method of claim 1, further comprising defining, via the specimen management user interface, the subject group as a categorized subset of the subjects in terms of a defined subject characteristic.

16. The method of claim 1, further comprising:
receiving, via the specimen management user interface, a decouple plan input specifying that at least one event of the one or more events and at least one corresponding timepoint of the one or more timepoints is to be decoupled into a first proto-plan that includes remaining events of the one or more events and not the at least one event and a second proto-plan distinct form the first proto-plan, the second proto-plan including the at least one event; and
decoupling, responsive to receiving the decouple plan input, the proto-plan into the first proto-plan and the second proto-plan.

17. The method of claim 16, further comprising:
receiving, via the specimen management user interface, a rejoin plan input specifying that the first proto-plan and the second proto-plan are to be rejoined; and
rejoining, responsive to receiving the rejoin plan input the first proto-plan and the second proto-plan to obtain the proto-plan.

18. A computing device comprising:
a memory configured to store a schedule of activities that defines one or more activities for completing a trial arm of a clinical study, wherein at least one of the one or more activities includes an assay requiring collection of a specimen from at least one subject group of subjects in the clinical study; and
processing circuitry configured to:
present a specimen management user interface with which a study designer for the clinical study interacts to manage the collection of the specimen for the assay, wherein managing the collection of the specimen includes defining default operational details for the collection of the specimen;
obtain a proto-plan defining a sequence of one or more events and a corresponding one or more timepoints during which the collection of the specimen occurs for the subject group of the subjects in the clinical study; and
present, by the processing circuitry and via the specimen management user interface, the proto-plan for modification, including defining override operational details that override the default operation details for the collection of the specimen.

19. The computing device of claim 18, wherein the processing circuitry is configured to, when defining the default operational details, define the default operational details prior to the schedule of activities being locked from further editing.

20. The computing device of claim 18, wherein the processing circuitry is further configured to obtain, responsive to the schedule of activities being locked from further editing, receiving the default operational details via the specimen management user interface, and committing the proto-plan, a plan.

21. The computing device of claim 18, wherein the processing circuitry is further configured to:
obtain a region that identifies a geographical region or a political region;
obtain a cohort that identifies a type of the at least one subject in terms of one or more of age, race, and weight; and
obtain the subject group based on the region and the cohort.

22. The computing device of claim 18,
wherein the at least one of the one or more activities includes a plurality of assays requiring collection of one or more specimens from two different subject groups of the subject in the clinical study,
wherein the processing circuitry is configured to, when obtaining the proto-plan:
identify two or more assays requiring collection of an identical specimen of the one or more specimens;
combine the two or more assays as an aliquot that compacts the collection for the identical specimen for a same subject group of the two or more subject group;
obtain the proto-plan as the one or more events and the corresponding one or more timepoints during which the collection of the identical specimen for the same subject group is combined for the aliquot, and
wherein the processing circuitry is configured to, when presenting the proto-plan present the plan as a hierarchical view of aliquots within a collection element along with the sequence of the one or more events and the corresponding one or more timepoints during which the collection of the identical specimen occurs for the same subject group, the hierarchical view of aliquots including the aliquot.

23. The computing device of claim 22,
wherein the processing circuitry is configured to, when obtaining the proto-plan:
identify at least one different assay of the plurality of assays that requires collection of one or more of a different specimen of the one or more specimens for the same subject group, the same specimen for a different subject group of the two or more subject groups, and a different specimen of the one or more specimens for the different subject group; and
obtain a different aliquot for the collection element that includes the at least one different assay, and
wherein the hierarchical view of aliquots within the collection element includes the aliquot and the different aliquot.

24. The computing device of claim 22,
wherein the processing circuitry is further configured to:
receive, via the specimen management user interface, a split aliquot input from a user indicating that the aliquot is to be split; and
split, responsive to receiving the split aliquot input, the aliquot into a first aliquot having a first aliquot of the two or more aliquots and a second aliquot of the two or more aliquots, the first aliquot different than the second aliquot,
wherein the processing circuitry is configured to, when presenting the proto-plan, present the proto-plan as the hierarchical view of aliquots that includes the first aliquot and the second aliquot.

25. The computing device of claim 24, wherein the processing circuitry is configured to:
receive, via the specimen management user interface, a merge aliquot input from the user indicating that the first aliquot and the second aliquot is to be merged; and
merge, responsive to receiving the merge aliquot input, the first aliquot and the second aliquot back into the aliquot.

26. The computing device of claim 18,
wherein the at least one of the one or more activities includes a plurality of assays, including the assay, requiring collection of one or more specimens from one or more subject groups of the subjects in the clinical study,
wherein the processing circuitry is configured to, when obtaining the proto-plan, identify a different assay of the plurality of assays that requires collection of one or more of a different specimen of the one or more specimens for the same subject group, the same specimen for a different subject group of the two or more subject groups, and a different specimen of the one or more specimens for the different subject group, and
wherein the hierarchical view of aliquots within the collection element includes a first aliquot comprising the assay and a second aliquot comprising the different assay.

27. The computing device of claim 26, wherein the processing circuitry is further configured to:
receive, via the specimen management user interface, a split collection input from a user indicating that the collection element is to be split; and
split, responsive to receiving the split collection input, the collection element into a first collection element including a first view hierarchy comprising the first aliquot and a second collection element including a second hierarchical view of aliquots comprising the second aliquot,
wherein the processing circuitry is configured to, when presenting the proto-plan, present the proto-plan as the first hierarchical view of aliquots within the first collection element and the second hierarchical view of aliquots within the second collection element.

28. The computing device of claim 27, wherein the processing circuitry is further configured to:
receive, via the specimen management user interface, a merge collection input from the user indicating that the first collection element and the second collection element is to be merged; and
merge, responsive to receiving the merge collection input, the first collection element and the second collection element back into the collection element.

29. The computing device of claim 18,
wherein the default operational details include one or more of a default isolate indication of whether to isolate the specimen, a default store remainder indication specifying whether any remainder of the specimen is to be stored, a default subject group indication identifying the default subject group, a default shipped from indication indicating a vendor from which the specimen is shipped, a default shipped matrix indication specifying a type of specimen, a default shipped medium indication specifying a medium for shipping, a default shipped to indication specifying where any remainder is to be shipped, a default amount indication specifying an amount of the specimen to collect, and a default unit indication specifying a unit for the amount of the specimen to collect, and
wherein the override operational details include one or more of an override isolate indication that overrides whether to isolate the specimen for the assay at the one or more events for the one or more corresponding timepoints, an override store remainder indication specifying an override for whether any remainder of the specimen is to be stored, an override subject group indication identifying an override subject group, an override shipped from indication overriding the vendor from which the specimen is shipped, an override shipped matrix indication overriding the type of specimen, an override shipped medium indication overriding the medium for shipping, an override shipped to indication overriding where any remainder is to be shipped, an overriding amount indication overriding the amount of the specimen to collect, and an overriding unit indication overriding the unit for the amount of the specimen to collect.

30. The computing device of claim 18,
wherein the default operational details apply to each of the one or more events at the corresponding one or more timepoints, and
wherein the override operational details apply to at least one of the one or more events at the corresponding at least one of the one or more timepoints.

31. The computing device of claim 18, wherein the at least one subject group is not explicitly defined and includes all of the subjects for the clinical study.

32. The computing device of claim 18, wherein the processing circuitry is further configured to define, via the specimen management user interface, a subject group as a categorized subset of the subjects in terms of a defined subject characteristic.

33. The computing device of claim 18, wherein the processing circuitry is further configured to:
receive, via the specimen management user interface, a decouple plan input specifying that at least one event of the one or more events and at least one corresponding timepoint of the one or more timepoints is to be decoupled into a first plan that includes remaining events of the one or more events and not the at least one event and a second plan distinct form the first plan, the second plan including the at least one event; and
decouple, responsive to receiving the decouple plan input, the proto-plan into the first proto-plan and the second proto-plan.

34. The computing device of claim 33, wherein the processing circuitry is further configured to:
receive, via the specimen management user interface, a rejoin plan input specifying that the first plan and the second plan are to be rejoined; and
rejoin, responsive to receiving the rejoin plan input the first proto-plan and the second proto-plan to obtain the proto-plan.

35. A non-transitory computer-readable storage medium having instructions stored thereon that, when executed, cause one or more processors to:
obtain a schedule of activities that defines one or more activities for completing a trial arm of a clinical study, wherein at least one of the one or more activities includes an assay requiring collection of a specimen from at least one subject group of subjects in the clinical study;
present a specimen management user interface with which a study designer for the clinical study interacts to manage the collection of the specimen for the assay, wherein managing the collection of the specimen includes defining default operational details for the collection of the specimen;
obtain a proto-plan defining a sequence of one or more events and a corresponding one or more timepoints during which the collection of the specimen occurs for the subject group of the subjects in the clinical study; and
present, by the processing circuitry and via the specimen management user interface, the proto-plan for modification, including defining override operational details that override the default operation details for the collection of the specimen.
